SEQUENCE LISTING (6 sequences)

SEQ ID NO: 1 - DNA, 17 bp, synthetic construct
  source 1..17: mol_type = other DNA
agtgggagcgcgtgatg

SEQ ID NO: 2 - DNA, 17 bp, synthetic construct
  source 1..17: mol_type = other DNA
gttcatctgcaccaccg

SEQ ID NO: 3 - DNA, 17 bp, synthetic construct
  source 1..17: mol_type = other DNA
gcctttacgatgtcctt

SEQ ID NO: 4 - protein, 21 aa, synthetic construct
MALPVTALLLPLALLLHAARP

SEQ ID NO: 5 - protein, 10 aa, synthetic construct
EQKLISEEDL

SEQ ID NO: 6 - protein, 20 aa, synthetic construct
GGAGCACTGTCCTCCGAACG

(19) United States
(12) Patent Application Publication
(10) Pub. No.: US 2026/0297520 A1
(43) Pub. Date: Oct. 1, 2026

(54) ENRICHMENT AND EXPANSION OF CHIMERIC ANTIGEN RECEPTOR T CELLS USING ANTIGEN-PRESENTING BIOMATERIALS

(71) Applicant: THE REGENTS OF THE UNIVERSITY OF CALIFORNIA, Oakland, CA (US)

(72) Inventors: Xiao Huang, San Francisco, CA (US); Tejal Ashwin Desai, San Francisco, CA (US); Qizhi Tang, San Francisco, CA (US); Wendell A. Lim, San Francisco, CA (US)

(21) Appl. No.: 19/483,540

(22) PCT Filed: May 10, 2024
(86) PCT No.: PCT/US2024/028957

§ 371 Date: Nov. 11, 2025

Related U.S. Application Data
(60) Provisional application No. 63/465,805, filed on May 11, 2023.

(51) Int. Cl. C12N 5/0783; B82Y 5/00; C07K 14/705; C07K 16/28; C12N 5/00

(52) U.S. Cl. CPC C12N 5/0636; C07K 14/7051; C07K 14/70517; C07K 14/70521; C07K 16/2818; C07K 16/2827; C07K 16/2863; C07K 16/2878; C12N 5/0018; B82Y 5/00; C12N 2501/056; C12N 2501/2302; C12N 2501/2307; C12N 2501/2315

(57) ABSTRACT

The present disclosure provides synthetic CAR-antigen presenting particles and films for stimulation of CAR-T cells. In particular, methods of using synthetic CAR-antigen presenting particles and films for activating effector function and stimulating proliferation of CAR-T cells are provided. Methods for expansion or enrichment of CAR-T cells using such synthetic CAR-antigen presenting particles and films are also provided. The disclosed methods can enrich and expand memory progenitor CAR-T cells massively without damaging their effector function. Indeed, cells with up to 6 rounds of stimulation and 10⁸-fold expansion show improved effector function and mitochondrial fitness.

### CROSS-REFERENCE TO RELATED APPLICATION

This application claims benefit under 35 U.S.C. § 119 (e) of provisional application 63/465,805, filed May 11, 2023, which application is hereby incorporated by reference in its entirety.

### STATEMENT REGARDING FEDERALLY SPONSORED RESEARCH OR DEVELOPMENT

This invention was made with government support under Grant No. U54 CA244438 awarded by the National Institutes of Health. The government has certain rights in the invention.

### INCORPORATION BY REFERENCE OF A SEQUENCE LISTING

A Sequence Listing is provided herewith as a Sequence Listing XML file, “UCSF-724WO” created on May 7, 2024 and having a size of 6,326 bytes. The contents of the Sequence Listing XML file are incorporated by reference herein in their entirety.

### INTRODUCTION

The use of chimeric antigen receptor (CAR) T cells targeting the B cell antigen CD19 has yielded remarkable clinical response in acute lymphocytic leukemia and diffuse large B cell lymphoma. However, broader clinical application of CAR-T cells still faces many challenges including i) the expansion of sufficient quantities of engineered T cells for clinical treatment without causing T cell anergy and exhaustion, and ii) the persistence and activity of potent memory CAR-T cells for durable leukemia eradication in vivo, which are important prognostic factors in achieving a meaningful clinical response in patients. In B cell cancers, CD19 antigens become depleted with the removal of both healthy and malignant B cells over the course of treatment. As a result, CAR-T counts drop and eventually become difficult to detect in patients. An approach to improve the in vivo persistence of CAR-T cells by providing replenished CD19 antigen on engineered T cells as artificial antigen presenting cells (T APC-CD19) is currently in clinical trial (clinicaltrials.gov NCT03186118).

There remains a need to improve in vivo expansion, persistence, and killing potential of CAR-T cells to allow more wide-spread clinical use in immunotherapy.

### SUMMARY

The present disclosure provides synthetic CAR-antigen presenting particles and films for stimulation of CAR-T cells. In particular, methods of using synthetic CAR-antigen presenting particles and films for activating effector function and stimulating proliferation of CAR-T cells are provided. Methods for expansion or enrichment of CAR-T cells using such synthetic CAR-antigen presenting particles and films are also provided. The disclosed methods can enrich and expand memory progenitor CAR-T cells massively without damaging their effector function. Indeed, cells with up to 6 rounds of stimulation and 10⁸-fold expansion show improved effector function and mitochondrial fitness.

In one aspect, a method of culture expansion of a chimeric antigen receptor (CAR)-T cell is provided, the method comprising: (a) culturing the CAR-T cell under suitable conditions for growth of the CAR-T cell; (b) contacting the CAR-T cell with a CAR-antigen presenting particle to produce an antigen-stimulated CAR-T cell, wherein the CAR-antigen presenting particle comprises an antigen, with or without a costimulatory ligand (e.g., anti-CD28 antibody and/or other ligand that stimulates a costimulatory receptor on a T cell), and/or cytokine presented on a surface of a synthetic particle, wherein binding of the antigen to the CAR induces proliferation of the CAR-T cell; (c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and (d) repeating steps (b) and (c).

In certain embodiments, the synthetic particle is a polymeric particle, a magnetic bead, a gold particle, or a liposome. In some embodiments, the method further comprises isolating a CAR-antigen presenting particle comprising a magnetic bead by using a magnetic separation technique (e.g., after said culture expansion of the chimeric antigen receptor (CAR)-T cell).

In certain embodiments, the costimulatory ligand on the surface of the synthetic particle is an anti-CD28 antibody, CD80, CD86, an anti-41BB antibody, an anti-ICOS antibody, or an anti-OX40 antibody.

In certain embodiments, the ratio of the antigen to the costimulatory ligand on the surface of the synthetic particle is 1:20 to 20:1, including any ratio within this range such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1. In some embodiments, the ratio of the antigen to the costimulatory ligand on the surface of the synthetic particle is 9:1.

In some certain embodiments, the cytokine on the surface of the synthetic particle is IL-2, IL-7, or IL-15, or a combination thereof.

In certain embodiments, the ratio of the antigen to the cytokine on the surface of the synthetic particle is 1:20 to 20:1, including any ratio within this range such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1.

In certain embodiments, step (b) is performed with a ratio of the synthetic CAR-antigen presenting particle to the CAR-T cell in a range of 2:1 to 20:1, 3:1 to 15:1, 4:1 to 12:1, or 5:1 to 10:1, or any ratio within these ranges such as 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

In certain embodiments, the method further comprises enriching for memory progenitor CAR-T cells generated from the antigen-stimulated CAR-T cell.

In certain embodiments, the method further comprises administering a therapeutically effective amount of the antigen-stimulated CAR-T cell to a subject in need thereof after said culture expansion. In some embodiments, the CAR-T cell is administered locally into a tumor or intravenously. In some embodiments, the method further comprises administering a therapeutically effective amount of the CAR-antigen presenting particle in combination with the antigen-stimulated CAR-T cell to a subject in need thereof after said culture expansion. In some embodiments, the subject has an impaired T cell response to the antigen. The subject may be immunocompromised, for example, because of treatment with chemotherapeutic agents or immunosuppressive agents.

In another aspect, a method of culture expansion of a CAR-T cell is provided, the method comprising: (a) culturing the CAR-T cell under suitable conditions for growth of the CAR-T cell; (b) contacting the CAR-T cell with a synthetic CAR-antigen presenting particle to produce an antigen-stimulated CAR-T cell, wherein the synthetic CAR-antigen presenting particle comprises: i) a polymeric core; ii) a nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; and iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR induces proliferation of the CAR-T cell; (c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and (d) repeating steps (b) and (c).

In certain embodiments, the CAR-antigen presenting particle further comprises a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

In certain embodiments, the costimulatory ligand is an anti-CD28 antibody, CD80, CD86, an anti-41BB antibody, an anti-ICOS antibody, or an anti-OX40 antibody.

In certain embodiments, the ratio of the antigen to the costimulatory ligand on the surface of the CAR-antigen presenting particle is 1:20 to 20:1.

In certain embodiments, step (b) is performed with a ratio of the synthetic CAR-antigen presenting particle to the CAR-T cell in a range of 2:1 to 20:1, 3:1 to 15:1, 4:1 to 12:1, or 5:1 to 10:1, or any ratio within these ranges such as 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

In certain embodiments, the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle. In some embodiments, the cytokine is IL-2, IL-7, or IL-15. In some embodiments, the antigen and the cytokine are present at a ratio of 10:1 to 1:10.

In certain embodiments, the method further comprises contacting the CAR-T cell with a synthetic cytokine-presenting particle, wherein the synthetic cytokine-presenting particle comprises a cytokine presented on a surface of a synthetic particle. In some embodiments, the synthetic particle is a polymeric particle, a magnetic bead, a gold particle, or a liposome. In some embodiments, the cytokine is IL-2, IL-7, or IL-15.

In certain embodiments, step (b) is repeated about every 5-15 days.

In certain embodiments, step (d) is repeated at least 1 time, at least 2 times, at least 3 times, at least 4 times, at least 5 times, at least 6 times, at least 7 times, at least 8 times, at least 9 times, at least 10 times, or more.

In certain embodiments, the polymeric core comprises poly(D,L-lactide-co-glycolide) (PLGA) or poly(lactic acid) (PLA). In some embodiments, the polymeric core comprises poly(D,L-lactide-co-glycolide) (PLGA), poly(D,L-lactide) (PLA), polyglycolic acid (PGA), poly(e-caprolactone) (PCL), or polyethylene glycol (PEG). In some embodiments, the polymer of nucleic acid-polymer conjugate comprises a poly(D,L-lactide-co-glycolide) (PLGA)-polyethylene glycol (PEG) block polymer (PLGA-block-PEG) or a poly(D,L-lactide) (PLA)-polyethylene glycol (PEG) block polymer (PLA-block-PEG) or a poly(e-caprolactone) (PCL)-polyethylene glycol (PEG) block polymer (PCL-block-PEG).

In certain embodiments, the first single stranded nucleic acid comprises deoxyribonucleic acid (DNA) or ribonucleic acid (RNA) or peptide nucleic acid (PNA).

In certain embodiments, the DNA or RNA or PNA comprises 5-200 bases.

In certain embodiments, the second single stranded nucleic acid comprises deoxyribonucleic acid (DNA) or ribonucleic acid (RNA) or peptide nucleic acid (PNA), optionally wherein the DNA or RNA or PNA comprises 5-200 bases.

In certain embodiments, the first single stranded nucleic acid comprises at least 4 contiguous bases complementary to at least 4 contiguous bases in the second single stranded nucleic acid.

In certain embodiments, the particle is a nanoparticle having a diameter ranging from 50 nm-500 nm. In some embodiments, the particle is a microparticle having a diameter ranging from 0.5 μm-50 μm.

In certain embodiments, the CAR comprises a transmembrane domain linked to an extracellular antigen binding domain and an intracellular signaling domain, wherein the extracellular antigen-binding domain specifically binds to the antigen. In some embodiments, the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the antigen. In some embodiments, the intracellular signaling domain is a CD3-zeta intracellular signaling domain or a ZAP-70 intracellular signaling domain. In some embodiments, the intracellular signaling domain comprises an immunoreceptor tyrosine-based activation motif (ITAM). In some embodiments, the transmembrane domain is a CD8, CD28, Megf10, FcRγ, Bai1, MerTK, TIM4, Stabilin-1, Stabilin-2, RAGE, CD300f, integrin subunit av, integrin subunit B5, CD36, LRP1, SCARF1, C1Qa, Axl, CD45, or CD86 transmembrane domain.

In certain embodiments, the CAR further comprises a costimulatory domain. In some embodiments, the costimulatory domain is a 4-1BB, CD28, ICOS, OX-40, BTLA, CD27, CD30, GITR, or HVEM costimulatory domain.

In certain embodiments, the antigen is a cancer antigen or a fibrosis antigen.

In certain embodiments, the antigen is selected from the group consisting of: CD19, HER2, epidermal growth factor receptor (EGFR), green fluorescent protein (GFP), fluorescein isothiocyanate (FITC), CD19, CD20, CD22, CD38, CD30, CA125, MUC-1, prostate-specific membrane antigen (PSMA), CD44 surface adhesion molecule, mesothelin, carcinoembryonic antigen (CEA), EGFRVIII, vascular endothelial growth factor receptor-2 (VEGFR2), high molecular weight-melanoma associated antigen (HMW-MAA), MAGE-A1, IL-13R-a2, GD2, MET, GPC3, CD70, EphA2, EpCAM, CLDN18, BCMA, CA9, and fibroblast activation protein (FAP).

In certain embodiments, the antigen is EGFR, the antigen-binding domain comprises an anti-EGFR nanobody, the transmembrane domain is a CD8 transmembrane domain or CD28 transmembrane domain, the intracellular signaling domain is a CD3-zeta intracellular signaling domain, and the costimulatory domain is a 4-1BB or CD28 costimulatory domain.

In certain embodiments, the method further comprises administering a therapeutically effective amount of the antigen-stimulated CAR-T cell to a subject in need thereof after said culture expansion. The CAR-T cell may be administered by any suitable mode of administration. In some embodiments, the CAR-T cell is administered locally into a tumor or fibrotic tissue, or administered intravenously. In some embodiments, the method further comprises administering a therapeutically effective amount of the CAR-antigen presenting particle in combination with the antigen-stimulated CAR-T cell to a subject in need thereof after the culture expansion.

In certain embodiments, the CAR-T cell is an effector T cell that has been genetically modified to express the CAR, or wherein the CAR-T cell is a regulatory T cell (Treg) that has been genetically modified to express the CAR. In some embodiments, the effector T cell is a helper CD4⁺ T cell, a cytotoxic CD8⁺ T cell, a natural killer T cell, or a gamma delta T cell. In some embodiments, the effector T cell or Treg is autologous or allogeneic.

In certain embodiments, the expression of the CAR in the CAR-T cell is inducible.

In certain embodiments, the CAR-T cell comprises a binding-triggered transcriptional switch that controls expression of the CAR by the CAR-T cell. In some embodiments, the binding-triggered transcriptional switch comprises a chimeric Notch polypeptide.

In certain embodiments, the CAR-T cell comprises an insertion of a recombinant polynucleotide comprising a coding sequence encoding the CAR at a TRAC locus of the CAR-T cell genome.

In certain embodiments, the antigen-stimulated CAR-T cell binds specifically to a cancer antigen. In some embodiments, the subject has a cancer expressing the cancer antigen and the antigen-stimulated CAR-T cell has anti-cancer activity. In some embodiments, the cancer is leukemia, lymphoma, myeloma, prostate cancer, breast cancer, lung cancer, kidney cancer, lung cancer, ovarian cancer, intestine cancer, or glioblastoma.

In certain embodiments, the antigen-stimulated CAR-T cell binds specifically to a fibrosis antigen. In some embodiments, the subject has fibrotic tissue expressing the fibrosis antigen and the antigen-stimulated CAR-T cell has anti-fibrosis activity. In some embodiments, the fibrosis antigen is FAP.

In certain embodiments, the antigen-stimulated CAR-T cell binds specifically to a B-cell antigen for autoreactive B cell removal.

In certain embodiments, the particle is superparamagnetic. In some embodiments, the method further comprises isolating the particle using magnetic separation techniques.

In another aspect, a method of activating a chimeric antigen receptor (CAR)-T cell is provided, the method comprising contacting the CAR-T cell with a synthetic CAR-antigen presenting particle to produce an activated CAR-T cell, wherein the synthetic CAR-antigen presenting particle comprises: i) a polymeric core; ii) a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR activates the CAR-T cell; and iv) a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the a costimulatory ligand on the surface of the polymeric particle.

In certain embodiments, the method is performed in vitro, ex vivo, or in vivo. In some embodiments, said contacting is performed in the presence of interleukin-2 (IL-2), a combination of interleukin-7 (IL-7) and interleukin-15 (IL-15), or a combination of IL-2, IL-7, and IL-15.

In certain embodiments, the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle. In some embodiments, the cytokine is IL-2, IL-7, or IL-15. In some embodiments, the antigen and the cytokine are present at a ratio of 10:1 to 1:10.

In certain embodiments, the method further comprises contacting the CAR-T cell with a synthetic cytokine-presenting particle, wherein the synthetic cytokine-presenting particle comprises a cytokine presented on a surface of a synthetic particle. In some embodiments, the synthetic particle is a polymeric particle, a magnetic bead, a gold particle, or a liposome. In some embodiments, the cytokine is IL-2, IL-7, or IL-15.

In another aspect, a method of culture expansion of a chimeric antigen receptor (CAR)-T cell is provided, the method comprising: (a) culturing the CAR-T cell under suitable conditions for growth of the CAR-T cell; (b) contacting the CAR-T cell with a synthetic CAR-antigen presenting film to produce an antigen-stimulated CAR-T cell, wherein the synthetic CAR-antigen presenting film comprises: i) a polymeric film comprising one or more pores; ii) a nucleic acid-polymer conjugate comprising a first single stranded nucleic acid covalently attached to a polymer, wherein the polymer is non-covalently associated with the polymeric film thereby presenting the first single stranded nucleic acid on a surface of the polymeric film; and iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the polymeric film, wherein the antigen specifically binds to the CAR expressed on the CAR-T cell, wherein binding of the antigen to the CAR induces proliferation of the CAR-T cell; (c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and (d) repeating steps (b) and (c). In some embodiments, the method is performed in vitro, ex vivo, or in vivo.

In certain embodiments, the polymeric film further comprises a second nucleic acid-polymer conjugate comprising a third single stranded nucleic acid covalently attached to the polymer, wherein the polymer is non-covalently associated with the polymeric film thereby presenting the third single stranded nucleic acid on the surface of the polymeric film and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric film.

In certain embodiments, the costimulatory ligand is an anti-CD28 antibody, CD80, CD86, an anti-41BB antibody, an anti-ICOS antibody, or an anti-OX40 antibody. In some embodiments, the ratio of the antigen to the costimulatory ligand on the surface of the polymeric film is 1:20 to 20:1.

In certain embodiments, the polymeric film further comprises a third nucleic acid-polymer conjugate comprising a fifth single stranded nucleic acid covalently attached to the polymer, wherein the polymer is non-covalently associated with the polymeric film thereby presenting the fifth single stranded nucleic acid on the surface of the polymeric film and a cytokine-nucleic acid conjugate comprising a sixth single stranded nucleic acid covalently attached to a cytokine, wherein the sixth single stranded nucleic acid is complementary to the fifth single stranded nucleic acid and is associated with the fifth single stranded nucleic acid via hybridization thereby presenting the cytokine on the surface of the polymeric film. In some embodiments, the cytokine is IL-2, IL-7, or IL-15. In some embodiments, the antigen and the cytokine are present at a ratio of 10:1 to 1:10.

In certain embodiments, the polymeric film comprises pores having a diameter of between 3 μm to 20 μm, optionally wherein the polymeric film comprises pores having a diameter of between 20 μm to 200 μm.

In certain embodiments, the polymeric film comprises a thickness of between 1 μm and 100 μm.

In certain embodiments, the polymeric film comprises poly(D,L-lactide-co-glycolide) (PLGA), poly(lactic acid) (PLA), polyglycolic acid (PGA), poly(e-caprolactone) (PCL), or polyethylene glycol (PEG).

In certain embodiments, the polymeric film comprises polycaprolactone (PCL) and polyethylene glycol (PEG).

In another aspect, a composition comprising a synthetic CAR-antigen presenting particle (CAPP) is provided, the CAPP comprising: (a) a polymeric core; (b) a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; (c) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to a CAR expressed on a CAR-T cell, wherein binding of the antigen to the CAR activates the CAR-T cell; and (d) a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

In certain embodiments, the costimulatory ligand is an anti-CD28 antibody, CD80, CD86, an anti-41BB antibody, an anti-ICOS antibody, or an anti-OX40 antibody. In some embodiments, the ratio of the antigen to the costimulatory ligand on the surface of the CAR-antigen presenting particle is 1:20 to 20:1.

In certain embodiments, the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

In certain embodiments, the cytokine is IL-2, IL-7, or IL-15. In some embodiments, the antigen and the cytokine are present at a ratio of 10:1 to 1:10.

In certain embodiments, the composition further comprises interleukin-2 (IL-2).

In certain embodiments, the composition further comprises interleukin-7 (IL-7) and/or interleukin-15 (IL-15).

In certain embodiments, the composition further comprises the CAR-T cell, wherein the antigen specifically binds to the CAR expressed on the CAR-T cell.

In certain embodiments, the composition further comprises a pharmaceutically acceptable excipient.

In another aspect, a composition comprising a synthetic CAR-antigen presenting particle (CAPP) for use in a method of immunotherapy is provided, the CAPP comprising: (a) a polymeric core; (b) a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; (c) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to a CAR expressed on a CAR-T cell, wherein binding of the antigen to the CAR activates the CAR-T cell; and (d) a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

In certain embodiments, the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

In another aspect, a kit comprising a composition comprising an antigen presenting particle or film, described herein, is provided. The kit may further comprise a CAR-T cell that specifically binds to the antigen presented on the CAR-antigen presenting particle or film. In addition, a kit may further comprise media suitable for culturing CAR-T cells. In some embodiments, the kit further comprises a vector for genetically modifying a T cell to produce a chimeric antigen receptor. In some embodiments, the kit further comprises means for administering a CAR-T cell, stimulated by an antigen presenting particle or film, described herein, to a subject in need of immunotherapy. In some embodiments, the kit further comprises instructions for using an antigen presenting particle or film in the subject methods, described herein.

### BRIEF DESCRIPTION OF THE DRAWINGS

The invention may be best understood from the following detailed description when read in conjunction with the accompanying drawings. Included in the drawings are the following figures:

FIG. 1 shows CAR-T cell expansion fold from a repeated stimulation (about every 10 days) by CAR-antigen presenting particles (CAPP) for 120 days. CAR-T cells are engineered from a healthy human donor with a CAR construct (a-EGFR nanobody, CD8-TM, CD28-costim., and CD3z-stim. domains) through a lentiviral vector. Cells are frozen, aliquoted, and thawed at day 12 for CAPP activation.

FIG. 2 shows flow cytometry based immune profiling of CAR-T cells from different rounds of CAPP-stimulations for surface marker expression.

FIGS. 3A-3B show human glioblastoma line U87 (FIG. 3A) and human prostate cancer line PC-3 (FIG. 3B) growth in vitro with cocultured CAR-T cells that are unstimulated (UnAct) or stimulated with CAPP for different rounds (3, 6, 9, and 10). E: T-effector (T cells) to target (cancer cells) ratio.

FIGS. 4A-4B show human glioblastoma line U87 growth with co-cultured CAR-T cells that has been stimulated with CAPP for (FIG. 4A) 8-rounds and (FIG. 4B) 9-rounds plus U87 cells for 0 (1^{st}-Killing), 1 (2^{nd}-Killing), 2 (3^{rd}-Killing), and 3 rounds (4th-Killing).

FIG. 5A-5B show (FIG. 5A) oxygen consumption rate (OCR) of CAR-T cells with a scheduled treatment of respiratory inhibitors profiling the mitochondrial respiration capacity, and (FIG. 5B) OCR linked to mitochondrial spare respiratory capacity of CAR-T cells from various rounds of CAPP treatments.

FIG. 6A shows expansion of CAR⋅T cells stimulated by CAPP for 10 to 12 rounds in the medium supplemented with IL-2 versus IL-7/IL-15. FIG. 6B shows OCR linked to mitochondrial spare respiratory capacity of CAR-T cells from various rounds of CAPP-treatments in medium supplemented with IL-2 versus IL-7/IL-15.

FIG. 7 shows expansion fold of CAR-T cells (a-EGFR nanobody, CD8-TM, CD28-costim., and CD3z-stim. domains) from three human donors with repeated stimulation of CAPP.

FIGS. 8A-8B show relative a-EGFR-CD28-costim. CAR-T cell count from the stimulation with CAPP of different sizes versus antigen-decorated magnetic particles versus HCC1569 cell line for 6 days in the medium supplemented with (FIG. 8A) IL⋅2 or (FIG. 8B) IL-7/IL-15.

FIG. 9 shows relative a-EGFR⋅41BBcostim. CAR⋅T cell count from the stimulation with CAPP of different sizes versus antigen-decorated magnetic particles versus HCC1569 cell line for 6 days in the medium supplemented with IL⋅2.

FIGS. 10A-10B show relative a-EGFR-41BBcostim. CAR-T cell count from the stimulation with CAPP coated with different ratios of antigens to costimulatory antibodies (a-CD28 antibody) versus HCC1569 cell line for 6 days in the medium supplemented with (FIG. 10A) IL⋅2 or (FIG. 10B) IL-7/IL-15.

FIG. 11A shows a fluorescent image of liposomes (−500 nm diameter) labeled with FITC dye. FIG. 11B shows a flow cytometry analysis of CellTrace Violet labeled CAR-T cells that are stimulated with GFP-decorated liposomes or GFP-decorated PLGA microparticles for 5 days.

FIG. 12 shows a cell expansion profile of 4-1BB costimulatory CAR-T cells (a-EGFR nanobody, CD8-TM, 4-1BB costim, and CD3z-stim. domains) from two human donors with repeated stimulation of U87 cells versus CAPP decorated with antigens and costimulatory antibodies (anti-CD28) at a 9:1 ratio.

FIG. 13 shows that PLGA microparticles decorated with antigen and a-CD28 antibody at a ratio of 9:1 yield the best EGFR-CAR 41BBζ T cell proliferation for 1-round of stimulation.

FIG. 14 shows that repeated stimulation of EGFR-CAR 41BBζ T cell using the best microparticle construct sustains T cell continuous proliferation for over 100 days. Data are from 3 healthy human donors.

FIGS. 15A-15B show that for CD28ζ CAR, the ratio of 9:1 does not show clear benefit to cell proliferation from the first round of microparticle-mediated stimulation. Results are shown for donors 1-3 with IL-7/IL-15 (FIG. 15A) or 4-1BBζ or CD28ζ (FIG. 15B).

FIG. 16 shows that CD28ζ CAR, PLGA microparticles decorated with antigen only can sustain continuous cell proliferation. However, for challenging donors like donor 3 and 4, the microparticles decorated with [9:1]-ratio sustain better for T cell proliferation.

FIG. 17 shows that for CD28ζ CAR, PLGA microparticles decorated with antigen only can sustain continuous cell proliferation. However, for challenging donors like donor 3 and 4, the microparticles decorated with [9:1]-ratio sustain better for T cell proliferation.

FIG. 18 shows that this biomaterial-mediated stimulation approach yielded ~10,000 folds of additional expansion beyond the cell expansion obtained from the typical CAR-T manufacturing procedure after 25-30 days' culture. This additional expansion goes up to 100 million folds after 50-60 days' culture.

FIG. 19 show that repeated stimulation using U87 cells (a glioblastoma cell line) in the CAR-specific stimulation manner leads to faster cell dysfunction.

FIG. 20 shows that the current standard way of ex vivo activation of T cells in the non-CAR specific manner using Dynabeads does not selectively expand CAR-T cells and does not generate as large folds of cell expansion.

FIG. 21 shows CAR expression on expanded cells after 0-10 rounds of PB-stimulations. This approach enriches CAR+ population, and it does show a preferrable expansion of T cells with high CAR copies.

FIG. 22 shows that expanded cells show a retained effector function. Indeed, 3 rounds of stimulation yielded cells with improved target toxicity.

FIGS. 23A-23B show that the same trend of retained effector function is observed when using CAR-T cells to kill a different target cell line U87 (FIG. 23A). The expanded cells show full capacity of serial killing (FIG. 23B).

FIGS. 24A-24B show that the expanded cells after 3-6 rounds of biomaterial stimulations showed augmented mitochondria fitness based on the Seahorse assay. Mitochondrial stress (FIG. 24A) and spare respiration capacity (FIG. 24B) were measured.

FIGS. 25A-25B show single-cell RNA sequencing analysis showing that there is a clear clonal enrichment along with the chronic stimulation (FIG. 25A) and cell expansion (FIG. 25B).

FIGS. 26A-26C shows the enriched clones from PLGA microparticle-mediated activation and expansion resemble important profiles of memory progenitors. FIG. 26A shows Leiden clustering of enriched clones. FIG. 26B shows differentially expressed genes associated with particle stimulation. FIG. 26C shows mRNA and protein markers associated with memory, effector function, and exhaustion for clusters.

FIG. 27 shows that microparticle stimulation uncouples cell proliferation from quicker cell exhaustion.

FIGS. 28A-28B shows that particle stimulation uncouples T cell proliferation from excessive immune activation (bulk RNA-seq, 24 hours). FIG. 28A shows up-regulated and down-regulated genes. FIG. 28B shows functional classification and pathway analysis of differentially expressed genes associated with particle stimulation.

FIGS. 29A-29C show that microparticle stimulation temporally induces higher telomerase activity, compensating for telomere shortening during cell expansion. FIG. 29A shows a plot of relative telomere to single copy gene (T/S) ratio versus expansion fold for different clones. FIG. 29B compares telomerase activity of unstimulated CAR-T cells from different donors to the telomerase activity of the CAR-T cells after restimulation with microparticles for 3 days. FIG. 29C shows plots of telomerase activity versus expansion fold for different clones.

FIG. 30 shows that biomaterials with customized ratio of antigen to costimulatory antibody also sustain BCMA-CAR CD287 for sustained proliferation. The CAR construct has the same ectodomain as the FDA-approved drug Abecma.

FIGS. 31A-31B show that increasing the CAPP to CAR-T cell ratio sustains 41BB costimulation of proliferation of CD19-specific CAR-T cells in vitro. Cell expansion-fold over time is compared at lower (FIG. 31A) and higher (FIG. 31B) CAPP/aCD28 to cell ratios.

### DETAILED DESCRIPTION

The present disclosure provides synthetic CAR-antigen presenting particles and films for stimulation of CAR-T cells. In particular, methods of using synthetic CAR-antigen presenting particles and films for activating effector function and stimulating proliferation of CAR-T cells are provided. Methods for expansion or enrichment of CAR-T cells using such synthetic CAR-antigen presenting particles and films are also provided. The disclosed methods can enrich and expand memory progenitor CAR-T cells massively without damaging their effector function. Indeed, cells with up to 6 rounds of stimulation and 10⁸-fold expansion show improved effector function and mitochondrial fitness.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a first nucleic acid” includes a plurality of such first nucleic acid and reference to “the nucleic acid” includes reference to one or more nucleic acids, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The term “about,” particularly in reference to a given quantity, is meant to encompass deviations of plus or minus five percent.

The term “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” refers to a polymer of a length greater than about 5 bases, greater than about 10 bases, greater than about 15 bases, or greater than about 20 bases. A nucleic acid may be 5-200 bases in length, e.g., 5-50 bases, 50-200 bases, 10-80 bases, 10-50 bases, 10-30 bases, 10-20 bases, or 12-20 bases in length. The term “nucleic acid” refers to a polymer of nucleotides, e.g., deoxyribonucleotides, ribonucleotides, or peptide nucleic acid (PNA) and may be produced enzymatically or synthetically (e.g., PNA as described in U.S. Pat. No. 5,948,902) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively). The nucleic acid can be single stranded or double stranded.

The term “hybridization” refers to the specific binding of a nucleic acid to a complementary nucleic acid via Watson-Crick base pairing.

The term “hybridization conditions” as used herein refers to conditions that allow hybridization of a nucleic acid to a complementary nucleic acid, e.g., a nucleic acid immobilized on a polymeric particle may specifically bind to a complementary nucleic acid via Watson-Crick base pairing under hybridization conditions.

“Isolated” refers to an entity of interest that is in an environment different from that in which it may naturally occur. “Isolated” is meant to include entities that are within samples that are substantially enriched for the entity of interest and/or in which the entity of interest is partially or substantially purified. “Pharmaceutically acceptable excipient or carrier” refers to an excipient that may optionally be included in the compositions of the invention and that causes no significant adverse toxicological effects to the patient.

“Substantially” or “essentially” means nearly totally or completely, for instance, 95% or greater of some given quantity.

“Substantially purified” generally refers to isolation of a substance (e.g., compound, small molecule, drug, polynucleotide, protein, polypeptide, antibody, aptamer, AGE inhibitor or crosslink breaker) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The terms “individual,” “subject,” and “patient” are used interchangeably herein to refer to an individual to be treated by (e.g., administered) the compositions and methods of the present invention. Subjects include, but are not limited to, mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows. In some cases, the methods of the invention find use in experimental animals, in veterinary application, and in the development of animal models for disease, including, but not limited to, rodents including mice, rats, and hamsters; primates, and transgenic animals. In the context of the disclosure, the term “subject” generally refers to an individual who will be administered or who has been administered one or more compositions described herein (e.g., CAR-T cells expanded and/or activated by synthetic CAR-antigen presenting particles, as described herein).

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma.

The term cancer may include, but is not limited to, prostate cancer, breast cancer, ovarian cancer, melanoma, pancreatic cancer, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, male breast cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, head and neck cancer, skin cancer, oropharyngeal cancer, bone cancers, including osteosarcoma and malignant fibrous histiocytoma of bone, paranasal sinus cancer, parathyroid cancer, penile cancer, pheochromocytoma, pituitary tumors, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

By “anti-tumor activity” or “anti-cancer activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

The terms “treat”, “treating”, treatment,” “prevent,” “preventing,” and the like refer to a course of action (such as administering an agent or a pharmaceutical composition comprising an agent) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, or condition afflicting a subject. Thus, treatment includes inhibiting (i.e., arresting the development or further development of the disease, disorder or condition or clinical symptoms association therewith) an active disease.

The term “in need of treatment” as used herein refers to a judgment made by a physician or other caregiver that a subject requires or will benefit from treatment. This judgment is made based on a variety of factors that are in the realm of the physician's or caregiver's expertise.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount will refer to an amount having anti-cancer activity, i.e., an amount of a therapeutic agent that decreases the rate of tumor growth (e.g., reduces and/or clears tumor burden in the patient (e.g., reduces the number of cancer cells in a patient)), decreases tumor mass, decreases the number of metastases, decreases tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like.

The terms “antibodies” and “immunoglobulin” include antibodies or immunoglobulins of any isotype, fragments of antibodies which retain specific binding to an antigen, including, but not limited to, Fab, Fv, scFv, and Fd fragments, monoclonal antibodies, hybrid antibodies, chimeric antibodies, humanized antibodies, single-chain antibodies, single-domain antibodies, nanobodies, bispecific antibodies, tri-specific antibodies, and other multi-specific antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Single-chain Fv” or “sFv” antibody fragments comprise the V_{H} and V_{L} domains of an antibody, wherein these domains are present in a single polypeptide chain. In some embodiments, the Fv polypeptide further comprises a polypeptide linker between the V_{H} and V_{L} domains, which enables the sFv to form the desired structure for antigen binding.

The terms “specific binding,” “specifically binds,” and the like, refer to non-covalent or covalent preferential binding to a molecule relative to other molecules or moieties in a solution or reaction. In some embodiments, the affinity of one molecule for another molecule to which it specifically binds is characterized by a K_{D} (dissociation constant) of 105 M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10-12 M or less). “Affinity” refers to the strength of binding, increased binding affinity being correlated with a lower K_{D}. In an embodiment, affinity is determined by surface plasmon resonance (SPR), e.g. as used by Biacore systems. The affinity of one molecule for another molecule is determined by measuring the binding kinetics of the interaction, e.g., at 25° C.

The term “antigen-binding fragment” as used herein refers to any antibody fragment that specifically binds to a target antigen including, but not limited to, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody including one or more complementarity determining regions (CDRs).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. VL and VH sequences can be reformatted as fragments, as single chain binding domains, linked to chimeric antigen receptors, and the like.

The term “antigen binding domain (ABD)” refers to a domain that specifically binds to a target antigen. The antigen binding domain region of an antibody may comprise a heavy-chain variable domain (VH) and a light-chain variable domain (VL) in non-covalent association as a single polypeptide or as a dimer. The three complementarity-determining regions of the heavy chain variable domain (CDR H1, H2, H3) and three complementarity-determining regions of the light chain variable domain (CDR L1, L2, L3) interact to define an antigen-binding site on the surface of an antibody. Collectively, the six CDRs of the light chain and heavy chain variable domains confer antigen-binding specificity to an antibody. An antigen binding domain region of a CAR may comprise all six CDRs of an antibody or a single variable domain or half of an Fv fragment comprising only three CDRs specific for an antigen, which still retains the ability to recognize and bind the target antigen. In some embodiments, the antigen-binding domain binds to one or more target antigens expressed on the surface of a target cell (e.g., cell surface markers).

Chimeric antigen receptor (CAR). A CAR may have any suitable architecture, as known in the art, comprising an antigen binding domain, usually provided in an scFv format, linked to T cell receptor effector functions. The term refers to artificial multi-module molecules capable of triggering or inhibiting the activation of an immune cell. A CAR will generally comprise an antigen binding domain, linker, transmembrane domain and cytoplasmic signaling domain. In some instances, a CAR will include one or more co-stimulatory domains and/or one or more co-inhibitory domains.

The antigen-binding domain of the CAR may include any naturally occurring, synthetic, semi-synthetic, or recombinantly produced binding partner for a target antigen of interest. In some embodiments, the binding region is an antigen-binding region, such as an antibody or functional binding domain or antigen-binding fragment thereof. The antigen-binding region of the CAR can include any domain that binds to the antigen and may include, but is not limited to, a monoclonal antibody, a polyclonal antibody, a synthetic antibody, a human antibody, a humanized antibody, a non-human antibody, a single-chain antibody, and any antigen-binding fragment thereof. Thus, in some embodiments, the antigen binding domain portion includes a mammalian antibody or an antigen-binding fragment thereof. An antigen-binding domain may comprise an antigen-binding fragment (Fab), a single-chain variable fragment (scFv), a nanobody, a VH domain, a VL domain, a single domain antibody (sdAb), a shark variable domain of a new antigen receptor (VNAR), a single variable domain on a heavy chain (VHH), a bispecific antibody, or a diabody; or a functional antigen-binding fragment thereof. In some embodiments, the antigen-binding domain is derived from the same cell type or the same species in which the CAR will ultimately be used. For example, for use in humans, the antigen-binding domain of the CAR may include a human antibody, a humanized antibody, or an antigen-binding fragment thereof.

In some embodiments, the antigen binding domain is derived from a single chain antibody that selectively binds to a target antigen. In some embodiments, the antigen binding domain is provided by a single chain variable fragment (scFv). A scFv is a recombinant molecule in which the variable regions of the light and heavy immunoglobulin chains are connected in a single fusion polypeptide. Generally, the VH and VL sequences are joined by a linker sequence. See, for example, Ahmad (2012) Clinical and Developmental Immunology Article ID 980250, herein specifically incorporated by reference. In principle, there are no particular limitations to the length and/or amino acid composition of the linker peptide joining the VH and VL sequences. In some embodiments, any arbitrary single-chain peptide including about 1 to 100 amino acid residues (e.g., 2, 3, 4, 5, 6, 7, 8, 9, 10, 11, 12, 13, 14, 15, 16, 17, 18, 19, 20, etc. amino acid residues) can be used as a peptide linker. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues.

The transmembrane domain may be derived either from a natural or a synthetic source. Where the source is natural, the domain may be derived from any membrane-bound or transmembrane protein. In some embodiments, the transmembrane domain comprises at least the stalk and/or transmembrane region(s) of CD8, CD28, Megf10, FcRγ, Bai1, MerTK, TIM4, Stabilin-1, Stabilin-2, RAGE, CD300f, integrin subunit av, Integrin subunit B5, CD36, LRP1, SCARF1, C1Qa, Axl, CD45, and/or CD86. In some embodiments, the CAR transmembrane domain is derived from a type I membrane protein, such as, but not limited to, CD35, CD4, CD8, or CD28. In other embodiments, the transmembrane domain is synthetic, in which case it will include predominantly hydrophobic residues such as leucine, isoleucine, valine, phenylalanine, tryptophan, and alanine. In some embodiments, a triplet of phenylalanine, tryptophan and valine will be inserted at each end of a synthetic transmembrane domain.

In some embodiments, the CAR further comprises one or more linkers/spacers. For example, an extracellular spacer region may link the antigen binding domain to the transmembrane domain and/or an intracellular spacer region may link an intracellular signaling domain to the transmembrane domain. A spacer (linker) region linking the antigen binding domain to the transmembrane domain should be flexible enough to allow the antigen binding domain to orient in different directions to facilitate antigen recognition.

Various types of linkers may be used in the CARs described herein. In some embodiments, the linker includes a peptide linker/spacer sequence. In some embodiments, the spacer comprises the hinge region from an immunoglobulin, e.g., the hinge from any one of IgG1, IgG2a, IgG2b, IgG3, IgG4, particularly the human protein sequences. Alternatives include the CH2CH3 region of immunoglobulin and portions of CD3. For many scFv based constructs, an IgG hinge is effective.

In principle, there are no particular limitations to the length and/or amino acid composition of a linker peptide sequence. In some embodiments, a linker peptide sequence comprises about 1 to 100 amino acid residues, including any number of residues within this range such as 1, 2, 3, 4, 5, 6, 7, 8, 9, 10, 11, 12, 13, 14, 15, 16, 17, 18, 19, 20, 30, 40, 50, 60, 70, 80, 90, or 100 amino acid residues. In some embodiments, the linker peptide sequence includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the linker peptide sequence includes about 1 to 10, about 5 to 15, about 10 to 20, about 15 to 25, about 20 to 40, about 30 to 50, about 40 to 60, about 50 to 70 amino acid residues. In some embodiments, the linker peptide sequence includes about 40 to 70, about 50 to 80, about 60 to 80, about 70 to 90, or about 80 to 100 amino acid residues. In some embodiments, the linker peptide sequence includes about 1 to 10, about 5 to 15, about 10 to 20, about 15 to 25 amino acid residues. In some embodiments, the linker peptide sequence may include up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular engulfment signaling domain or extracellular antigen binding domain of the CAR. In some embodiments the linker comprises the amino acid sequence (G₄S)ₙ where n is 1, 2, 3, 4, 5, etc., and in some embodiments, n is 3.

A cytoplasmic signaling domain, such as those derived from the T cell receptor ζ-chain, is employed as part of the CAR in order to produce stimulatory signals for T lymphocyte proliferation and effector function following engagement of the chimeric receptor with the target antigen. Endodomains from co-stimulatory molecules may be included in the cytoplasmic signaling portion of the CAR.

The term “co-stimulatory domain”, refers to a stimulatory domain, typically an endodomain, of a CAR that provides a secondary non-specific activation mechanism through which a primary specific stimulation is propagated. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. Nat Rev Immunol (2013) 13(4):227-42, the disclosure of which are incorporated herein by reference in their entirety. Non-limiting examples of suitable co-stimulatory polypeptides include, but are not limited to, 4-1BB (CD137), CD28, ICOS, OX-40, BTLA, CD27, CD30, GITR, and HVEM.

The term “co-inhibitory domain” refers to an inhibitory domain, typically an endodomain, derived from a receptor that provides secondary inhibition of primary antigen-specific activation mechanisms which prevents co-stimulation. Co-inhibition, e.g., T cell co-inhibition, and the factors involved have been described in Chen & Flies. Nat Rev Immunol (2013) 13(4):227-42 and Thaventhiran et al. J Clin Cell Immunol (2012) S12. In some embodiments, co-inhibitory domains homodimerize. A co-inhibitory domain can be an intracellular portion of a transmembrane protein. Non-limiting examples of suitable co-inhibitory polypeptides include, but are not limited to, CTLA-4 and PD-1.

A first-generation CAR transmits the signal from antigen binding through only a single signaling domain, for example a signaling domain derived from the high-affinity receptor for IgE FcεRIγ, or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding.

Second-generation CARs include a co-stimulatory signal in addition to the CD3ζ signal. Coincidental delivery of the delivered co-stimulatory signal enhances cytokine secretion and antitumor activity induced by CAR-transduced T-cells. The co-stimulatory domain will usually be membrane proximal relative to the CD3ζ domain. Third-generation CARs include a tripartite signaling domain, comprising for example a CD28, CD3ζ, OX40 or 4-1BB signaling region. In fourth generation, or “armored car” CAR-T cells, CAR-T cells are further genetically modified to express or block molecules and/or receptors to enhance immune activity.

CAR variants include split CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

CAR variants also include bispecific or tandem CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. Tandem CARs (TanCAR) mediate bispecific activation of T cells through the engagement of two chimeric receptors designed to deliver stimulatory or costimulatory signals in response to an independent engagement of two different tumor associated antigens. iCARs use the dual antigen targeting to shout down the activation of an active CAR through the engagement of a second suppressive receptor equipped with inhibitory signaling domains

The dual recognition of different epitopes by two CARs diversely designed to either deliver killing through ζ-chain or costimulatory signals, e.g., through CD28 allows a more selective activation of the reprogrammed T cells by restricting Tandem CAR's activity to cancer cell expressing simultaneously two antigens rather than one. The potency of delivered signals in engineered T cells will remain below threshold of activation and thus ineffective in absence of the engagement of costimulatory receptor. The combinatorial antigen recognition enhances selective tumor eradication and protects normal tissues expressing only one antigen from unwanted reactions.

Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

An ABD can be provided as a “chimeric bispecific binding member”, i.e., a chimeric polypeptide having dual specificity to two different binding partners (e.g., two different antigens). Non-limiting examples of chimeric bispecific binding members include bispecific antibodies, bispecific conjugated monoclonal antibodies (mab) 2, bispecific antibody fragments (e.g., F (ab) 2, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann. MAbs. (2012) 4(2):182-197; Stamova et al. Antibodies 2012, 1(2), 172-198; Farhadfar et al. Leuk Res. (2016) 49:13-21; Benjamin et al. Ther Adv Hematol. (2016) 7(3):142-56; Kiefer et al. Immunol Rev. (2016) 270(1):178-92; Fan et al. J Hematol Oncol. (2015) 8:130; May et al. Am J Health Syst Pharm. (2016) 73(1):e6-e13; the disclosures of which are incorporated herein by reference in their entirety.

In some instances, a chimeric bispecific binding member may be a bispecific T cell engager (BITE). A BiTE is generally made by fusing a specific binding member (e.g., a scFv) that binds an antigen to a specific binding member (e.g., a scFv) with a second binding domain specific for a T cell molecule such as CD3.

In some instances, a chimeric bispecific binding member may be a CAR-T cell adapter. As used herein, by “CAR-T cell adapter” is meant an expressed bispecific polypeptide that binds the antigen recognition domain of a CAR and redirects the CAR to a second antigen. Generally, a CAR-T cell adapter will have two binding regions, one specific for an epitope on the CAR to which it is directed and a second epitope directed to a binding partner which, when bound, transduces the binding signal activating the CAR. Useful CAR-T cell adapters include but are not limited to e.g., those described in Kim et al. J Am Chem Soc. (2015) 137 (8): 2832-5; Ma et al. Proc Natl Acad Sci USA. (2016) 113 (4):E450-8 and Cao et al. Angew Chem Int Ed Engl. (2016) 55 (26): 7520-4; the disclosures of which are incorporated herein by reference in their entirety.

Effector CAR-T cells include autologous or allogeneic immune cells having cytolytic activity against a target cell. In some embodiments, a T cell is engineered to express a CAR. The term “T cells” refers to mammalian immune effector cells that may be characterized by expression of CD3 and/or a T cell antigen receptor.

In some embodiments, the CAR-T cells are engineered from a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42 (4): 626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344 (6184) 641-645, “Cancer immunotherapy based on mutation-specific CD4+ T cells in a patient with epithelial cancer”.

In other embodiments, the engineered T cell is allogeneic with respect to the individual that is treated, e.g. see clinical trials NCT03121625; NCT03016377; NCT02476734; NCT02746952; NCT02808442. See for review Graham et al. (2018) Cells. 7 (10) E155. In some embodiments an allogeneic engineered T cell is fully HLA matched. However not all patients have a fully matched donor and a cellular product suitable for all patients independent of HLA type provides an alternative. A universal ‘off the shelf’ CAR-T cell product provides advantages in uniformity of harvest and manufacture.

Allogeneic T cells can be genetically modified to reduce graft versus host disease. For example, the TCRαβ receptor can be knocked out by different gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by the CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies CAR-T cells to express an inhibitor of TCRαβ signaling, for example using a truncated form of CD3ζ as a TCR inhibitory molecule.

Allogeneic T cells may be administered in combination with intensification of lymphodepletion to allow CAR-T cells to expand and clear malignant cells prior to host immune recovery, e.g., by administration of Alemtuzumab (monoclonal anti-CD52), purine analogs, etc. The allogeneic T cells may be modified for resistance to Alemtuzumab, and currently in clinical trials. Gene editing has also been used to prevent expression of HLA class I molecules on CAR-T cells, e.g. by deletion of β2-microglobulin, see NCT03166878.

In addition to modifying T cells, induced pluripotent stem (iPS) CAR-T cells can provide a source of allogeneic CAR-T cells. For example, transducing donor T cells with reprogramming factors can restore pluripotency, and are then re-differentiated to T effector cells.

T cells for engineering as described above collected from a subject or a donor may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS).

The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

Expression construct: The construct (e.g., CAR, antibody, scFv, etc.) coding sequence may be introduced on an expression vector into a cell to be engineered. For example, a CAR coding sequence may be introduced into the site of the endogenous T cell receptor, e.g., TRAC gene, e.g., using CRISPR technology (see, for example Eyquem et al. (2017) Nature 543:113-117; Ren et al. (2017) Protein & Cell 1-10; Ren et al. (2017) Oncotarget 8(10): 17002-17011). A CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating viruses, such as adenovirus and adeno-associated virus (AAV), has also been reported. Recent discoveries of smaller Cas proteins have enabled and enhanced the combination of this technology with vectors that have gained increasing success for their safety profile and efficiency, such as AAV vectors.

By “genetically engineered”, it is intended to mean that the genome of the cell has been manipulated to express an expression product that is not normally naturally expressed by the cell. Examples of cells that have been genetically engineered include chimeric antigen receptor (CAR)-T cells that are T-cells that have been genetically engineered to express a CAR, and cells that are engineered to express a binding-triggered transcriptional switch such as a synNotch receptor.

By “binding-triggered transcriptional switch” or “BTSS”, it is intended to mean a synthetic modular polypeptide or system of interacting polypeptides having an extracellular domain that includes a second member of a specific binding pair that binds a first member of the specific binding pair (e.g., an antigen), a binding-transducer and an intracellular domain. Upon binding of the first member of the specific binding pair to the BTTS the binding signal is transduced to the intracellular domain such that the intracellular domain becomes activated and performs a function, e.g., transcription activation, within the cell that it does not perform in the absence of the binding signal.

Examples of BTSS include the synNotch system, the MESA system, the TANGO system, the A2 Notch system, etc. The synNotch receptor may be for example as described in U.S. Pat. No. 9,670,281 and described in more detail below. The MESA system may be as described in WO 2018/081039 A1 and comprises a self-containing sensing and signal transduction system, such that binding of a ligand (first member of the specific binding pair) to the receptor (second member of the specific binding pair) induces signaling to regulate expression of a target gene. In the MESA system, binding of the ligand to the receptor induces dimerization that results in proteolytic trans-cleavage of the system to release a transcriptional activator previously sequestered at the plasma membrane. The TANGO system may be as described in Barnea et al., 2008 Proc. Natl. Acad. Sci. U.S.A., 105(1): 64-9. Briefly, the TANGO system sequesters a transcription factor to the cell membrane by physically linking it to a membrane-bound receptor (e.g., GPCRs, receptor kinases, Notch, steroid hormone receptors, etc.). Activation of the receptor fusion results in the recruitment of a signaling protein fused to a protease that then cleaves and releases the transcription factor to activate genes in the cell. The A2 Notch system may be as described in WO 2019099689 A1. Briefly, the A2 Notch system incorporates a force sensor cleavage domain which, upon cleavage induced upon binding of a ligand to the receptor, releases the intracellular domain into the cell.

In certain embodiments, the second binding member may be present on the surface of a genetically engineered cell, such as, a cell expressing a BTTS and a CAR under the control of the BTTS. In certain embodiments, the second binding member may be present on the surface of a genetically engineered cell, such as, a cell expressing the BTTS and a CAR under control of the BTTS.

In certain cases, the first binding member may bind to a synNotch receptor as described in U.S. Pat. No. 9,670,281. For example, the synNotch receptor may include an extracellular domain that includes the second binding member, where the second binding member is a single-chain Fv (scFv) or a nanobody and the first binding member present on the particles is an antigen to which the single-chain Fv (scFv) or a nanobody binds. In certain cases, the second binding member may be an anti-CD19, anti-mesothelin, anti-GFP antibody, scFv, or a nanobody and the first binding member may be CD19, mesothelin, GFP, respectively.

In certain embodiments, the BTTS is a chimeric Notch polypeptide comprising, from N-terminus to C-terminus and in covalent linkage: a) an extracellular domain comprising the second member of the specific-binding pair that is not naturally present in a Notch receptor polypeptide and that specifically binds to the first member of the specific-binding pair; b) a Notch regulatory region comprising a Lin 12-Notch repeat, an S2 proteolytic cleavage site, and a transmembrane domain comprising an S3 proteolytic cleavage site; c) an intracellular domain comprising a transcriptional activator or a transcriptional repressor that is heterologous to the Notch regulatory region and replaces a naturally-occurring intracellular Notch domain, wherein binding of the first member of the specific-binding pair to the second member of the specific-binding pair induces cleavage at the S2 and S3 proteolytic cleavage sites, thereby releasing the intracellular domain; and a transcriptional control element, responsive to the transcriptional activator, operably linked to a nucleotide sequence encoding a chimeric antigen receptor (CAR). In certain cases, the cell may be a T-cell, such as, those described in U.S. Pat. No. 9,670,281, which is herein incorporated by reference.

### Synthetic CAR-Antigen Presenting Particles

Synthetic particles presenting an antigen that binds to a chimeric antigen receptor (CAR) expressed on a cell are provided. Such particles are termed CAR-antigen presenting particles (CAPP). In certain embodiments the synthetic particle is a polymeric particle, a magnetic particle, a gold particle, or a liposome. Synthetic CAR-antigen presenting particles can be used to present an antigen to a CAR-T cell in vitro, ex vivo, or in vivo. As further described herein and without wishing to be bound by theory, synthetic CAR-antigen presenting particles are believed to exhibit certain advantages in activating CAR-T cells as compared to non-synthetic particles (e.g., cells, such as antigen presenting cells) presenting CAR antigens.

The CAR antigen may be presented on the surface of the synthetic particle through any suitable means. For example, this may involve the use of a polymer such as PEG (polyethylene glycol) with functional groups (e.g., NH₂—, —SH, N-hydroxysuccinimide (NHS), MAL, azide, alkyne, dibenzylcyclooctyne (DBCO), epoxy, aldehyde, biotin, avidin, streptavidin, etc.) on both ends. The PEG may have a molecule weight of between 50-10,000 Da. One functional group may interact with the synthetic particle and the other functional group may interact with the antigen. Also contemplated are the use of magnetic beads functionalized with avidin or biotin. Biotinylated CAR antigens can be added to a synthetic particle that includes streptavidin on the surface. In some cases, click chemistry may be used for attachment of the CAR antigen to the surface of a synthetic particle.

In some embodiments, the synthetic CAR-antigen presenting particles comprise a nucleic acid-scaffold platform for surface functionalization with an antigen of interest. Surface functionalization is accomplished using a nucleic acid-antigen conjugate, which is non-covalently attached to the surface of the particle by hybridization of the conjugate nucleic acid with a scaffold nucleic acid. Additionally, the nucleic acid-scaffold can be functionalized with antibodies, costimulatory ligands, cytokines, and/or other immunomodulatory molecules, a binding member of a BTTS, a self-peptide, and/or other molecules using nucleic acid conjugates thereof that hybridize to the scaffold nucleic acids.

In certain embodiments, the synthetic particle is a polymeric particle. In some embodiments, the CAR-antigen presenting particle comprises i) a polymeric core; ii) a nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; and iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR induces proliferation of the CAR-T cell.

The nucleic acid-polymer conjugate may be formed using the same polymer as that used to form the polymeric core or by using a different polymer. In certain embodiments, the polymer of the nucleic acid-polymer conjugate may associate non-covalently with the polymer or polymers to form the polymeric core such that the nucleic acid-polymer conjugate acts as a surfactant to produce the particles where the hydrophilic nucleic acid is displayed on the outside of the particles while the hydrophobic polymer in the conjugate is inside the particle. Thus, the nucleic acid-polymer conjugate serves as a surfactant for formation of particles from a mixture of nucleic acid-polymer conjugates and polymers.

The nucleic acid in the nucleic acid-polymer conjugate may be a single stranded nucleic acid composed of deoxyribonucleotides or ribonucleotides of a length of at least 5 bases and up to 200 bases. In certain embodiments, the nucleic acid may be 5-100 bases in length. In certain embodiments, the nucleic acid may be 10-25 bases in length and may be a polymer of deoxyribonucleotides. The nucleic acid may be conjugated to the polymer via a covalent bond. For example, the nucleic acid may be modified on its 3′-end or 5′-end by attachment of a reactive group which may react with a suitably modified polymer to form a covalent bond between the nucleic acid and the polymer. In certain embodiments, the nucleic acid may be modified to include a thiol group at the 3′-end and the polymer may be modified to include a maleimide group which may react to covalently attach the nucleic acid to the polymer.

In certain embodiments, the polymer used to form the polymeric particle may be any polymer, e.g., a biodegradable and biocompatible polymer. In certain cases, the polymer may be polylactic acid (PLA), polyglycolic acid (PGA), polyethylene glycol (PEG), and/or poly(e-caprolactone) (PCL). In another aspect, the polymer may be a copolymer such as poly(lactide-co-glycolide) (PLGA) or poly(lactide-co-glycolide) poly(e-caprolactone) (PLGA/PCL). embodiments, the polymer used to form the polymeric particle may be a combination of one or more of PLA, PGA, PEG, PCL, PLGA, and PLGA/PCL. In certain embodiments, the polymeric core of the polymeric particle comprises PLGA and PLA. In certain cases, the polymeric core of the polymeric particle comprises PLGA, PEG and PLA.

In certain cases, the polymer in the nucleic acid-polymer conjugate may be a block polymer. In certain embodiments, the polymer in the nucleic acid-polymer conjugate may be a block copolymer. In certain embodiments, the polymer in the nucleic acid-polymer conjugate may be a block copolymer of PLGA and PEG (PLGA-block-PEG). In certain embodiments, the polymer in the nucleic acid-polymer conjugate may a block copolymer of PLA and PEG (PLA-block-PEG). In certain cases, the PLGA in the block copolymer may range in molecular weight from 8 kDa-30 kDa (e.g. 10 kDa) and that of the PEG may range from 3 kDa-7 kDa (e.g. 5 kDa). In certain embodiments, the polymer in the nucleic acid-polymer conjugate may be selected such that the length of the polymer is sufficient to be inserted into the polymer core for stable association with the polymers in the core. In some cases, the polymer in the nucleic acid-polymer conjugate is non-covalently associated with the polymers forming the core of the particle. Such a non-covalent association may be via a hydrophobic interaction or via van der Walls forces. In some cases, the polymer in the nucleic acid-polymer conjugate is covalently associated with the polymers forming the core of the particle, e.g., via polymerization.

The formation of the core of the polymeric particle and association with a nucleic acid-polymer conjugate may occur simultaneously such that a plurality of hydrophobic polymers may associate with each other and away from a hydrophilic environment and may form particles that are surrounded by the nucleic acid-polymer conjugates which form an interface between the hydrophobic core and the hydrophilic environment with the polymer part of the nucleic acid-polymer conjugates interacting with the core and the nucleic acid part of the nucleic acid-polymer conjugates interacting with the hydrophilic environment. The polymers in the core may polymerize to form a stable particle which may be associated covalently (e.g., polymerized) or non-covalently (e.g., hydrophobic interaction) with the polymer in the nucleic acid-polymer conjugates.

The term particle or polymeric particle are used interchangeably to refer to one or a plurality of such particles. The polymeric particles may be substantially spherical, such as, spherical, oval, semi-spherical, hemispherical, an irregular sphere with flattened sections or concave or convex sections, semi-oval, an irregular oval with flattened sections or concave or convex sections.

The diameter of the particle refers to length from one end of the particle to the diametrically opposite end and may range from nanometers to micrometers. The size of the particles may be controlled by the amount and/or molecular weight of the polymer(s) used to form the particle. In some embodiments, the diameter of the particles may range from 1-50 μm, e.g., 1-40 μm, 1-30 μm, 1-20 μm, 1-5 μm, 1-4 μm, 1-3 μm, or 1-2 μm. In particular embodiments, the diameter of the particles range from 1-2 μm. In some embodiments, the diameter of the particles may range from 50-1000 nm, e.g., 50-1000 nm, 50-800 nm, 50-500 nm, or 100-500 nm.

Polymeric particles described herein may be made from biodegradable and substantially non-toxic polymers. The term “biodegradable polymer” refers to a polymer or polymers which degrade in vivo. A biodegradable polymer may be a homopolymer, a copolymer, or a polymer comprising more than two different polymeric units. The polymeric particles may be substantially degraded 100 days, 30 days, 10 days, or 3 days, e.g., 3-100 days, 3-10 days, 3-4 days, after being administered to a subject in need thereof. In certain embodiments, the biodegradability of the particles may be tuned based on the desired residence time in vivo. For example, the molecular weight of the polymer used to form the particles may be selected based on the desired in vivo half-life. A lower molecular weight polymer may be selected for forming particles having a lower in vivo half-life and vice versa.

In certain embodiments, the molecular weight of polymers used for generating the particles may be less than 100 kDa, e.g., less than 90 kDa, less than 80 kDa, less than 70 kDa, less than 60 kDa, less than 50 kDa, such as, 5-100 kDa, 15-90 kDa, 20-80 kDa, 30-70 kDa, 30-60 kDa, or 30-50 kDa.

In certain embodiments, the molecular weight of polymers used to form the single stranded nucleic acid-polymer conjugate may be different from the molecular weight of polymers used to form the core of the particle. For example, the molecular weight of polymers used for generating the particles may be less than 100 kDa, e.g., less than 90 kDa, less than 80 kDa, less than 70 kDa, less than 60 kDa, less than 50 kDa, such as, 5-100 kDa, 15-90 kDa, 20-80 kDa, 30-70 kDa, 30-60 kDa, or 30-50 kDa and the molecular weight of the polymer used for forming the single stranded nucleic acid-polymer conjugate may be less than 30 kDa, e.g., less than 20 kDa, less than 15 kDa, or less than 10 kDa, such as, 1 kDa-30 kDa, 3 kDa-20 kDa, 5 kDa-20 kDa, 5 kDa-15 kDa, 5 kDa-10 kDa. In certain embodiments, the polymer used for generating the single stranded nucleic acid-polymer conjugate may be a copolymer, where one of the polymers may have a molecular weight from 10 kDa-30 kDa and the other polymer may have a molecular weight from 3 kDa-8 kDa.

The antigen-nucleic acid conjugate may include a second single stranded nucleic acid covalently attached to the antigen. The second single stranded nucleic acid may be composed of deoxyribonucleotides or ribonucleotides or peptide nucleic acid and may have a length of at least 5 bases and up to 200 bases. In certain embodiments, the nucleic acid may be 5-100 bases in length. In certain embodiments, the nucleic acid may be 10-25 bases in length. In certain embodiments, the nucleic acid may be 10-25 bases or 10-20 bases in length and may be a polymer of deoxyribonucleotides. The first and second single stranded nucleic acids may include a contiguous stretch of at least 4 bases that are complementary to each other. The first and second single stranded nucleic acids may include a plurality of contiguous stretches of at least 4 bases that are complementary to each other, which contiguous stretches are separated by bases that are not complementary to each other. In certain embodiments, the region of complementarily may be about 7-20 bases, 7-19 bases, 7-18 bases, 7-10 bases, or 4-10 bases. In certain embodiments, the contiguous stretch of bases may be less than 100% complementary. For example, in a stretch of at least 7 bases, there may be at least a 99% complementary, 98% complementary, 97% complementary, 96% complementary, or 95% complementarity. In certain embodiments, the region of complementarily may be about 7-20 bases, 7-19 bases, 7-18 bases, 7-10 bases, or 4-7 bases, where all of the bases are complementary.

The nucleic acid may be conjugated to the antigen via a covalent bond directly or indirectly. In certain embodiments, the second single stranded nucleic acid is covalently attached to the antigen via a reactive group. For example, the second single stranded nucleic acid may be modified to include an amino group at the 3′-end and the first binding member may be modified to include a carboxyl group which may react to covalently attach the nucleic acid to the antigen or the second single stranded nucleic acid may be modified to include a thiol group at the 3′-end and antigen may be modified to include a maleimide group which may react to covalently attach the nucleic acid to antigen. In some embodiments, covalent attachment between the second single stranded nucleic acid and antigen may be achieved indirectly via a linker. For example, the second single stranded nucleic acid may be modified to include an amino group at the 3′-end which reacts with a carboxyl group in the linker, the linker may further include a maleimide group to react with a thiol group in the antigen.

The antigen presented on the surface of the synthetic antigen-presenting particle specifically binds to a chimeric antigen receptor expressed on a CAR-T cell. In some embodiments, the antigen is a tumor-specific antigen or a tumor-associated antigen expressed on a cancerous cell, wherein the antigen is used to activate a CAR-T cell designed for therapeutic use against a cancerous cell. Exemplary tumor-specific antigens and tumor-associated antigens include, without limitation, oncogene protein products, mutated or dysregulated tumor suppressor proteins, oncovirus proteins, oncofetal antigens, mutated or dysregulated differentiation antigens, overexpressed or aberrantly expressed cellular proteins (e.g., mutated or aberrantly expressed growth factors, mitogens, receptor tyrosine kinases, cytoplasmic tyrosine kinases, serine/threonine kinases and their regulatory subunits, G proteins, and transcription factors), and altered cell surface glycolipids and glycoproteins on cancerous cells. For example, tumor-specific antigens and tumor-associated antigens may include without limitation, dysregulated or mutated RAS, WNT, MYC, ERK, TRK, CTAG1B, MAGEA1, Bcr-Abl, p53, c-Sis, epidermal growth factor receptor (EGFR), platelet-derived growth factor receptor (PDGFR), vascular endothelial growth factor receptor (VEGFR), HER2/neu, Src-family, Syk-ZAP-70 family proteins, and BTK family of tyrosine kinases, Abl, Raf kinase, cyclin-dependent kinases, alphafetoprotein (AFP), carcinoembryonic antigen (CEA), CA-125, MUC-1, epithelial tumor antigen (ETA), tyrosinase, melanoma-associated antigen (MAGE), and other abnormal or dysregulated proteins expressed on cancerous cells.

In certain embodiments, the antigen that binds to a chimeric antigen receptor expressed on a CAR-T cell is CD19, HER2, epidermal growth factor receptor (EGFR), green fluorescent protein (GFP), fluorescein isothiocyanate (FITC), CD20, CD38, CD30, CA125, MUC-1, prostate-specific membrane antigen (PSMA), CD44 surface adhesion molecule, mesothelin, carcinoembryonic antigen (CEA), EGFRvIII, vascular endothelial growth factor receptor-2 (VEGFR2), high molecular weight-melanoma associated antigen (HMW-MAA), MAGE-A1, IL-13R-a2, GD2, MET, GPC3, CD70, EphA2, EpCAM, CLDN18, or CA9. In certain embodiments, the antigen that binds to a CAR expressed on a cell is selected from the group consisting of CD19, HER2, epidermal growth factor receptor (EGFR), MET, GPC3, CD70, EphA2, EpCAM, CLDN18, BCMA, and CA9. In certain embodiments, the antigen that binds to a CAR expressed on a cell is selected from the group consisting of CD19, HER2, and epidermal growth factor receptor (EGFR).

In some embodiments, the antigen is a fibrosis antigen expressed on an activated fibroblast or fibrotic cell, wherein the antigen is used to activate a CAR-T cell designed for therapeutic use against an activated fibroblast or fibrotic cell. In some embodiments, the fibrosis antigen is fibroblast activation protein (FAP).

In some embodiments, the antigen is a B-cell antigen for autoreactive B cell removal, wherein the antigen is used to activate a CAR-T cell designed for therapeutic use against autoreactive B cells. Antigen-specific B cell targeted therapy with CAR-T cells can be used in depleting pathogenic B cells for treatment of autoimmune diseases such as, but not limited to, systemic lupus erythematosus, type 1 diabetes, multiple sclerosis, rheumatoid arthritis, hemophilia, and pemphigus vulgaris. Exemplary B cell antigens that can be targeted for depleting pathogenic B cells include, without limitation, CD19, CD20, CD22, B cell activating factor (BAFF), and A proliferation-inducing ligand (APRIL), which can be used for targeting multiple B cell subtypes.

The T cell, from which the CAR-T cell is derived, may be autologous or allogeneic. In some embodiments, the CAR-T cell is an effector T cell (e.g., a helper CD4⁺ T cell, a cytotoxic CD8⁺ T cell, a natural killer T cell, or a gamma delta T cell) or a regulatory T cell (Treg) that has been genetically modified to express a CAR.

In certain embodiments, the antigen binds to a CAR expressed on a regulatory T cell (CAR-Tregs). Such antigens may be useful for inducing proliferation of CAR-Treg cells and may have application in cellular therapy in transplantation and autoimmune diseases. Examples of CAR-Treg cells and the antigens that they have been targeted against are provided in Zhang et al. 2018, Front. Immunol., 9:2359, which is herein incorporated by reference. See, in particular, Table 1 of Zhang et al.

In certain cases, an antigen and one or more antibodies, costimulatory ligands, cytokines, and/or other immunomodulatory molecules may be attached to the surface of a particle. In some embodiments, a first nucleic acid-polymer conjugate and a second nucleic acid-polymer conjugate are used for hybridization to two different nucleic acid-biomolecule conjugates. Use of a single stranded nucleic acid to attach a biomolecule (e.g., antigen, antibodies, costimulatory ligand, cytokine, or other immunomodulatory molecule) to a nucleic acid scaffold on the surface of a particle allows the number of biomolecules and the ratio of different biomolecules attached to the particles to be controlled. For example, to obtain a 50:50 ratio, the same amounts of a first nucleic acid-polymer conjugate and a second nucleic acid-polymer conjugate may be used. Using a ratio of 10:1 of the first nucleic acid-polymer conjugate to the second nucleic acid-polymer conjugate provides particles in which 10 times the number of a biomolecule attached to the first nucleic acid-polymer compared to the number of the biomolecule attached to the second nucleic acid-polymer is present. In some embodiments, the first nucleic acid-polymer conjugate and the second nucleic acid-polymer conjugate are present at a ratio of 1:5 to 5:1, a ratio of 1:4 to 5:1, a ratio of 1:3 to 5:1, a ratio of 1:2 to 5:1, a ratio of 1:1 to 5:1, a ratio of 1:5 to 4:1, a ratio of 1:4 to 4:1, a ratio of 1:3 to 4:1, a ratio of 1:2 to 4:1, a ratio of 1:1 to 4:1, a ratio of 2:1 to 5:1, a ratio of 2:1 to 4:1.

In certain embodiments, one or more costimulatory ligands are attached to the synthetic CAR-antigen presenting particles, wherein the costimulatory ligands bind to binding members located on the surface of a CAR-T cell. Exemplary costimulatory ligands include, without limitation, an anti-CD28 antibody, CD80, CD86, an anti-41BB antibody, an anti-ICOS antibody, or an anti-OX40 antibody.

In certain embodiments, the CAR-antigen presenting particle further comprises a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

In some embodiments, the ratio of the antigen to the costimulatory ligand on the surface of the synthetic particle is 1:20 to 20:1, including any ratio within this range such 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1. In some embodiments, the ratio of the antigen to the costimulatory ligand on the surface of the synthetic particle is 9:1.

In certain embodiments, one or more cytokines are attached to the synthetic CAR-antigen presenting particles. In some embodiments, the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle. In some embodiments, the cytokine is IL-2, IL-7, or IL-15, or a combination thereof.

In some embodiments, the ratio of the antigen to the cytokine on the surface of the synthetic particle is 1:20 to 20:1, including any ratio within this range such 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1.

In certain embodiments, a CAR-T cell is contacted with a synthetic cytokine-presenting particle, wherein the synthetic cytokine-presenting particle comprises a cytokine presented on a surface of a separate synthetic particle. In some embodiments, the synthetic particle is a polymeric particle, a magnetic bead, a gold particle, or a liposome. In some embodiments, the cytokine is IL-2, IL-7, or IL-15, or a combination thereof.

In certain embodiments, a first binding member of a binding-triggered transcriptional switch (BTSS) is attached to the synthetic CAR-antigen presenting particles. A BTSS is a synthetic modular polypeptide or system of interacting polypeptides having an extracellular domain that includes a second member of a specific binding pair (e.g., expressed by a CAR-T cell) that binds a first member of the specific binding pair (e.g., presented on a particle), a binding-transducer and an intracellular domain. Upon binding of the first member of the specific binding pair to the BTTS the binding signal is transduced to the intracellular domain such that the intracellular domain becomes activated and performs a function, e.g., transcription activation, within the cell (e.g., CAR-T cell) that it does not perform in the absence of the binding signal.

Examples of BTSS include the synNotch system, the MESA system, the TANGO system, the A2 Notch system, etc. The synNotch receptor may be for example as described in U.S. Pat. No. 9,670,281 and described in more detail below. The MESA system may be as described in WO 2018/081039 A1 and comprises a self-containing sensing and signal transduction system, such that binding of a ligand (first member of the specific binding pair) to the receptor (second member of the specific binding pair) induces signaling to regulate expression of a target gene. In the MESA system, binding of the ligand to the receptor induces dimerization that results in proteolytic trans-cleavage of the system to release a transcriptional activator previously sequestered at the plasma membrane. The TANGO system may be as described in Barnea et al., 2008 Proc. Natl. Acad. Sci. U.S.A., 105 (1): 64-9. Briefly, the TANGO system sequesters a transcription factor to the cell membrane by physically linking it to a membrane-bound receptor (e.g., GPCRs, receptor kinases, Notch, steroid hormone receptors, etc.). Activation of the receptor fusion results in the recruitment of a signaling protein fused to a protease that then cleaves and releases the transcription factor to activate genes in the cell. The A2 Notch system may be as described in WO 2019099689 A1. Briefly, the A2 Notch system incorporates a force sensor cleavage domain which, upon cleavage induced upon binding of a ligand to the receptor, releases the intracellular domain into the cell. In certain embodiments, the second binding member may be present on the surface of a genetically engineered cell, such as, a CAR-T cell expressing a BTTS and a CAR under the control of the BTTS.

In certain cases, the first binding member may bind to a synNotch receptor as described in U.S. Pat. No. 9,670,281. For example, the synNotch receptor may include an extracellular domain that includes the second binding member, where the second binding member is a single-chain Fv (scFv) or a nanobody and the first binding member present on the particles is an antigen to which the single-chain Fv (scFv) or a nanobody binds. In certain cases, the second binding member may be an anti-CD19, anti-mesothelin, anti-GFP antibody, scFv, or a nanobody and the first binding member may be CD19, mesothelin, GFP, respectively.

In certain embodiments, the BTTS is a chimeric Notch polypeptide comprising, from N-terminus to C-terminus and in covalent linkage: a) an extracellular domain comprising the second member of the specific-binding pair that is not naturally present in a Notch receptor polypeptide and that specifically binds to the first member of the specific-binding pair; b) a Notch regulatory region comprising a Lin 12-Notch repeat, an S2 proteolytic cleavage site, and a transmembrane domain comprising an S3 proteolytic cleavage site; c) an intracellular domain comprising a transcriptional activator or a transcriptional repressor that is heterologous to the Notch regulatory region and replaces a naturally-occurring intracellular Notch domain, wherein binding of the first member of the specific-binding pair to the second member of the specific-binding pair induces cleavage at the S2 and S3 proteolytic cleavage sites, thereby releasing the intracellular domain; and a transcriptional control element, responsive to the transcriptional activator, operably linked to a nucleotide sequence encoding a chimeric antigen receptor (CAR). In certain cases, the cell may be a T-cell, such as, those described in U.S. Pat. No. 9,670,281, which is herein incorporated by reference.

In certain embodiments, the particles may be attached to a self-peptide in order to reduce or avoid an immune response to the particles when administered to a subject. For example, the particles may be attached to self-peptides which enable recognition of cells by phagocytes as endogenous cells that are not phagocytosed. In certain example, the self-peptide may be a human CD47 peptide, such as, those disclosed in Science. 2013 Feb. 22; 339 (6122): 971-5.

The sequences of the nucleic acids may be selected such that only complementary sequences hybridize and non-complementary sequences do not substantially hybridize. For example, only sequences that have a complementarity of at least 95% or more hybridize. Thus, the sequence of the first single stranded nucleic acid is substantially different from that of the third single stranded nucleic acid such that the second single stranded nucleic acid hybridizes only to the first single stranded nucleic acid and the fourth single stranded nucleic acid specifically hybridizes to the third single stranded nucleic acid.

In certain embodiments, the polymeric particles provided herein may additionally include biomolecules encapsulated in the polymeric core. For example, the polymeric particles may encapsulate one or more of a nucleic acid, peptide, or polypeptide. Such polymeric particles may be utilized for release of the encapsulated biomolecules over a prolonged period in vivo.

The present disclosure also provides a composition comprising the polymeric particles disclosed herein and a pharmaceutically acceptable excipient. The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds to treat or prevent the diseases, disorders and conditions as contemplated by the present disclosure. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), and N-tris [Hydroxymethyl]methyl-3-aminopropanesulfonic acid (TAPS).

### Methods of Use

The present disclosure provides methods for using the synthetic CAR-antigen presenting particles (CAPP), disclosed herein, in a variety of in vitro, ex vivo and in vivo methods. For example, the synthetic CAR-antigen presenting particles can be used to activate effector function of a CAR-T cell, stimulate proliferation of a CAR-T cell, and/or expand numbers of a CAR-T cell in culture. Generally, the subject methods comprise contacting the CAR-T cell with a CAR-antigen presenting particle, wherein binding of the antigen, presented by the particle, to the CAR activates and/or stimulates proliferation of the CAR-T cell.

One or more rounds of antigen-stimulation with CAR-antigen presenting particles can be used to expand numbers of CAR-T cells in culture. In certain embodiments, a method of culture expansion of a CAR-T cell is provided, the method comprising: (a) culturing the CAR-T cell under suitable conditions for growth of the CAR-T cell; (b) contacting the CAR-T cell with a CAR-antigen presenting particle to produce an antigen-stimulated CAR-T cell, wherein the CAR-antigen presenting particle comprises an antigen presented on the surface of a synthetic particle, wherein binding of the antigen to the CAR induces proliferation of the CAR-T cell; (c) culturing the antigen-stimulated CAR-T cell, wherein the antigen-stimulated CAR-T cell proliferates; and (d) repeating steps (b) and (c). In some embodiments, step (c) is performed in the presence of one or more cytokines that further stimulate the CAR-T cell such as interleukin-2 (IL-2), a combination of interleukin-7 (IL-7) and interleukin-15 (IL-15), or a combination of IL-2, IL-7, and IL-15.

In certain embodiments, the method of culture expansion of a CAR-T cell comprises: (a) culturing the CAR-T cell under suitable conditions for growth of the CAR-T cell; (b) contacting the CAR-T cell with a synthetic CAR-antigen presenting particle to produce an antigen-stimulated CAR-T cell, wherein the synthetic CAR-antigen presenting particle comprises: i) a polymeric core; ii) a nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; and iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR induces proliferation of the CAR-T cell; (c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and (d) repeating steps (b) and (c).

In certain embodiments, the CAR-antigen presenting particle further comprises a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

In certain embodiments, the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle. In some embodiments, the cytokine is IL-2, IL-7, or IL-15, or a combination thereof.

In certain embodiments, the method further comprises contacting the CAR-T cell with a synthetic cytokine-presenting particle, wherein the synthetic cytokine-presenting particle comprises a cytokine presented on a surface of a synthetic particle. In some embodiments, the synthetic particle is a polymeric particle, a magnetic bead, a gold particle, or a liposome. In some embodiments, the cytokine is IL-2, IL-7, or IL-15, or a combination thereof.

Step (b) may be repeated multiple times to promote expansion of the CAR-T cells until a desired number of cells is obtained. In certain embodiments, step (d) is repeated at least 1 time, at least 2 times, at least 3 times, at least 4 times, at least 5 times, at least 6 times, at least 7 times, at least 8 times, at least 9 times, at least 10 times, or more. In certain embodiments, step (b) is repeated at intervals such as every 5 to 15 days, including any interval of time within this range such as about every 5 days, every 6 days, every 7 days, every 8 days, every 9 days, every 10 days, every 11 days, every 12 days, every 13 days, every 14 days, or every 15 days.

In certain embodiments, step (b) is performed with a ratio of the synthetic CAR-antigen presenting particle to the CAR-T cell in a range of 2:1 to 20:1, 3:1 to 15:1, 4:1 to 12:1, or 5:1 to 10:1, or any ratio within these ranges such as 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1.

In certain embodiments, the total time of culture of the CAR-T cells is at least 20 days, at least 30 days, at least 40 days, at least 50 days, at least 60 days, at least 70 days, at least 80 days, at least 90 days, at least 100 days, at least 110 days, or at least 120 days or more. In some embodiments, the CAR-T cells are expanded in culture for a total time ranging from 10 days to 150 days, including any length of time in this range such as 10 days, 12 days, 14 days, 16 days, 18 days, 20 days, 25 days, 30 days, 35 days, 40 days, 45 days, 50 days, 55 days, 60 days, 65 days, 70 days, 75 days, 80 days, 85 days, 90 days, 95 days, 100 days, 105 days, 110 days, 115 days, 120 days, 125 days, 130 days, 135 days, 140 days, 145 days, or 150 days, or longer.

In certain embodiments, step (d) is performed until at least about a 10¹⁰-fold to about a 10²⁰-fold expansion of the CAR-T cells in culture is achieved from the proliferation of the CAR-T cells, including any fold expansion within this range such as about a 10¹⁰-fold, 10¹¹-fold, 10¹²-fold, 10¹³-fold, 10¹⁴-fold, 10¹⁵-fold, 10¹⁶-fold, 10¹⁷-fold, 10¹⁸-fold, 10¹⁹-fold, or 10²⁰-fold expansion of the CAR-T cells. In certain embodiments, step (d) is performed until at least about a 10¹⁵-fold expansion of the CAR-T cells in culture is achieved from the proliferation of the CAR-T cells.

In certain embodiments, the methods of the present disclosure may be used for enhancing proliferation of a CAR-T cell without significantly increasing cytokine production by the CAR-T cell and/or causing CAR-T cell exhaustion. In certain embodiments, the level of cytokines produced by the CAR-T cells followed by contact with the CAPP, described herein, is lower than the level of the cytokines produced by CAR-T cells contacted with cells, e.g., antigen presenting cells, presenting the CAR-antigen. In certain embodiments, CAR-T cells exhibit a lower exhaustion profile following contact with CAPP compared to the exhaustion profile exhibited by CAR-T cells contacted with cells, e.g., antigen presenting cells, presenting the CAR-antigen. Exemplary methods of measuring cell proliferation, cytokine production and cell exhaustion are described herein.

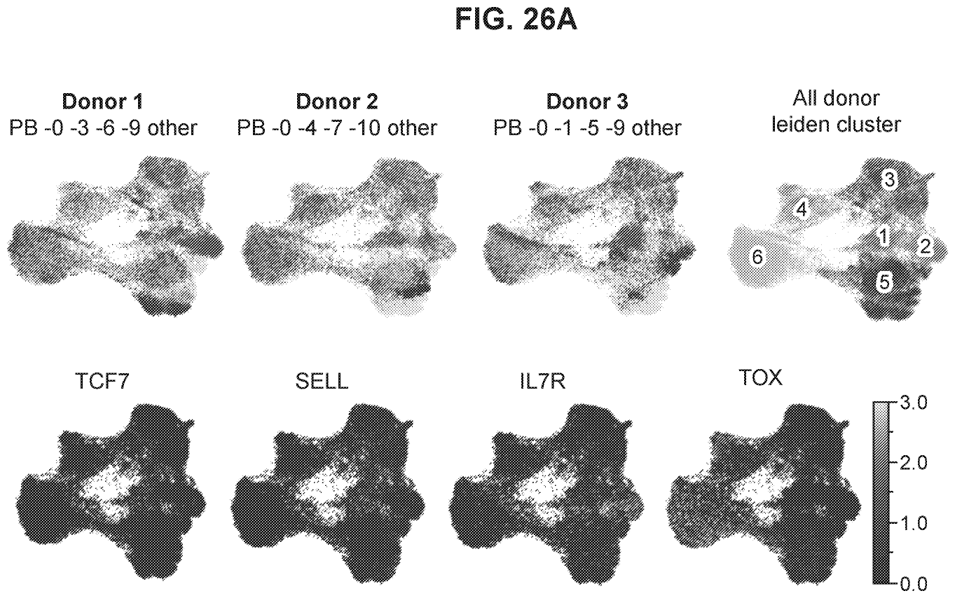

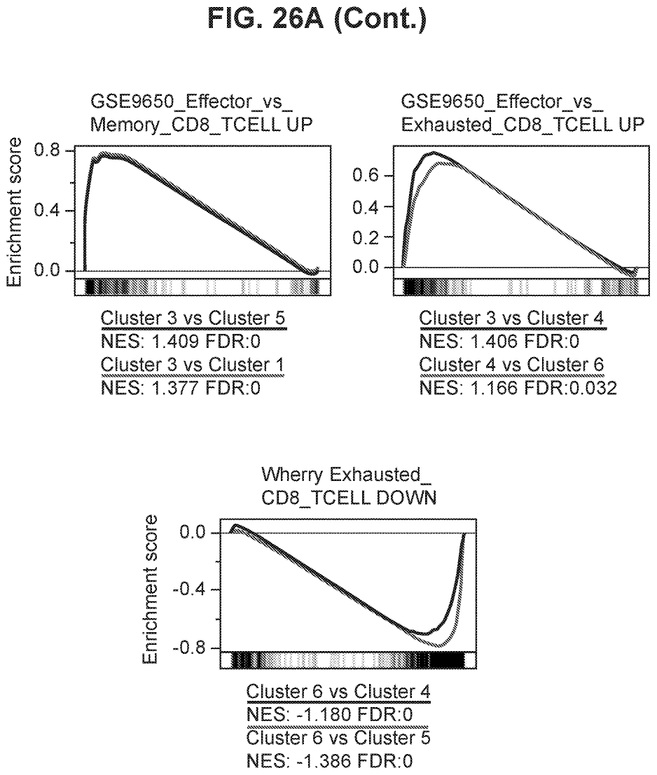

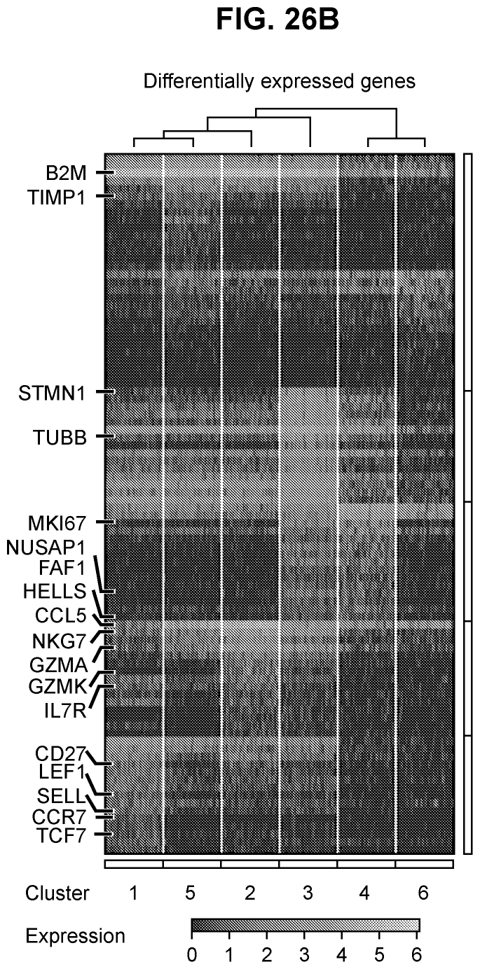

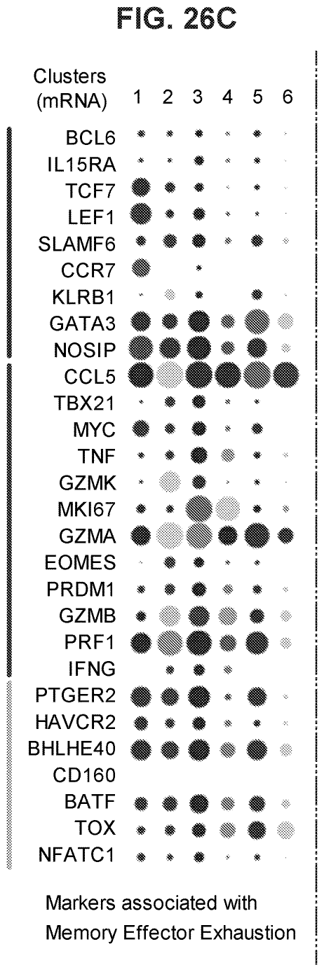

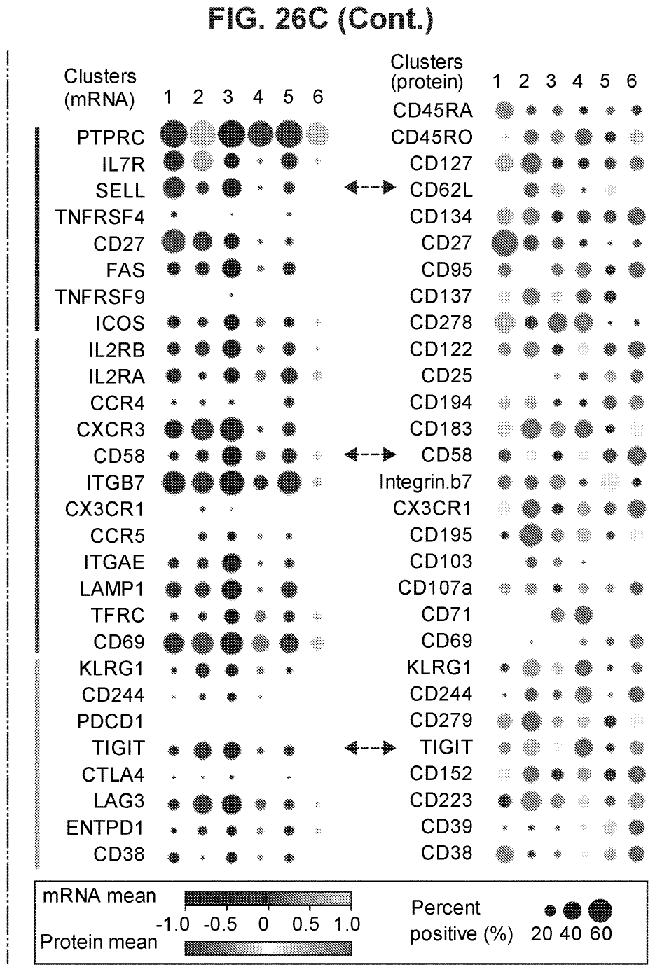

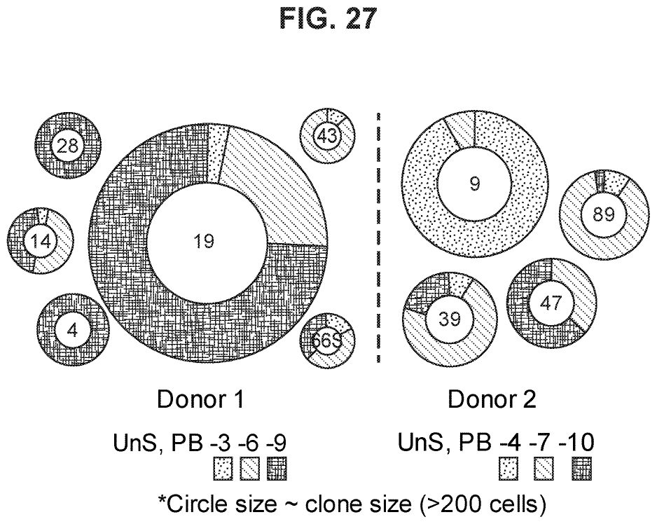

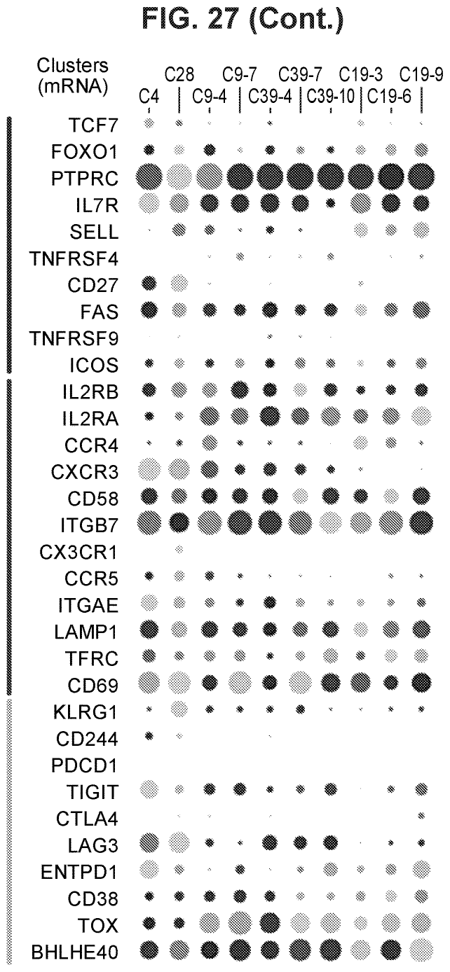

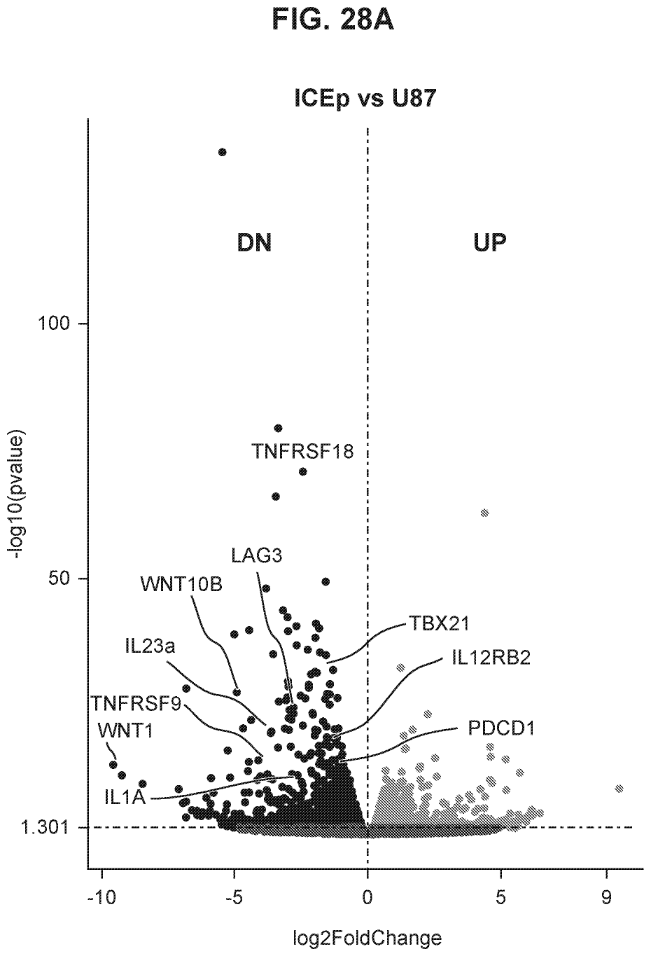

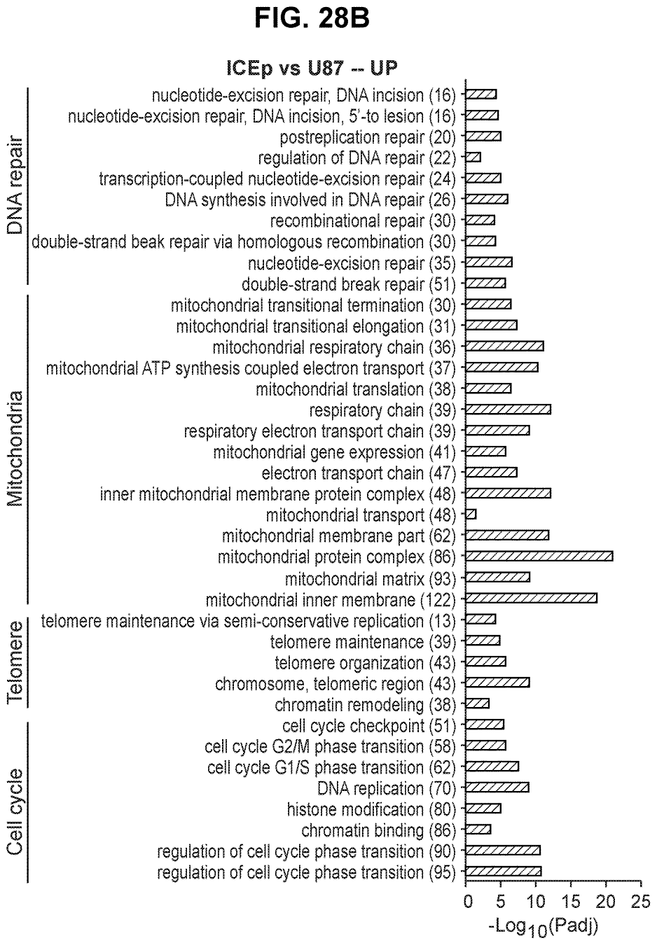

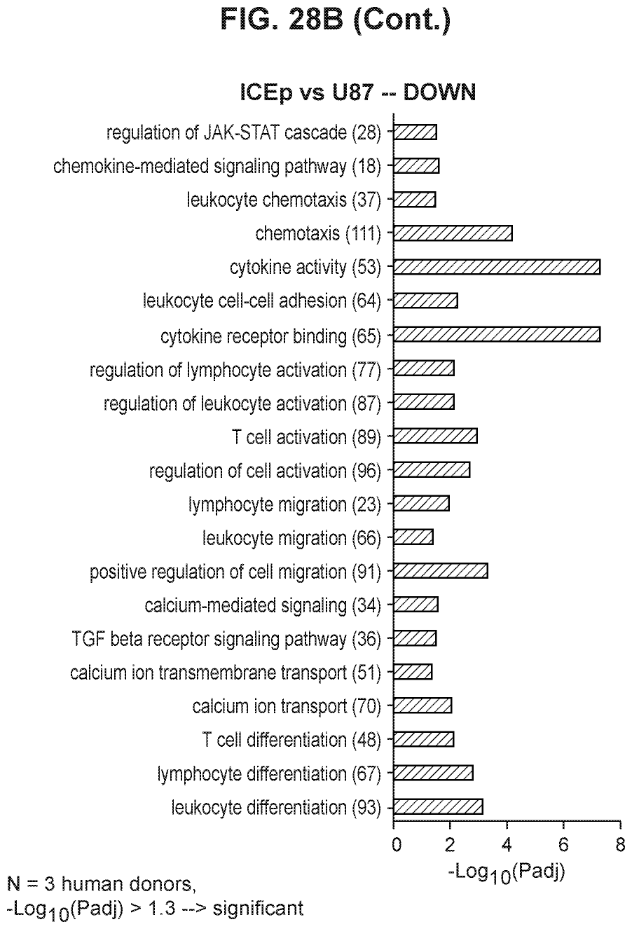

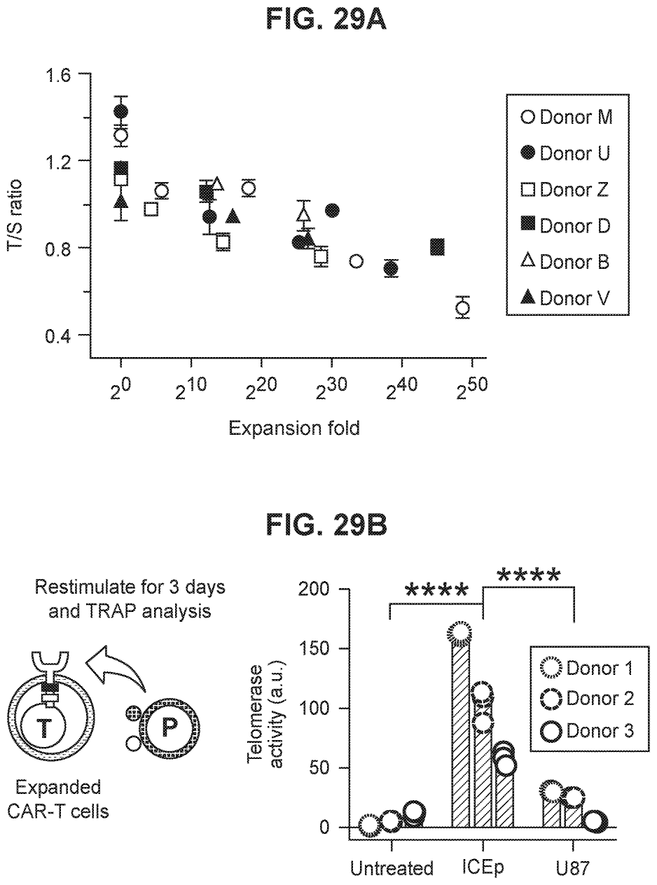

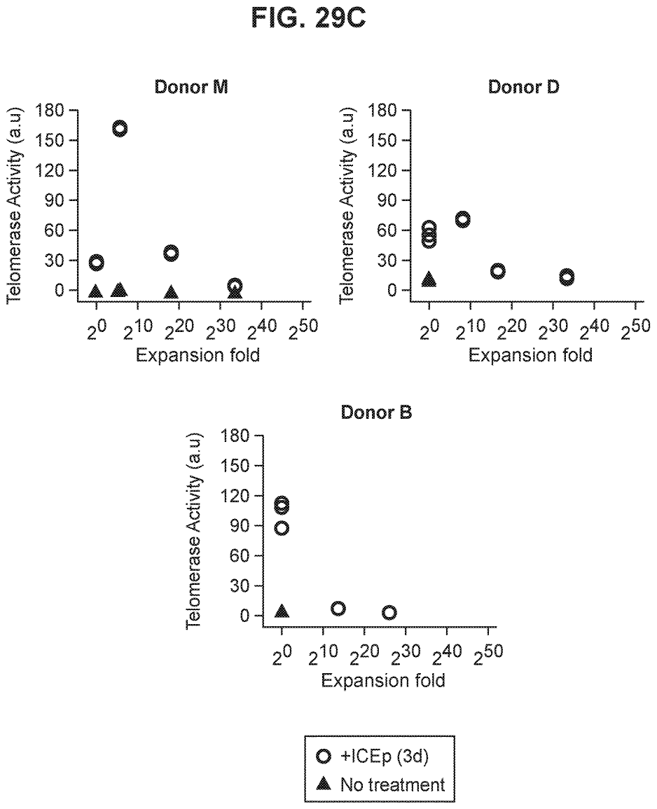

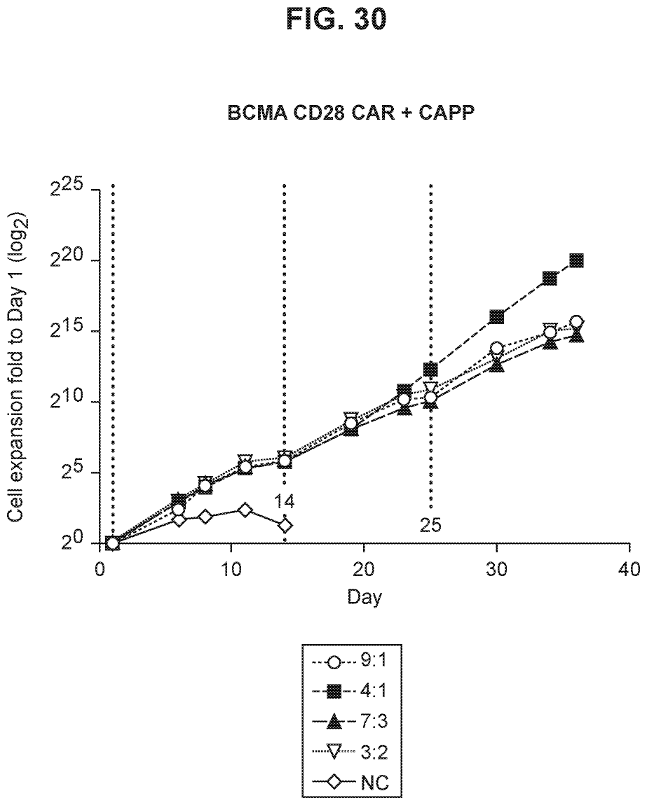

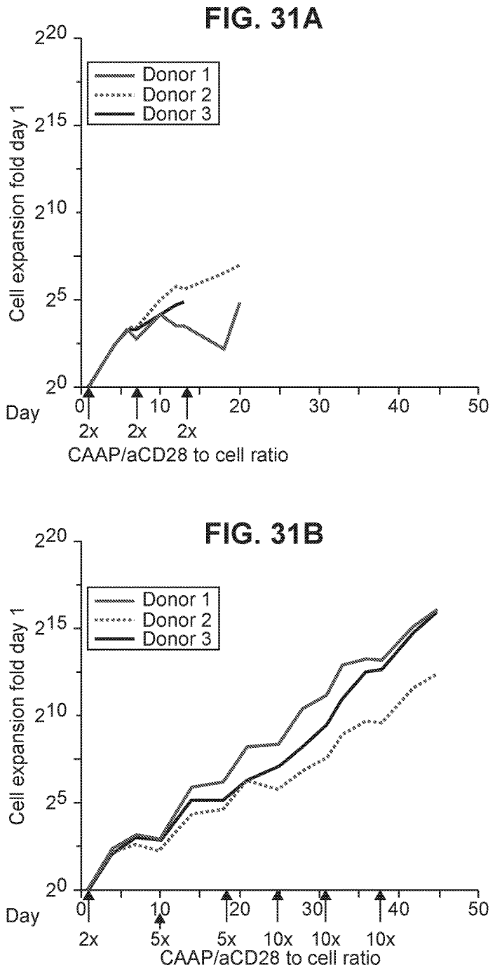

In certain embodiments, the method further comprises enriching for memory progenitor CAR-T cells, which have improved effector function. Memory progenitor CAR-T cells may be separated from other cells using any suitable cell separation technique such as, but not limited to, centrifugation-based cell separation, positive or negative selection against surface markers on cells (e.g., with antibody-coated beads), affinity chromatography, panning and immunopanning techniques, fluorescence activated cell sorting (FACS), or magnetic-activated cell sorting (MACS). Affinity reagents may be employed comprising specific receptors or ligands specific for cell surface molecules. Exemplary memory progenitor CAR-T cell markers include, without limitation, CD45RA, CD45RO, CD127, CD62L, CD134, CD27, CD95, CD137, CD278, CD195, CD38, and CD39. See also, e.g., FIG. 26C for a list of additional markers, which may be used in positive selection techniques.

The CAR-T cells may be collected in any appropriate medium that maintains the viability of the cells. Various media are commercially available and may be used according to the nature of the cells, including dMEM, HBSS, dPBS, RPMI, Iscove's medium, etc., which may be supplemented with fetal calf serum (FCS). The collected cells may be used immediately or frozen (e.g., at liquid nitrogen temperatures) prior to use.

After one or more rounds of antigen-stimulation of CAR-T cells with CAPP and/or expansion of the CAPP-stimulated CAR-T cells in culture, as described herein, the CAPP-stimulated CAR-T cells are administered to a subject in a therapeutically effective amount. The phrase “therapeutically effective amount” refers to the administration of the CAPP-stimulated CAR-T cells to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

For example, in the case of cancer, a therapeutically effective amount of the CAR-T cells provides an anti-tumor effect, as defined herein. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In some embodiments, CAR-T cells are administered to a subject who has an impaired T cell response. For example, a subject may be immunocompromised because of treatment with chemotherapeutic agents or immunosuppressive agents (e.g., glucocorticoid therapy) or immunodeficient because of a disease such as acquired immune deficiency syndrome (AIDS). For subjects with an impaired T cell response to a pathogenic antigen, a therapeutically effective amount of CAR-T cells specific for the pathogenic antigen may augment the T cell response of the subject, e.g., to suppress an infection or eliminate pathogenic or damaged cells.

In certain embodiments, the methods of using CAPP to stimulate proliferation and expansion of CAR-T cells are carried out ex vivo. In certain embodiments, the ex vivo method comprises contacting a population of T cells comprising a CAR-T cell with the CAPP, wherein the population of T cells have been obtained from the subject to be treated, then genetically modified to express a CAR. After one or more rounds of antigen-stimulation with CAPP and expansion of the CAR-T cells in culture, the autologous CAR-T cells are subsequently administered to the subject.

In certain embodiments, the methods of using CAPP to stimulate proliferation and expansion of CAR-T cells are carried out in vitro. In certain embodiments, the in vitro method comprises contacting a population of T cells comprising a CAR-T cell with the CAPP, wherein the T cells have been obtained from a donor, a culture of cells from a donor, or from established cell culture lines, then genetically modified to express a CAR. The T cells may be obtained from the same or a different species than the subject to be treated, but preferably are of the same species, and more preferably of the same immunological profile as the subject. Such cells can be obtained, for example, from a blood sample comprising T cells from a close relative or matched donor. After one or more rounds of antigen-stimulation with CAPP and expansion of the CAR-T cells in culture, the CAR-T cells may be subsequently administered to a subject.

In certain embodiments, the methods of using CAPP to stimulate proliferation and expansion of CAR-T cells are carried out in vivo. In certain embodiments, the in vivo method comprises administering the CAPP to the subject.

In the in vitro, ex vivo, or in vivo methods described herein, the subject may have cancer, wherein the CAR-antigen presenting particles, used for stimulation of the CAR-T cells, present a cancer antigen that specifically binds to the CAR expressed on the CAR-T cells. In certain embodiments, the subject has a B cell cancer such as leukemia. In certain embodiments, the leukemia is relapsed or refractory CD 19+ leukemia. In certain embodiments, the cancer is lymphoma or myeloma. In certain embodiments, the cancer is prostate cancer or glioblastoma. In certain embodiments, the subject has fibrosis, wherein the CAR-antigen presenting particles, used for stimulation of the CAR-T cells, present a fibrosis antigen that specifically binds to the CAR expressed on the CAR-T cells. In certain embodiments, the subject has an autoimmune disease, wherein the CAR-antigen presenting particles, used for stimulation of the CAR-T cells, present a B cell antigen for autoreactive B cell removal that specifically binds to the CAR expressed on the CAR-T cells. Antigen-specific B cell targeted therapy with CAR-T cells specific for B cell antigens can be used in depleting pathogenic B cells for treatment of autoimmune diseases such as, but not limited to, systemic lupus erythematosus, type 1 diabetes, multiple sclerosis, rheumatoid arthritis, hemophilia, and pemphigus vulgaris. Exemplary B cell antigens that can be targeted for depleting pathogenic B cells include, without limitation, CD19, CD20, CD22, B cell activating factor (BAFF), and A proliferation-inducing ligand (APRIL), which can be used for targeting multiple B cell subtypes. In certain embodiments, the subject is undergoing or has previously undergone CAR-T cell immunotherapy.

The present disclosure contemplates the administration of the CAPP-stimulated CAR-T cells, and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual, inhalation, local, e.g., injection directly into a target organ or tissue such as a tumor.

The present disclosure also contemplates the administration of CAR-T cells, and compositions thereof in combination with the synthetic CAR-antigen presenting particles. As used herein, “combination” is meant to include therapies that can be administered separately, for example, formulated separately for separate administration (e.g., as may be provided in a kit), and therapies that can be administered together in a single formulation (i.e., a “co-formulation”). In certain embodiments, a CAPP and a CAR-T cell as disclosed herein are administered sequentially, e.g., wherein CAPP are administered prior to or after administering the CAR-T cells. In other embodiments, the CAPP and the CAR-T cells are administered simultaneously, e.g., where CAR-T cells and CAPP are administered at or about the same time; the CAPP and CAR-T cells may be present in two or more separate formulations or combined into a single formulation (i.e., a co-formulation).

In some embodiments, the CAR-T cells may comprise a binding-triggered transcriptional switch. In some embodiments, the method may further include activating a T cell such as a T cell expressing a chimeric Notch polypeptide, as described herein. In certain embodiments, the method of the present disclosure may be used for inducing T-cell proliferation without significantly increasing cytokine production by the T cell. For example, the method may include administering a T cell expressing a chimeric Notch polypeptide and CAPP having a protein displayed on the surface, where the protein binds to the Notch polypeptide resulting in expression of a cancer associated CAR on the cell surface. The CAPP further includes an antigen that binds the cancer associated CAR, where binding of the antigen on the particle to the cancer associated CAR results in activation of the T cell in absence of significant expression of cytokines. In certain embodiments, the level of cytokines produced by the T cells in the absence of cancer cells expressing the CAR antigen is substantially lower than the level of the cytokines produced by the T cells in the presence of cancer cells expressing the CAR antigen. Thus, use of particles functionalized with both a protein that binds to the chimeric Notch polypeptide and an antigen that binds to the CAR expressed in response to the binding of the protein to the chimeric Notch polypeptide provides for proliferation of the T-cells while having a substantially lower production of cytokines by the activated T cell.

In certain aspects, contacting a CAR-T cell expressing a BTTS, e.g., a chimeric Notch receptor polypeptide, as described herein with the CAPP of the present disclosure may modulate an activity of the CAR-T cell. In some cases, release of the intracellular domain modulates proliferation of the cell or of cells surrounding the cell. In some cases, release of the intracellular domain modulates apoptosis in the cell or in cells surrounding the cell. In some cases, release of the intracellular domain induces cell death by a mechanism other than apoptosis. In some cases, release of the intracellular domain modulates gene expression in the cell through transcriptional regulation, chromatin regulation, translation, trafficking or post-translational processing. In some cases, release of the intracellular domain modulates differentiation of the cell. In some cases, release of the intracellular domain modulates migration of the cell or of cells surrounding the cell. In some cases, release of the intracellular domain modulates the expression and secretion of a molecule from the cell. In some cases, release of the intracellular domain modulates adhesion of the cell to a second cell or to an extracellular matrix. In some cases, release of the intracellular domain induces de novo expression a gene product in the cell. In some cases, where release of the intracellular domain induces de novo expression a gene product in the cell, the gene product is a transcriptional activator, a transcriptional repressor, a chimeric antigen receptor, a second chimeric Notch receptor polypeptide, a translation regulator, a cytokine, a hormone, a chemokine, or an antibody.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527.

In certain embodiments, the method may include a method of activating a T cell, e.g., a T-cell expressing CD3 and CD28. In certain embodiments, the method of the present disclosure may be used for inducing T-cell proliferation without significantly increasing cytokine production by the T cell and/or causing T-cell exhaustion. For example, the method may include contacting a T cell with a particle having first binding members (e.g., antibodies) displayed on its surface, where the first binding members bind to second binding members expressed on the surface of the T cell (e.g., CD3 and CD28), and where binding of the first binding members to the second binding members induces T-cell proliferation without significant increase in cytokine production. In certain embodiments, the level of cytokines produced by the T cells in the presence of the particles described herein is lower than the level of the cytokines produced by the T cells in the presence of non-polymeric particles (e.g., magnetic beads, gold particles, liposomes) having the same first binding members. In certain embodiments, the T-cells exhibit a lower exhaustion profile in the presence of the particles described herein compared to the exhaustion profile exhibited by T-cells in the presence of non-polymeric particles (e.g. magnetic beads, gold particles, liposomes) having the same first binding members.

Cell proliferation can be determined and quantified, for example, using a cell counter, such as is described further in the Examples herein. The levels of cytokines produced by T-cells can be determined and quantified, for example, by measuring the levels of (extracellular and/or intracellular) interferon-gamma produced by the cells, such as is further described in the Examples herein. The exhaustion profile can be determined, for example, by measuring the levels of T-cell exhaustion markers such as LAG-3, PD-1 and/or TIM-3, such as is further described in the examples. A lower exhaustion profile may be revealed by a lower expression profile of one, two or all three of LAG-3, PD-1 and TIM-3.

### Methods of Making CAR-Antigen Presenting Particles

The present disclosure also provides methods for making the CAR-antigen presenting particles disclosed herein. The method may include the formation of an emulsion to generate the polymeric particles functionalized with an antigen on a surface thereof. In certain embodiments, the method may include the formation of a double emulsion to generate polymeric particles encapsulating a biomolecule inside the particles and functionalized with an antigen on a surface thereof. Additionally, the polymeric particles can be functionalized with antibodies, costimulatory ligands, cytokines, and/or other immunomodulatory molecules, a binding member of a BTTS, a self-peptide, and/or other molecules on a surface thereof.

In certain embodiments, a method of making an antigen-presenting polymeric particle comprises covalently linking a nucleic acid to a first polymer to generate a nucleic acid-polymer conjugate, wherein the nucleic acid is a first single stranded nucleic acid; sonicating a solution that includes the nucleic acid-polymer conjugate and a second polymer to generate polymeric particles comprising a polymeric core comprising the second polymer, wherein the polymer region of the nucleic acid-polymer conjugate is non-covalently associated with the polymeric core thereby presenting the first single stranded nucleic acid on a surface of the polymeric core; attaching to the polymeric core a second single stranded nucleic acid having a sequence complementary to the first single stranded nucleic acid by hybridization; and covalently or non-covalently attaching the second single stranded nucleic acid to an antigen to generate the antigen-presenting polymeric particle.

Hybridization may be performed under conditions and for a period of time sufficient for formation of specific-base pairing between complementary bases in the nucleic acids (e.g., for formation DNA:DNA or DNA:RNA or RNA:RNA or DNA:PNA or PNA:PNA double stranded regions). In certain cases, the hybridization may be performed at a temperature of at least 30° C., such as, 30° C.-50° C., 37° C.-50° C. or 37° C.-45° C. for at least 10 min, e.g., 10 min-60 min, 10 min-45 min, or 10 min-30 min.

In certain embodiments, the method comprises covalently attaching the second single stranded nucleic acid to the antigen prior to attaching the second single stranded nucleic acid to the polymeric core.

In certain embodiments, the method comprises covalently attaching the second single stranded nucleic acid to the antigen after attaching the second single stranded nucleic acid to the polymeric core.

The single stranded nucleic acid and the first binding member may be attached directly or indirectly via a linker.

In certain embodiments, the method comprises covalently attaching the second single stranded nucleic acid to a biotin molecule and non-covalently attaching an avidin-antigen conjugate to the second single stranded nucleic acid.

In certain embodiments, the method comprises generating a plurality of nucleic acid-polymer conjugates, wherein the plurality of nucleic acid-polymer conjugates at least include a first nucleic acid-polymer conjugate comprising a first single stranded nucleic acid covalently linked to a first polymer molecule; and a second nucleic acid-polymer conjugate comprising a third single stranded nucleic acid covalently linked to a first polymer molecule, where the first single stranded nucleic acid and the third single stranded nucleic acid have different sequences. In certain cases, the plurality of nucleic acid-polymer conjugates includes at least three, at least four, at least five, or more different nucleic acid-polymer conjugates each comprising a different single stranded nucleic acid, where the sequences are sufficiently different to enable hybridization to different nucleic acids, which are in turn, attached to different molecules, e.g., to allow functionalization of the surface of the particle with antigens, antibodies, costimulatory ligands, cytokines, and/or other immunomodulatory molecules, a binding member of a BTTS, a self-peptide, and/or other molecules.

As noted herein, the plurality of nucleic acid-polymer conjugates can be included in a desired ratio. For example, the first nucleic acid-polymer conjugate and the second nucleic acid-polymer conjugate may be included in the solution at a ratio of 100:1, 50:1, 10:1, 5:1, 3:1, 2:1, 1:1, 1:2, 1:3, 1:5, 1:10, 1:50, or 1:100. In another example, the first nucleic acid-polymer conjugate, the second nucleic acid-polymer conjugate, and a third nucleic acid-polymer conjugate may be included in the solution at a ratio of 10:1:1, 5:1:1, 3:1:1, 2:1:1, 1:1:1, 1:1:2, 1:1:3, 1:1:5, or 1:1:10.

In certain embodiments an antigen-nucleic acid conjugate and a costimulatory ligand-nucleic acid conjugate may be included in the solution at a ratio of 1:20 to 20:1, including any ratio within this range such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1.

In certain embodiments an antigen-nucleic acid conjugate and an anti-CD28 antibody-nucleic acid conjugate may be included in the solution at a ratio of 4:1 to 15:1, including any ratio within this range such as 4:1, 5:1, 6:1, 7:1, 8:1, 9:1, 10:1, 11:1, 12:1, 13:1, 14:1, or 15:1. In some embodiments, the ratio of the antigen-nucleic acid conjugate to the anti-5 CD28 antibody-nucleic acid conjugate, included in the solution, is 9:1.

In certain embodiments an antigen-nucleic acid conjugate, a costimulatory ligand-nucleic acid conjugate, and a cytokine-nucleic acid conjugate may be included in the solution. In certain embodiments an antigen-nucleic acid conjugate and a cytokine-nucleic acid conjugate may be included in the solution at a ratio of 1:20 to 20:1, including any ratio within this range such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1.

The step of covalently linking a nucleic acid to a first polymer to generate a nucleic acid-polymer conjugate may be carried out using standard chemistry suitable for the reactive groups being used for the covalent attachment. Examples of suitable reactive groups include thiol-maleimide, amino and carboxyl groups, thiol and carboxyl groups, and the like. Any suitable solution may be used for forming an emulsion from the polymers to generate the polymeric particles. In certain embodiments, the solution may be substantially hydrophilic. Sonication may be carried out for a period of time and under conditions sufficient for generation of the particles.

In certain embodiments, a double emulsion procedure may be performed for encapsulating substantially hydrophilic biomolecules or amphipathic biomolecules in the polymeric core of the particles. In certain embodiments, the method of making a polymeric particle comprising peptide, polypeptide, and/or nucleic acid encapsulated in a polymeric core and a nucleic acid-polymer conjugate comprising a first single stranded nucleic acid covalently attached to a first polymer, wherein the polymer is non-covalently associated with the polymeric core thereby presenting the first single stranded nucleic acid on a surface of the polymeric core may include sonicating a solution comprising the peptide, polypeptide, and/or nucleic acid and a second polymer; adding the nucleic acid-polymer conjugate to the solution and further sonicating the solution to generate polymeric particles comprising a polymeric core comprising the second polymer and encapsulating the peptide, polypeptide, and/or nucleic acid, wherein the polymer region of the nucleic acid-polymer conjugate is non-covalently associated with the polymeric core thereby presenting the first single stranded nucleic acid on a surface of the polymeric core; attaching to the polymeric core a second single stranded nucleic acid having a sequence complementary to the first single stranded nucleic acid by hybridization; and covalently or non-covalently attaching the second single stranded nucleic acid to an antigen to generate the antigen-presenting polymeric particle.

In certain cases, the initial emulsification may be carried out in an organic solution such that hydrophilic biomolecules are distributed to the interior and surrounded by the polymer, followed by further emulsification in an aqueous phase, for example, by adding to the emulsion a solution of the nucleic acid-polymer conjugate dissolved in water, followed by sonication, resulting in insertion of the polymer region of the nucleic acid-polymer conjugate into the particles to generate the polymeric particles encapsulating the biomolecules. The functionalization of the particle to add the antigen and/or other molecules may then be carried out as outlined herein.

### Antigen-Presenting Films

Antigen-presenting films functionalized by attaching an antigen of interest to the surface of a film are also provided. Additionally, the surface of a film can be functionalized with antibodies, costimulatory ligands, cytokines, and/or other immunomodulatory molecules, a binding member of a BTTS, a self-peptide, and/or other molecules.

In certain embodiments, a film is functionalized by attaching an antigen of interest and one or more other types of biomolecules to the film, optionally wherein the number of each of the molecules and the ratio of the molecules to one another are controlled. In certain embodiments, antigen-presenting films are functionalized by attaching an antigen of interest and one or more other molecules, wherein the density of each is controlled. In certain embodiments, an antigen-presenting film may encapsulate biomolecules in addition to having an antigen and one or more biomolecules presented on the surface of the film. In certain embodiments, the film is functionalized by attaching a CAR-antigen to the surface and may have the features and properties similar to CAR-antigen presenting particles and/or used similarly in the methods of enhancing proliferation and expansion of CAR-T cells in culture as described for CAR-antigen presenting particles.

In certain embodiments, the antigen-presenting film comprises a polymeric film comprising one or more pores; a nucleic acid-polymer conjugate comprising a first single stranded nucleic acid covalently attached to a polymer, wherein the polymer is covalently or non-covalently associated with the polymeric film thereby presenting the first single stranded nucleic acid on a surface of the polymeric film; and an antigen-nucleic acid conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on a surface of the polymeric film.

In certain embodiments, the nucleic acid and/or nucleic acid-polymer conjugate may be as further described in the context of the polymeric particles herein.

In certain embodiments, the polymer used to form the polymeric film may be any polymer, e.g., a biodegradable and biocompatible polymer. In certain cases, the polymer may be polylactic acid (PLA), polyglycolic acid (PGA), poly(D,L-lactide-co-glycolide) (PLGA), polyethylene glycol (PEG), and/or poly(e-caprolactone) (PCL). In another aspect, the polymer may be a copolymer such as poly(lactide-co-glycolide) (PLGA) or poly(lactide-co-glycolide) poly(e-caprolactone) (PLGA/PCL). In certain embodiments, the polymer used to form the polymeric film may be a combination of one or more of PLA, PGA, PEG, PCL, PLGA, and PLGA/PCL. In certain embodiments, the polymeric film comprises PCL and PEG.

In certain embodiments, the polymeric film comprises pores having a diameter of between 0.1 μm to 10 μm. For example, the pore may have a diameter of between 0.1 μm to 5 μm, 0.1 μm to 3 μm, 0.1 μm to 2 μm, 0.5 μm to 10 μm, 0.5 μm to 5 μm, 0.5 μm to 3 μm, 0.5 μm to 2 μm, 1 μm to 10 μm, 1 μm to 5 μm, 1 μm to 3 μm, or 1 to 2 μm. In certain embodiments, the polymeric film comprises pores having a diameter of between 1 μm to 2 μm. In certain embodiments, the polymeric film comprises pores having a diameter of between 10 nm to 100 nm. For example, the pore may have a diameter of between 10 nm to 90 nm, 10 nm to 80 nm, 20 nm to 90 nm, 30 nm to 90 nm, 30 nm to 80 nm, 40 nm to 90 nm, 40 nm to 80 nm, 50 nm to 90 nm, or 50 nm to 80 nm.

In certain embodiments, the thickness of the polymeric film is between 0.1 μm and 100 μm. For example, the polymeric film may have a thickness of between 0.1 μm and 1 μm, 1 μm and 80 μm, 1 μm and 70 μm, 1 μm and 60 μm, 50 μm, 1 μm and 40 μm, 1 μm and 30 μm, 1 μm and 20 μm, 5 μm and 80 μm, 5 μm and 70 μm, 5 μm and 60 μm, 5 μm and 50 μm, 5 μm and 40 μm, 5 μm and 30 μm, 5 μm and 20 μm, 10 μm and 80 μm, 10 μm and 70 μm, 10 μm and 60 μm, 10 μm and 50 μm, 10 μm and 40 μm, 10 μm and 30 μm, or 10 μm and 20 μm. In certain embodiments, the polymeric film has a thickness of between 10 μm and 20 μm.

The antigen presented on the surface of the film specifically binds to a chimeric antigen receptor expressed on a CAR-T cell. In some embodiments, the antigen is a tumor-specific antigen or a tumor-associated antigen expressed on a cancerous cell, wherein the antigen is used to activate a CAR-T cell designed for therapeutic use against the cancerous cell. Exemplary tumor-specific antigens and tumor-associated antigens include, without limitation, oncogene protein products, mutated or dysregulated tumor suppressor proteins, oncovirus proteins, oncofetal antigens, mutated or dysregulated differentiation antigens, overexpressed or aberrantly expressed cellular proteins (e.g., mutated or aberrantly expressed growth factors, mitogens, receptor tyrosine kinases, cytoplasmic tyrosine kinases, serine/threonine kinases and their regulatory subunits, G proteins, and transcription factors), and altered cell surface glycolipids and glycoproteins on cancerous cells. For example, tumor-specific antigens and tumor-associated antigens may include without limitation, dysregulated or mutated RAS, WNT, MYC, ERK, TRK, CTAG1B, MAGEA1, Bcr-Abl, p53, c-Sis, epidermal growth factor receptor (EGFR), platelet-derived growth factor receptor (PDGFR), vascular endothelial growth factor receptor (VEGFR), HER2/neu, Src-family, Syk-ZAP-70 family proteins, and BTK family of tyrosine kinases, Abl, Raf kinase, cyclin-dependent kinases, alphafetoprotein (AFP), carcinoembryonic antigen (CEA), CA-125, MUC-1, epithelial tumor antigen (ETA), tyrosinase, melanoma-associated antigen (MAGE), and other abnormal or dysregulated proteins expressed on cancerous cells.

In certain embodiments, the antigen that binds to a chimeric antigen receptor expressed on a CAR-T cell is CD19, HER2, epidermal growth factor receptor (EGFR), green fluorescent protein (GFP), fluorescein isothiocyanate (FITC), CD20, CD38, CD30, CA125, MUC-1, prostate-specific membrane antigen (PSMA), CD44 surface adhesion molecule, mesothelin, carcinoembryonic antigen (CEA), EGFRvIII, vascular endothelial growth factor receptor-2 (VEGFR2), high molecular weight-melanoma associated antigen (HMW-MAA), MAGE-A1, IL-13R-a2, GD2, MET, GPC3, CD70, EphA2, EpCAM, CLDN18, or CA9. In certain embodiments, the antigen that binds to a CAR expressed on a cell is selected from the group consisting of CD19, HER2, epidermal growth factor receptor (EGFR), MET, GPC3, CD70, EphA2, EpCAM, CLDN18, BCMA, and CA9. In certain embodiments, the antigen that binds to a CAR expressed on a cell is selected from the group consisting of CD19, HER2, and epidermal growth factor receptor (EGFR).

In some embodiments, the antigen is a fibrosis antigen expressed on an activated fibroblast or fibrotic cell, wherein the antigen is used to activate a CAR-T cell designed for therapeutic use in treating fibrosis. In some embodiments, the fibrosis antigen is fibroblast activation protein (FAP).

The T cell, from which the CAR-T cell is derived, may be autologous or allogeneic. In some embodiments, the CAR-T cell is an effector T cell (e.g., a helper CD4⁺ T cell, a cytotoxic CD8⁺ T cell, a natural killer T cell, or a gamma delta T cell) or a regulatory T cell (Treg) that has been genetically modified to express a CAR.

In certain embodiments, the antigen binds to a CAR expressed on a regulatory T cell (CAR-Tregs). Such antigens may be useful for inducing proliferation of CAR-Treg cells and may have application in cellular therapy in transplantation and autoimmune diseases. Examples of CAR-Treg cells and the antigens that they have been targeted against are provided in Zhang et al. 2018, Front. Immunol., 9:2359, which is herein incorporated by reference. See, in particular, Table 1 of Zhang et al.

In certain cases, an antigen and one or more antibodies, costimulatory ligands, cytokines, and/or other immunomodulatory molecules may be attached to the surface of the film. In some embodiments, a first nucleic acid-polymer conjugate and a second nucleic acid-polymer conjugate are used for hybridization to two different nucleic acid-biomolecule conjugates. Use of a single stranded nucleic acid to attach a biomolecule (e.g., antigens, antibodies, costimulatory ligands, cytokines, and/or other immunomodulatory molecules, a binding member of a BTTS, a self-peptide, and/or other molecules) to a nucleic acid scaffold on the surface of the film allows the number of biomolecules and the ratio of different biomolecules attached to the surface of the film to be controlled. For example, to obtain a 50:50 ratio, the same amounts of a first nucleic acid-polymer conjugate and a second nucleic acid-polymer conjugate may be used. Using a ratio of 10:1 of the first nucleic acid-polymer conjugate to the second nucleic acid-polymer conjugate provides 10 times the number of a biomolecule attached to the first nucleic acid-polymer compared to the number of the biomolecule attached to the second nucleic acid-polymer on the surface of the film. In some embodiments, the first nucleic acid-polymer conjugate and the second nucleic acid-polymer conjugate are present at a ratio of 1:5 to 5:1, a ratio of 1:4 to 5:1, a ratio of 1:3 to 5:1, a ratio of 1:2 to 5:1, a ratio of 1:1 to 5:1, a ratio of 1:5 to 4:1, a ratio of 1:4 to 4:1, a ratio of 1:3 to 4:1, a ratio of 1:2 to 4:1, a ratio of 1:1 to 4:1, a ratio of 2:1 to 5:1, a ratio of 2:1 to 4:1 on the surface of a film.

In certain embodiments, the polymeric film further comprises a second nucleic acid-polymer conjugate comprising a third single stranded nucleic acid covalently attached to the polymer, wherein the polymer is non-covalently associated with the polymeric film thereby presenting the third single stranded nucleic acid on the surface of the polymeric film and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric film. In certain embodiments, the costimulatory ligand is an anti-CD28 antibody, CD80, CD86, an anti-41BB antibody, an anti-ICOS antibody, or an anti-OX40 antibody. In some embodiments, the ratio of the antigen to the costimulatory ligand on the surface of the polymeric film is 1:20 to 20:1, including any ratio within this range such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1.

In certain embodiments, the polymeric film further comprises a third nucleic acid-polymer conjugate comprising a fifth single stranded nucleic acid covalently attached to the polymer, wherein the polymer is non-covalently associated with the polymeric film thereby presenting the fifth single stranded nucleic acid on the surface of the polymeric film and a cytokine-nucleic acid conjugate comprising a sixth single stranded nucleic acid covalently attached to a cytokine, wherein the sixth single stranded nucleic acid is complementary to the fifth single stranded nucleic acid and is associated with the fifth single stranded nucleic acid via hybridization thereby presenting the cytokine on the surface of the polymeric film. In some embodiments, the cytokine is IL-2, IL-7, or IL-15, or a combination thereof. In some embodiments, the ratio of the antigen to the cytokine on the surface of the polymeric film is 1:20 to 20:1, including any ratio within this range such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 2:1, 2:3, 2:5, 2:7, 2:9, 2:11, 2:13, 1:15, 2:17, 2:19, 3:1, 3:2, 3:4, 3:5, 3:7, 3:8, 3:10, 3:11, 3:12, 3:13, 3:14, 3:16, 3:17, 3:19, 4:1, 4:3, 4:5, 4:7, 4:9, 4:11, 4:13, 4:15, 4:17, 4:19, 5:1, 5:2, 5:3, 5:4, 5:6, 5:7, 5:8, 5:9, 5:11, 5:12, 5:13, 5:14, 5:16, 5:17, 5:18, 5:19, 6:1, 6:5, 6:7, 6:11, 6:13, 6:17, 6:19, 7:1, 7:2, 7:3, 7:4, 7:5, 7:6, 7:8, 7:9, 7:10, 7:11, 7:12, 7:13, 7:15, 7:16, 7:17, 7:18, 7:19, 7:20, 8:1, 8:3, 8:5, 8:7, 8:9, 8:11, 8:13, 8:15, 8:17, 8:19, 9:1, 9:2, 9:4, 9:5, 9:7, 9:8, 9:10, 9:11, 9:13, 9:14, 9:15, 9:16, 9:17, 9:19, 9:20, 10:1, 10:3, 10:7, 10:9, 10:11, 10:13, 10:17, 10:19, 11:1, 11:2, 11:3, 11:4, 11:5, 11:6, 11:7, 11:8, 11:9, 11:10, 11:12, 11:13, 11:14, 11:15, 11:16, 11:17, 11:18, 11:19, 11:20, 12:1, 12:5, 12:7, 12:11, 12:13, 12:17, 12:19, 13:1, 13:2, 13:3, 13:4, 13:5, 13:6, 13:7, 13:8, 13:9, 13:10, 13:11, 13:12, 13:14, 13:15, 13:16, 13:17, 13:18, 13:19, 13:20, 14:1, 14:3, 14:5, 14:7, 14:9, 14:11, 14:13, 14:15, 14:17, 14:19, 15:1, 15:2, 15:4, 15:6, 15:7, 15:8, 15:10, 15:11, 15:12, 15:13, 15:14, 15:16, 15:17, 15:18, 15:19, 16:1, 16:3, 16:5, 16:7, 16:9, 16:11, 16:13, 16:15, 16:17, 16:19, 17:1, 17:2, 17:3, 17:4, 17:5, 17:6, 17:7, 17:8, 17:9, 17:10, 17:11, 17:12, 17:13, 17:14, 17:15, 17:16, 17:17, 17:18, 17:19, 17:20, 18:1, 18:5, 18:7, 18:11, 18:13, 18:17, 18:19, 19:1, 19:2, 19:3. 19:4, 19:5, 19:6, 19:7, 19:8, 19:9, 19:10, 19:11, 19:12, 19:13, 19:14, 19:15, 19:16, 19:17, 19:18, 19:20, or 20:1.

In certain embodiments, a binding member of a binding-triggered transcriptional switch (BTSS) is attached to the film. A BTSS is a synthetic modular polypeptide or system of interacting polypeptides having an extracellular domain that includes a second member of a specific binding pair (e.g., expressed by a CAR-T cell) that binds a first member of the specific binding pair (e.g., presented on a film), a binding-transducer and an intracellular domain. Upon binding of the first member of the specific binding pair to the BTTS the binding signal is transduced to the intracellular domain such that the intracellular domain becomes activated and performs a function, e.g., transcription activation, within the cell (e.g., CAR-T cell) that it does not perform in the absence of the binding signal.

Examples of BTSS include the synNotch system, the MESA system, the TANGO system, the A2 Notch system, etc. The synNotch receptor may be for example as described in U.S. Pat. No. 9,670,281 and described in more detail below. The MESA system may be as described in WO 2018/081039 A1 and comprises a self-containing sensing and signal transduction system, such that binding of a ligand (first member of the specific binding pair) to the receptor (second member of the specific binding pair) induces signaling to regulate expression of a target gene. In the MESA system, binding of the ligand to the receptor induces dimerization that results in proteolytic trans-cleavage of the system to release a transcriptional activator previously sequestered at the plasma membrane. The TANGO system may be as described in Barnea et al., 2008 Proc. Natl. Acad. Sci. U.S.A., 105 (1): 64-9. Briefly, the TANGO system sequesters a transcription factor to the cell membrane by physically linking it to a membrane-bound receptor (e.g., GPCRs, receptor kinases, Notch, steroid hormone receptors, etc.). Activation of the receptor fusion results in the recruitment of a signaling protein fused to a protease that then cleaves and releases the transcription factor to activate genes in the cell. The A2 Notch system may be as described in WO 2019099689 A1. Briefly, the A2 Notch system incorporates a force sensor cleavage domain which, upon cleavage induced upon binding of a ligand to the receptor, releases the intracellular domain into the cell. In certain embodiments, the second binding member may be present on the surface of a genetically engineered cell, such as, a CAR-T cell expressing a BTTS and a CAR under the control of the BTTS.

In certain cases, the first binding member may bind to a synNotch receptor as described in U.S. Pat. No. 9,670,281. For example, the synNotch receptor may include an extracellular domain that includes the second binding member, where the second binding member is a single-chain Fv (scFv) or a nanobody and the first binding member present on the surface of the film is an antigen to which the single-chain Fv (scFv) or a nanobody binds. In certain cases, the second binding member may be an anti-CD19, anti-mesothelin, anti-GFP antibody, scFv, or a nanobody and the first binding member may be CD19, mesothelin, GFP, respectively.

In certain embodiments, the BTTS is a chimeric Notch polypeptide comprising, from N-terminus to C-terminus and in covalent linkage: a) an extracellular domain comprising the second member of the specific-binding pair that is not naturally present in a Notch receptor polypeptide and that specifically binds to the first member of the specific-binding pair; b) a Notch regulatory region comprising a Lin 12-Notch repeat, an S2 proteolytic cleavage site, and a transmembrane domain comprising an S3 proteolytic cleavage site; c) an intracellular domain comprising a transcriptional activator or a transcriptional repressor that is heterologous to the Notch regulatory region and replaces a naturally-occurring intracellular Notch domain, wherein binding of the first member of the specific-binding pair to the second member of the specific-binding pair induces cleavage at the S2 and S3 proteolytic cleavage sites, thereby releasing the intracellular domain; and a transcriptional control element, responsive to the transcriptional activator, operably linked to a nucleotide sequence encoding a chimeric antigen receptor (CAR). In certain cases, the cell may be a T-cell, such as, those described in U.S. Pat. No. 9,670,281, which is herein incorporated by reference.

In certain embodiments, a co-stimulatory receptor agonist that can be used to modulate an immune cell locally is attached to the surface of a film. For example, the co-stimulatory receptor agonist may be a binding member, e.g., an antibody, that binds CD28, CD137, OX40, GITR, ICOS, CD27, CD30, and HVEM. In certain embodiments, a cytokine is attached to the surface of a film, e.g., a cytokine selected from the group consisting of IL-2, IL-15, IL-12, and GM-CSF. In certain embodiments, a biomolecule is a binding member that binds a checkpoint inhibitor is attached to the surface of a film, e.g. an antibody that binds a checkpoint inhibitor such as one selected from the group consisting of PD-1, PD-L1, CTLA, B7-H1, IDO, TGF-β, BTLA, VISTA, LAG-3, B7-H4, Arginase, MICA, MICB, and TIM-3. In certain embodiments, an adjuvant is attached to the surface of a film, such as CpG, TLR agonist or a STING agonist.

The sequences of the nucleic acids may be selected such that only complementary sequences hybridize and non-complementary sequences do not substantially hybridize. For example, only sequences that have a complementarity of at least 95% or more hybridize. Thus, the sequence of the first single stranded nucleic acid is substantially different from that of the third single stranded nucleic acid such that the second single stranded nucleic acid hybridizes only to the first single stranded nucleic acid and the fourth single stranded nucleic acid specifically hybridizes to the third single stranded nucleic acid.

The present disclosure provides methods for using the antigen-presenting films disclosed herein. The biomolecule-coated films may be used in a variety of in vitro, ex vivo, and in vivo methods.

One or more rounds of antigen-stimulation with a CAR-antigen presenting film can be used to expand numbers of CAR-T cells in culture. In certain embodiments, a method of culture expansion of a CAR-T cell is provided, the method comprising: (a) culturing the CAR-T cell under suitable conditions for growth of the CAR-T cell; (b) contacting the CAR-T cell with a synthetic CAR-antigen presenting film to produce an antigen-stimulated CAR-T cell, wherein the synthetic CAR-antigen presenting film comprises: i) a polymeric film comprising one or more pores; ii) a nucleic acid-polymer conjugate comprising a first single stranded nucleic acid covalently attached to a polymer, wherein the polymer is non-covalently associated with the polymeric film thereby presenting the first single stranded nucleic acid on a surface of the polymeric film; and iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the polymeric film, wherein the antigen specifically binds to the CAR expressed on the CAR-T cell, wherein binding of the antigen to the CAR induces proliferation of the CAR-T cell; (c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and (d) repeating steps (b) and (c).

In certain embodiments, the antigen-presenting films are used in a method of activating and enhancing proliferation of a CAR-T cell. The CAR-T cell may be an effector T cell or a regulatory T cell. The method may be carried out in vitro, in vivo, or ex vivo, for example as further described herein in the context of methods involving CAPP.

In certain embodiments, the polymeric film further comprises a second nucleic acid-polymer conjugate comprising a third single stranded nucleic acid covalently attached to the polymer, wherein the polymer is non-covalently associated with the polymeric film thereby presenting the third single stranded nucleic acid on the surface of the polymeric film and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric film.

In certain embodiments, the polymeric film further comprises a third nucleic acid-polymer conjugate comprising a fifth single stranded nucleic acid covalently attached to the polymer, wherein the polymer is non-covalently associated with the polymeric film thereby presenting the fifth single stranded nucleic acid on the surface of the polymeric film and a cytokine-nucleic acid conjugate comprising a sixth single stranded nucleic acid covalently attached to a cytokine, wherein the sixth single stranded nucleic acid is complementary to the fifth single stranded nucleic acid and is associated with the fifth single stranded nucleic acid via hybridization thereby presenting the cytokine on the surface of the polymeric film. In some embodiments, the cytokine is IL-2, IL-7, or IL-15, or a combination thereof.

The present disclosure provides methods for making the antigen-presenting films disclosed herein. In certain embodiments, the method comprises covalently or non-covalently linking a nucleic acid to a first polymer to generate a nucleic acid-polymer conjugate, wherein the nucleic acid is a first single stranded nucleic acid; mixing a solution comprising the nucleic acid-polymer conjugate and a second polymer in a solvent; film casting the solution to generate a polymeric film comprising the second polymer, wherein the polymer region of the nucleic acid-polymer conjugate is non-covalently associated with the polymeric film thereby presenting the first single stranded nucleic acid on a surface of the polymeric film; attaching to the polymeric film a second single stranded nucleic acid having a sequence complementary to the first single stranded nucleic acid by hybridization; and covalently or non-covalently attaching the second single stranded nucleic acid to an antigen to generate the antigen-presenting film.

In certain embodiments, the method comprises covalently attaching the second single stranded nucleic acid to the antigen prior to attaching the second single stranded nucleic acid to the polymeric film.

In certain embodiments, the method comprises covalently attaching the second single stranded nucleic acid to the antigen after attaching the second single stranded nucleic acid to the polymeric film.

In certain embodiments, the method comprises generating a plurality of nucleic acid-polymer conjugates, where the plurality of nucleic acid-polymer conjugates at least include a first nucleic acid-polymer conjugate comprising a first single stranded nucleic acid covalently linked to a first polymer molecule; and a second nucleic acid-polymer conjugate comprising a third single stranded nucleic acid covalently linked to a first polymer molecule, where the first single stranded nucleic acid and the third single stranded nucleic acid have different sequences. In certain cases, the plurality of nucleic acid-polymer conjugates includes at least three, at least four, at least five, or more different nucleic acid-polymer conjugates each comprising a different single stranded nucleic acid, where the sequences are sufficiently different to enable hybridization to different nucleic acids, which are in turn, attached to different molecules, e.g., to allow functionalization of the surface of the film with antigens, antibodies, costimulatory ligands, cytokines, and/or other immunomodulatory molecules, a binding member of a BTTS, a self-peptide, and/or other molecules.

As noted herein, the plurality of nucleic acid-polymer conjugates can be included in a desired ratio. For example, the first nucleic acid-polymer conjugate and the second nucleic acid-polymer conjugate may be included in the solution at a ratio of 100:1. 50:1, 10:1, 5:1, 3:1, 2:1, 1:1, 1:2, 1:3, 1:5, 1:10, 1:50, or 1:100. The step of covalently linking a nucleic acid to a first polymer to generate a nucleic acid-polymer conjugate may be carried out using standard chemistry suitable for the reactive groups being used for the covalent attachment. Examples of suitable reactive groups include thiol-maleimide, amino and carboxyl groups, thiol and carboxyl groups, and the like.

The step of mixing the solution comprising the nucleic acid-polymer conjugate and second polymer can be carried out in any solvent suitable for film casting. Examples of suitable common solvents that may be used include, but are not limited to, dimethyl oxalate (DMO), ethylene carbonate (EC), N-methyl acetamide (NMA), dimethyl sulfoxide (DMSO), acetic acid (AA), 1,4-dioxane (DO), dimethyl carbonate (DMC), chloroform, dichloromethane (DCM), naphthalene, sulfalene, trimethylurea, ethylene glycol or other glycols and polyglycols, N-methyl pyrrolidone (NMP), ethylene carbonate, hexane, cyclohexane, trifluoroethanol (TFE), ethanol, acetic acid, and water, and combinations thereof. In certain embodiments, the solvent is a combination of TFE and water.

### Kits

Kits are provided that include a CAR-antigen presenting particle or film disclosed herein. The kit may further comprise a CAR-T cell that specifically binds to the antigen presented on the CAR-antigen presenting particle or film. In addition, a kit may further comprise media suitable for culturing CAR-T cells. In some embodiments, the kit further comprises a vector for genetically modifying a T cell to produce a chimeric antigen receptor. In some embodiments, the kits comprises nucleic acid(s) or viral particle(s) encoding the CAR, where the CAR, once expressed on a T cell, specifically binds to the antigen presented on the CAR-antigen presenting particle. The nucleic acid(s) encoding the CAR may be part of a vector, e.g., a viral vector such as a lentiviral vector. The viral particle(s) encoding the CAR may be a lentiviral particle, e.g., a harvested lentiviral particle. Additionally, the kit may include transfection agents, buffers, media, cytokines (e.g., IL-2, IL-7, and/or IL-15), tissue culture plates, flasks, test tubes, vials, and the like.

The kit may also provide a delivery device for administration of CAR-T cells to a patient. For example, kits may comprise a container having a sterile access port (e.g., the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-158 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A method of culture expansion of a chimeric antigen receptor (CAR)-T cell, the method comprising:
(a) culturing the CAR-T cell under suitable conditions for growth of the CAR-T cell;
(b) contacting the CAR-T cell with a synthetic CAR-antigen presenting particle to produce an antigen-stimulated CAR-T cell, wherein the synthetic CAR-antigen presenting particle comprises:
   i) a polymeric core;
   ii) a nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; and
   iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR induces proliferation of the CAR-T cell;
(c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and
(d) repeating steps (b) and (c).

2. The method of aspect 1, wherein step (b) is repeated about every 5-15 days.

3. The method of aspect 1 or 2, wherein step (d) is repeated at least 1 time.

4. The method of aspect 3, wherein step (d) is repeated at least 2-10 times.

5. The method of any one of aspects 1-4, wherein the polymeric core comprises poly(D,L-lactide-co-glycolide) (PLGA), poly(lactic acid) (PLA), polyglycolic acid (PGA), poly(e-caprolactone) (PCL), or polyethylene glycol (PEG), or a combination thereof.

6. The method of any one of aspects 1-5, wherein the polymer of the nucleic acid-polymer conjugate comprises a poly(D,L-lactide-co-glycolide) (PLGA)-polyethylene glycol (PEG) block polymer (PLGA-block-PEG) or a poly(D,L-lactide) (PLA)-polyethylene glycol (PEG) block polymer (PLA-block-PEG) or a poly(e-caprolactone) (PCL)-polyethylene glycol (PEG) block polymer (PCL-block-PEG).

7. The method of any one of aspects 1-6, wherein the first single stranded nucleic acid comprises deoxyribonucleic acid (DNA) or ribonucleic acid (RNA) or peptide nucleic acid (PNA).

8. The method of aspect 7, wherein the DNA or RNA or PNA comprises 5-200 bases.

9. The method of any one of aspects 1-8, wherein the second single stranded nucleic acid comprises deoxyribonucleic acid (DNA) or ribonucleic acid (RNA) or peptide nucleic acid (PNA), optionally wherein the DNA or RNA or PNA comprises 5-200 bases.

10. The method of any one of aspects 1-9, wherein the first single stranded nucleic acid comprises at least 4 contiguous bases complementary to at least 4 contiguous bases in the second single stranded nucleic acid.

11. The method of any one of aspects 1-10, wherein the particle is a nanoparticle having a diameter ranging from 50 nm-500 nm.

12. The method of any one of aspects 1-11, wherein the particle is a microparticle having a diameter ranging from 0.5 μm-50 μm.

13. The method of any one of aspects 1-12, wherein the CAR comprises a transmembrane domain linked to an extracellular antigen binding domain and an intracellular signaling domain, wherein the extracellular antigen-binding domain specifically binds to the antigen.

14. The method of aspect 13, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the antigen.

15. The method of aspect 13 or 14, wherein the intracellular signaling domain is a CD3-zeta intracellular signaling domain or a ZAP-70 intracellular signaling domain.

16. The method of aspect 13 or 14, wherein the intracellular signaling domain comprises an immunoreceptor tyrosine-based activation motif (ITAM).

17. The method of any one of aspects 13-16, wherein the CAR further comprises a costimulatory domain.

18. The method of aspect 17, wherein the costimulatory domain is a 4-1BB, CD28, ICOS, OX-40, BTLA, CD27, CD30, GITR, or HVEM costimulatory domain.

19. The method of any one of aspects 13-18, wherein the transmembrane domain is a CD8, CD28, Megf10, FcRγ, Bai1, MerTK, TIM4, Stabilin-1, Stabilin-2, RAGE, CD300f, integrin subunit av, integrin subunit β5, CD36, LRP1, SCARF1, C1Qa, Axl, CD45, or CD86 transmembrane domain.

20. The method of any one of aspects 1-19, wherein the antigen is selected from the group consisting of: CD19, HER2, epidermal growth factor receptor (EGFR), green fluorescent protein (GFP), fluorescein isothiocyanate (FITC), CD19, CD20, CD22, CD38, CD30, CA125, MUC-1, prostate-specific membrane antigen (PSMA), CD44 surface adhesion molecule, mesothelin, carcinoembryonic antigen (CEA), EGFRvIII, vascular endothelial growth factor receptor-2 (VEGFR2), high molecular weight-melanoma associated antigen (HMW-MAA), MAGE-A1, IL-13R-a2, GD2, MET, GPC3, CD70, EphA2, EpCAM, CLDN18, BCMA, CA9, and fibroblast activation protein (FAP).

21. The method of aspect 20, wherein the antigen is EGFR, the antigen-binding domain comprises an anti-EGFR nanobody, the transmembrane domain is a CD8 transmembrane domain or CD28 transmembrane domain, the intracellular signaling domain is a CD3-zeta intracellular signaling domain, and the costimulatory domain is a 4-1BB or CD28 costimulatory domain.

22. The method of any one of aspects 1-21, wherein the CAR-antigen presenting particle further comprises a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

23. The method of aspect 22, wherein the costimulatory ligand is an anti-CD28 antibody, CD80, CD86, an anti-41BB antibody, an anti-ICOS antibody, or an anti-OX40 antibody.

24. The method of aspect 23, wherein the ratio of the antigen to the costimulatory ligand on the surface of the CAR-antigen presenting particle is 1:20 to 20:1.

25. The method of any one of aspects 1-24, wherein the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

26. The method of aspect 25, wherein the cytokine is IL-2, IL-7, or IL-15.

27. The method of aspect 25 or 26, wherein the antigen and the cytokine are present at a ratio of 10:1 to 1:10.

28. The method of any one of aspects 1-27, further comprising contacting the CAR-T cell with a synthetic cytokine-presenting particle, wherein the synthetic cytokine-presenting particle comprises a cytokine presented on a surface of a synthetic particle.

29. The method of aspect 28, wherein the synthetic particle is a polymeric particle, a magnetic bead, a gold particle, or a liposome.

30. The method of aspect 28 or 29, wherein the cytokine is IL-2, IL-7, or IL-15.

31. The method of any one of aspects 1-30, further comprising administering a therapeutically effective amount of the antigen-stimulated CAR-T cell to a subject in need thereof after said culture expansion.

32. The method of aspect 31, wherein the CAR-T cell is administered locally into a tumor or intravenously.

33. The method of aspect 31 or 32, further comprising administering a therapeutically effective amount of the CAR-antigen presenting particle in combination with the antigen-stimulated CAR-T cell to a subject in need thereof after said culture expansion.

34. The method of any one of aspects 31-33, wherein the subject has an impaired T cell response to the antigen.

35. The method of any one of aspects 31-34, wherein the subject is immunocompromised because of treatment with chemotherapeutic agents or immunosuppressive agents.

36. The method of any one of aspects 1-35, wherein the CAR-T cell is an effector T cell that has been genetically modified to express the CAR, or wherein the CAR-T cell is a regulatory T cell (Treg) that has been genetically modified to express the CAR.

37. The method of aspect 36, wherein the effector T cell is a helper CD4⁺ T cell, a cytotoxic CD8⁺ T cell, a natural killer T cell, or a gamma delta T cell.

38. The method of aspect 36 or 37, wherein the effector T cell or Treg is autologous or allogeneic.

39. The method of any one of aspects 1-38, wherein the antigen-stimulated CAR-T cell binds specifically to a cancer antigen, a B-cell antigen for autoreactive B cell removal, or a fibrosis antigen.

40. The method of aspect 39, wherein the subject has a cancer expressing the cancer antigen, and the antigen-stimulated CAR-T cell has anti-cancer activity.

41. The method of aspect 39 or 40, wherein the cancer is leukemia, lymphoma, myeloma, prostate cancer, breast cancer, lung cancer, kidney cancer, lung cancer, ovarian cancer, intestine cancer, or glioblastoma.

42. The method of aspect 39, wherein the subject has fibrotic tissue expressing the fibrosis antigen, and the antigen-stimulated CAR-T cell has anti-fibrosis activity.

43. The method of aspect 42, wherein the fibrosis antigen is fibroblast activation protein (FAP).

44. The method of any one of aspects 1-43, wherein the particle is superparamagnetic, wherein the method further comprises isolating the particle by using a magnetic separation technique.

45. The method of any one of aspects 1-44, wherein expression of the CAR in the CAR-T cell is inducible.

46. The method of aspect 45, wherein the CAR-T cell comprises a binding-triggered transcriptional switch that controls expression of the CAR by the CAR-T cell.

47. The method of aspect 46, wherein the binding-triggered transcriptional switch comprises a chimeric Notch polypeptide.

48. The method of any one of aspects 1-47, wherein the CAR-T cell comprises an insertion of a recombinant polynucleotide comprising a coding sequence encoding the CAR at a TRAC locus of the CAR-T cell genome.

49. The method of any one of aspects 1-48, further comprising enriching for memory progenitor CAR-T cells generated from the antigen-stimulated CAR-T cell.

50. The method of any one of aspects 1-49, wherein step (b) is performed with a ratio of the synthetic CAR-antigen presenting particle to the CAR-T cell in a range of 2:1 to 10:1.

51. The method of any one of aspects 1-50, wherein step (b) is performed with a ratio of the synthetic CAR-antigen presenting particle to the CAR-T cell of at least 5:1 or at least 10:1.

52. A method of activating a chimeric antigen receptor (CAR)-T cell, the method comprising contacting the CAR-T cell with a synthetic CAR-antigen presenting particle to produce an activated CAR-T cell, wherein the synthetic CAR-antigen presenting particle comprises:
i) a polymeric core;
ii) a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core;
iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR activates the CAR-T cell; and
iv) a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the a costimulatory ligand on the surface of the polymeric particle.

53. The method of aspect 52, wherein the method is performed in vitro, ex vivo, or in vivo.

54. The method of aspect 52 or 53, wherein the costimulatory ligand is an anti-CD28 antibody, CD80, CD86, an anti-41BB antibody, an anti-ICOS antibody, or an anti-OX40 antibody.

55. The method of any one of aspects 52-54, wherein the ratio of the antigen to the costimulatory ligand on the surface of the CAR-antigen presenting particle is 1:20 to 20:1.

56. The method of any one of aspects 52-55, wherein said contacting is performed in presence of interleukin-2 (IL-2) or a combination of interleukin-7 (IL-7) and interleukin-15 (IL-15).

57. The method of any one of aspects 52-56, wherein the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

58. The method of aspect 57, wherein the cytokine is IL-2, IL-7, or IL-15.

59. The method of aspect 57 or 58, wherein the antigen and the cytokine are present at a ratio of 10:1 to 1:10.

60. The method of any one of aspects 52-59, further comprising contacting the CAR-T cell with a synthetic cytokine-presenting particle, wherein the synthetic cytokine-presenting particle comprises a cytokine presented on a surface of a synthetic particle.

61. The method of aspect 60, wherein the synthetic particle is a polymeric particle, a magnetic bead, a gold particle, or a liposome.

62. The method of aspect 60 or 61, wherein the cytokine is IL-2, IL-7, or IL-15.

63. A method of culture expansion of a chimeric antigen receptor (CAR)-T cell, the method comprising:
(a) culturing the CAR-T cell under suitable conditions for growth of the CAR-T cell;
(b) contacting the CAR-T cell with a synthetic CAR-antigen presenting film to produce an antigen-stimulated CAR-T cell, wherein the synthetic CAR-antigen presenting film comprises:
   i) a polymeric film comprising one or more pores;
   ii) a nucleic acid-polymer conjugate comprising a first single stranded nucleic acid covalently attached to a polymer, wherein the polymer is non-covalently associated with the polymeric film thereby presenting the first single stranded nucleic acid on a surface of the polymeric film; and
   iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the polymeric film, wherein the antigen specifically binds to the CAR expressed on the CAR-T cell, wherein binding of the antigen to the CAR induces proliferation of the CAR-T cell;
(c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and
(d) repeating steps (b) and (c).

64. The method of aspect 63, wherein the polymeric film comprises pores having a diameter of between 1 μm to 5 μm, optionally wherein the polymeric film comprises pores having a diameter of between 1 μm to 2 μm.

65. The method of aspect 63 or 64, wherein the polymeric film comprises a thickness of between 1 μm and 100 μm.

66. The method of any one of aspects 63-65, wherein step (b) is repeated about every 5-15 days.

67. The method of any one of aspects 63-66, wherein step (d) is repeated at least 1 time.

68. The method of aspect 67, wherein step (d) is repeated at least 2-10 times.

69. The method of any one of aspects 63-68, wherein the polymeric film comprises poly(D,L-lactide-co-glycolide) (PLGA), poly(lactic acid) (PLA), polyglycolic acid (PGA), poly(e-caprolactone) (PCL), or polyethylene glycol (PEG).

70. The method of any one of aspects 63-69, wherein the polymeric film comprises polycaprolactone (PCL) and polyethylene glycol (PEG).

71. The method of any one of aspects 63-70, wherein the polymer of the nucleic acid-polymer conjugate comprises a poly(D,L-lactide-co-glycolide) (PLGA)-polyethylene glycol (PEG) block polymer (PLGA-block-PEG) or a poly(D,L-lactide) (PLA)-polyethylene glycol (PEG) block polymer (PLA-block-PEG) or a poly(e-caprolactone) (PCL)-polyethylene glycol (PEG) block polymer (PCL-block-PEG).

72. The method of any one of aspects 63-71, wherein the first single stranded nucleic acid comprises deoxyribonucleic acid (DNA) or ribonucleic acid (RNA) or peptide nucleic acid (PNA).

73. The method of aspect 72, wherein the DNA or RNA or PNA comprises 5-200 bases.

74. The method of any one of aspects 63-73, wherein the second single stranded nucleic acid comprises deoxyribonucleic acid (DNA) or ribonucleic acid (RNA) or peptide nucleic acid (PNA), optionally wherein the DNA or RNA or PNA comprises 5-200 bases.

75. The method of any one of aspects 63-74, wherein the first single stranded nucleic acid comprises at least 4 contiguous bases complementary to at least 4 contiguous bases in the second single stranded nucleic acid.

76. The method of any one of aspects 63-75, wherein the CAR comprises a transmembrane domain linked to an extracellular antigen binding domain and an intracellular signaling domain, wherein the extracellular antigen-binding domain specifically binds to the antigen.

77. The method of aspect 76, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the antigen.

78. The method of aspect 76 or 77, wherein the intracellular signaling domain is a CD3-zeta intracellular signaling domain or a ZAP-70 intracellular signaling domain.

79. The method of aspect 76 or 77, wherein the intracellular signaling domain comprises an immunoreceptor tyrosine-based activation motif (ITAM).

80. The method of any one of aspects 76-79, wherein the CAR further comprises a costimulatory domain.

81. The method of aspect 80, wherein the costimulatory domain is a 4-1BB, CD28, ICOS, OX-40, BTLA, CD27, CD30, GITR, or HVEM costimulatory domain.

82. The method of any one of aspects 76-81, wherein the transmembrane domain is a CD8, CD28, Megf10, FcRγ, Bai1, MerTK, TIM4, Stabilin-1, Stabilin-2, RAGE, CD300f, integrin subunit av, integrin subunit β5, CD36, LRP1, SCARF1, C1Qa, Axl, CD45, or CD86 transmembrane domain.

83. The method of any one of aspects 63-82, wherein the antigen is selected from the group consisting of: CD19, HER2, epidermal growth factor receptor (EGFR), green fluorescent protein (GFP), fluorescein isothiocyanate (FITC), CD19, CD20, CD22, CD38, CD30, CA125, MUC-1, prostate-specific membrane antigen (PSMA), CD44 surface adhesion molecule, mesothelin, carcinoembryonic antigen (CEA), EGFRvIII, vascular endothelial growth factor receptor-2 (VEGFR2), high molecular weight-melanoma associated antigen (HMW-MAA), MAGE-A1, IL-13R-a2, GD2, MET, GPC3, CD70, EphA2, EpCAM, CLDN18, BCMA, CA9, and fibroblast activation protein (FAP).

84. The method of aspect 83, wherein the antigen is EGFR, the antigen-binding domain comprises an anti-EGFR nanobody, the transmembrane domain is a CD8 transmembrane domain or a CD28 transmembrane domain, the intracellular signaling domain is a CD3-zeta intracellular signaling domain, and the costimulatory domain is a 4-1BB or CD28 costimulatory domain.

85. The method of any one of aspects 63-84, wherein the polymeric film further comprises a second nucleic acid-polymer conjugate comprising a third single stranded nucleic acid covalently attached to the polymer, wherein the polymer is non-covalently associated with the polymeric film thereby presenting the third single stranded nucleic acid on the surface of the polymeric film and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric film.

86. The method of aspect 85, wherein the costimulatory ligand is an anti-CD28 antibody, CD80, CD86, an anti-41BB antibody, an anti-ICOS antibody, or an anti-OX40 antibody.

87. The method of aspect 85 or 86, wherein the ratio of the antigen to the costimulatory ligand on the surface of the polymeric film is 1:20 to 20:1.

88. The method of any one of aspects 63-87, wherein the polymeric film further comprises a third nucleic acid-polymer conjugate comprising a fifth single stranded nucleic acid covalently attached to the polymer, wherein the polymer is non-covalently associated with the polymeric film thereby presenting the fifth single stranded nucleic acid on the surface of the polymeric film and a cytokine-nucleic acid conjugate comprising a sixth single stranded nucleic acid covalently attached to a cytokine, wherein the sixth single stranded nucleic acid is complementary to the fifth single stranded nucleic acid and is associated with the fifth single stranded nucleic acid via hybridization thereby presenting the cytokine on the surface of the polymeric film.

89. The method of aspect 88, wherein the cytokine is IL-2, IL-7, or IL-15.

90. The method of aspect 88 or 89, wherein the antigen and the cytokine are present at a ratio of 10:1 to 1:10.

91. The method of any one of aspects 63-90, further comprising administering a therapeutically effective amount of the antigen-stimulated CAR-T cell to a subject in need thereof after said culture expansion.

92. The method of aspect 91, wherein the CAR-T cell is administered locally into a tumor or fibrotic tissue, or administered intravenously.

93. The method of any one of aspects 63-92, wherein the CAR-T cell is an effector T cell that has been genetically modified to express the CAR, or wherein the CAR-T cell is a regulatory T cell (Treg) that has been genetically modified to express the CAR.

94. The method of aspect 93, wherein the effector T cell is a helper CD4+ T cell, a cytotoxic CD8+ T cell, a natural killer T cell, or a gamma delta T cell.

95. The method of aspect 93 or 94, wherein the effector T cell or Treg is autologous or allogeneic.

96. The method of any one of aspects 63-95, wherein the CAR-T cell comprises a binding-triggered transcriptional switch that controls expression of the CAR by the CAR-T cell.

97. The method of aspect 96, wherein the binding-triggered transcriptional switch comprises a chimeric Notch polypeptide.

98. The method of any one of aspects 63-97, wherein the antigen-stimulated CAR-T cell binds specifically to a cancer antigen, a B-cell antigen for autoreactive B cell removal, or a fibrosis antigen.

99. The method of aspect 98, wherein the subject has a cancer expressing the cancer antigen, and the antigen-stimulated CAR-T cell has anti-cancer activity.

100. The method of aspect 98 or 99, wherein the cancer is leukemia, lymphoma, myeloma, prostate cancer, breast cancer, lung cancer, kidney cancer, lung cancer, ovarian cancer, intestine cancer, or glioblastoma.

101. The method of aspect 98, wherein the subject has fibrotic tissue expressing the fibrosis antigen, and the antigen-stimulated CAR-T cell has anti-fibrosis activity.

102. The method of aspect 101, wherein the fibrosis antigen is fibroblast activation protein (FAP).

103. A composition comprising a synthetic CAR-antigen presenting particle (CAPP), the CAPP comprising:
(a) a polymeric core;
(b) a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core;
(c) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to a CAR expressed on a CAR-T cell, wherein binding of the antigen to the CAR activates the CAR-T cell; and
(d) a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

104. The composition of aspect 103, wherein the costimulatory ligand is an anti-CD28 antibody, CD80, CD86, an anti-41BB antibody, an anti-ICOS antibody, or an anti-OX40 antibody.

105. The composition of aspect 103 or 104, wherein the ratio of the antigen to the costimulatory ligand on the surface of the CAR-antigen presenting particle is 1:20 to 20:1.

106. The composition of any one of aspects 103-105, wherein the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

107. The composition of aspect 106, wherein the cytokine is IL-2, IL-7, or IL-15.

108. The composition of aspect 106 or 107, wherein the antigen and the cytokine are present at a ratio of 10:1 to 1:10.

109. The composition of any one of aspects 103-108, further comprising interleukin-2 (IL-2).

110. The composition of aspect 109, further comprising interleukin-7 (IL-7) and/or interleukin-15 (IL-15).

111. The composition of any one of aspects 103-110, further comprising the CAR-T cell, wherein the antigen specifically binds to the CAR expressed on the CAR-T cell.

112. The composition of any one of aspects 103-111, further comprising a pharmaceutically acceptable excipient.

113. A composition comprising a synthetic CAR-antigen presenting particle (CAPP) for use in a method of immunotherapy, the CAPP comprising:
(a) a polymeric core;
(b) a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core;
(c) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to a CAR expressed on a CAR-T cell, wherein binding of the antigen to the CAR activates the CAR-T cell; and
(d) a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

114. The composition of aspect 113, wherein the costimulatory ligand is an anti-CD28 antibody, CD80, CD86, an anti-41BB antibody, an anti-ICOS antibody, or an anti-OX40 antibody.

115. The composition of aspect 113 or 114, wherein the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

116. The composition of aspect 115, wherein the cytokine is IL-2, IL-7, or IL-15.

117. A kit comprising the composition of any one of aspects 113-116 and instructions for using the CAPP.

118. A method of culture expansion of a chimeric antigen receptor (CAR)-T cell, the method comprising:
(a) culturing the CAR-T cell under suitable conditions for growth of the CAR-T cell;
(b) contacting the CAR-T cell with a CAR-antigen presenting particle to produce an antigen-stimulated CAR-T cell, wherein the CAR-antigen presenting particle comprises an antigen and a binding agent that selectively binds to a co-stimulatory molecule presented on a surface of a synthetic particle, wherein binding of the antigen to the CAR induces proliferation of the CAR-T cell;
(c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and
(d) repeating steps (b) and (c).

119. The method of aspect 118, wherein the costimulatory ligand is an anti-CD28 antibody, CD80, CD86, an anti-41BB antibody, an anti-ICOS antibody, or an anti-OX40 antibody.

120. The method of aspect 118 or 119, wherein the ratio of the antigen to the costimulatory ligand on the surface of the synthetic particle is 1:20 to 20:1.

121. The method of any one of aspects 118-120, wherein the synthetic particle further comprises a cytokine presented on the surface of the synthetic particle.

122. The method of aspect 121, wherein the cytokine is IL-2, IL-7, or IL-15.

123. The method of aspect 121 or 122, wherein the antigen and the cytokine are present at a ratio of 10:1 to 1:10.

124. The method of any one of aspects 118-123, wherein the synthetic particle is a polymeric particle, a magnetic bead, a gold particle, or a liposome.

125. The method of aspect 124, further comprising isolating the CAR-antigen presenting particle comprising the magnetic bead by using a magnetic separation technique after said culture expansion of the chimeric antigen receptor (CAR)-T cell.

126. The method of any one of aspects 118-125, wherein step (b) is repeated about every 5-15 days.

127. The method of any one of aspects 118-126, wherein step (d) is repeated at least 1 time.

128. The method of aspect 127, wherein step (d) is repeated at least 2-10 times.

129. The method of any one of aspects 118-128, wherein the synthetic particle is a nanoparticle having a diameter ranging from 50 nm-500 nm.

130. The method of any one of aspects 118-129, wherein the synthetic particle is a microparticle having a diameter ranging from 0.5 μm-50 μm.

131. The method of any one of aspects 118-130, wherein the CAR comprises a transmembrane domain linked to an extracellular antigen binding domain and an intracellular signaling domain, wherein the extracellular antigen-binding domain specifically binds to the antigen.

132. The method of aspect 131, wherein the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the antigen.

133. The method of aspect 131 or 132, wherein the intracellular signaling domain is a CD3-zeta intracellular signaling domain or a ZAP-70 intracellular signaling domain.

134. The method of aspect 131 or 132, wherein the intracellular signaling domain comprises an immunoreceptor tyrosine-based activation motif (ITAM).

135. The method of any one of aspects 131-134, wherein the CAR further comprises a costimulatory domain.

136. The method of aspect 135, wherein the costimulatory domain is a 4-1BB, CD28, ICOS, OX-40, BTLA, CD27, CD30, GITR, or HVEM costimulatory domain.

137. The method of any one of aspects 131-136, wherein the transmembrane domain is a CD8, CD28, Megf10, FcRγ, Bai1, MerTK, TIM4, Stabilin-1, Stabilin-2, RAGE, CD300f, integrin subunit av, integrin subunit β5, CD36, LRP1, SCARF1, C1Qa, Axl, CD45, or CD86 transmembrane domain.

138. The method of any one of aspects 118-137, wherein the antigen is selected from the group consisting of: CD19, HER2, epidermal growth factor receptor (EGFR), green fluorescent protein (GFP), fluorescein isothiocyanate (FITC), CD19, CD20, CD22, CD38, CD30, CA125, MUC-1, prostate-specific membrane antigen (PSMA), CD44 surface adhesion molecule, mesothelin, carcinoembryonic antigen (CEA), EGFRvIII, vascular endothelial growth factor receptor-2 (VEGFR2), high molecular weight-melanoma associated antigen (HMW-MAA), MAGE-A1, IL-13R-a2, GD2, MET, GPC3, CD70, EphA2, EpCAM, CLDN18, BCMA, CA9, and fibroblast activation protein (FAP).

139. The method of aspect 138, wherein the antigen is EGFR, the antigen-binding domain comprises an anti-EGFR nanobody, the transmembrane domain is a CD8 transmembrane domain or a CD28 transmembrane domain, the intracellular signaling domain is a CD3-zeta intracellular signaling domain, and the costimulatory domain is a 4-1BB or CD28 costimulatory domain.

140. The method of any one of aspects 118-139, further comprising administering a therapeutically effective amount of the antigen-stimulated CAR-T cell to a subject in need thereof after said culture expansion.

141. The method of aspect 140, wherein the CAR-T cell is administered locally into a tumor or fibrotic tissue or administered intravenously.

142. The method of aspect 140 or 141, further comprising administering a therapeutically effective amount of the CAR-antigen presenting particle in combination with the antigen-stimulated CAR-T cell to a subject in need thereof after said culture expansion.

143. The method of any one of aspects 140-142, wherein the subject has an impaired T cell response to the antigen.

144. The method of any one of aspects 140-143, wherein the subject is immunocompromised because of treatment with chemotherapeutic agents or immunosuppressive agents.

145. The method of any one of aspects 118-144, wherein the CAR-T cell is an effector T cell that has been genetically modified to express the CAR, or wherein the CAR-T cell is a regulatory T cell (Treg) that has been genetically modified to express the CAR.

146. The method of aspect 145, wherein the effector T cell is a helper CD4+ T cell, a cytotoxic CD8+ T cell, a natural killer T cell, or a gamma delta T cell.

147. The method of aspect 145 or 146, wherein the effector T cell or Treg is autologous or allogeneic.

148. The method of any one of aspects 118-147, wherein expression of the CAR in the CAR-T cell is inducible.

149. The method of aspect 148, wherein the CAR-T cell comprises a binding-triggered transcriptional switch that controls expression of the CAR by the CAR-T cell.

150. The method of aspect 192, wherein the binding-triggered transcriptional switch comprises a chimeric Notch polypeptide.

151. The method of any one of aspects 118-150, wherein the antigen-stimulated CAR-T cell binds specifically to a cancer antigen, a B-cell antigen for autoreactive B cell removal, or a fibrosis antigen.

152. The method of aspect 151, wherein the subject has a cancer expressing the cancer antigen, and the antigen-stimulated CAR-T cell has anti-cancer activity.

153. The method of aspect 151 or 152, wherein the cancer is leukemia, lymphoma, myeloma, prostate cancer, breast cancer, lung cancer, kidney cancer, lung cancer, ovarian cancer, intestine cancer, or glioblastoma.

154. The method of aspect 151, wherein the subject has fibrotic tissue expressing the fibrosis antigen, and the antigen-stimulated CAR-T cell has anti-fibrosis activity.

155. The method of aspect 154, wherein the fibrosis antigen is fibroblast activation protein (FAP).

156. The method of any one of aspects 118-155, wherein step (b) is performed with a ratio of the synthetic CAR-antigen presenting particle to the CAR-T cell in a range of 2:1 to 10:1.

157. The method of any one of aspects 118-156, wherein step (b) is performed with a ratio of the synthetic CAR-antigen presenting particle to the CAR-T cell of at least 5:1 or at least 10:1.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, dimensions, etc.) but some experimental errors and deviations should be accounted for.

### Materials and Methods

### 1. Synthesis of Thiol-Modified DNA

3′ Thiol-modified DNA with 17 bases are synthesized on 3′ thiol-modifier 6 S—S CPG beads (Glen Research #10-1936-02) using an Expedite 8909 DNA synthesizer. DNA are retrieved from the beads by 70° C. incubation for 20 mins in the presence of AMA solution (Ammonium hydroxide:Methylamine=1:1, v/v), followed by vacuum evaporation (SpeedVac, ThermoFisher #SPD121P-230) for 3 hours to remove AMA. DNA reconstituted in TE buffer (Tris-EDTA, 10 mM, pH7.5) are then filtered through 0.22 μm filter (Millipore #UFC30GV00), and kept in −20° C. for stock.

### 2. Synthesis of Amphiphilic Polymer-DNA

Synthesized DNA are treated with Tris(2-carboxyethyl)phosphine hydrochloride solution (TCEP, Sigma #646547) with 100× molar excess at 37° C. for 1 hour to remove the disulfide protection, and then purified using a size-exclusion chromatography (Glen Research #61-5010). Freshly prepared thiol-DNA are mixed with 200 μM maleimide-functionalized polymers (i.e. Poly(lactide-co-glycolide)-b-poly(ethylene glycol)-maleimide, Mw 10,000:5000 Da, Akina #AI053, PLGA-PEG-MAL) at different ratios in the solvent of dimethylformamide (DMF)/H₂O (90:10, v/v) with the addition of 0.2% triethanolamine, and incubated at room temperature for overnight to complete the reaction. The next day the solvent is removed by vacuum evaporation heated to 70° C. for 3 hours, and the polymer-DNA is stocked in −20° C. The product quality is determined by running TBE-Urea gel electrophoresis (15%, Invitrogen #EC68855).

3. Fabrication of Polymeric Particles with DNA Scaffolds

Polymeric PLGA particles are fabricated using a single emulsion method in the mixture of aqueous buffer and organic solvent. Amphiphilic polymer-DNA is reconstituted in 200 μL solvent mixture (ethyl acetate:H₂O=1:1, v/v), and mixed with certain amount of the mainstream (unmodified) polymer (Poly-lactide-co-glycolide, 50:50, Mw 38,000-54,000, Sigma #719900, or Polylactic acid, Mw 60,000, Sigma #38534) dissolved in ethyl acetate or dichloromethane (DCM) according to the target particle size and degradation profile, with the addition of aqueous buffer (10 mM sodium citrate, 600 mM Na⁺, pH 3.0). For PLGA microparticles with 1-5 μm diameter, 100 nmol PLGA-DNA and 50 mg PLGA (Sigma #719900) are mixed in 0.5 mL ethyl acetate and 0.5 mL aqueous buffer; for PLA microparticles with 1-5 μm diameter, 200 nmol PLGA-DNA and 50 mg PLA (Sigma #38534) are mixed in 0.5 mL DCM and 0.5 mL aqueous buffer; for PLGA particles with around 500 nm diameter, 100 nmol PLGA-DNA and 10 mg PLGA (Sigma #719900) are mixed in 0.5 mL ethyl acetate and 1 mL aqueous buffer; and for PLGA particles with around 200 nm diameter, 100 nmol PLGA-DNA and 10 mg PLGA (Sigma #719900) are mixed in 0.5 mL ethyl acetate and 1 mL aqueous buffer with 1% polyvinyl alcohol (PVA, Sigma #81381) addition. The whole mixture is then vortexed and probe-sonicated on ice at 7-8 W for 5×5 s with 10 s intervals, and immediately added with 9 mL 0.2% PVA and stirred in the hood for 3 hours for ethyl acetate to evaporate. Particles are filtered through 40 μm cell strainer (Sigma #CLS431750), and centrifuged at 10,000×g for 10 mins to collect the pellet, and then resuspended in TE buffer (10 mM Tris-HCl, pH 8.0) with 0.01% Tween-20 for washing. This protocol remains the same for later washing steps unless specifically noted. After three washes, particles are resuspended in TE buffer with 1% PVA and lyophilized for long term storage.

### 4. Surface Hybridization and Step-by-Step Conjugation to Attach Biomolecules

Lyophilized particles are reconstituted in water, and measured the optical density at 550 nm as an indication of particle concentration. NH₂-modified DNA strands (Biosearch Technologies) complementary to the scaffolds are added at ~200 nM/OD₅₅₀ and incubated at 37° C. for 30 mins for hybridization, followed by centrifugations to wash off unhybridized DNA. A large excess of MAL-dPEG₄-NHS linker (Quanta Biodesign #10214) is added at 6 μM/OD₅₅₀ and incubated at RT for 1 hour to endow the particles with thiol-reactive function, followed by three washes to remove the excess. Biomolecules with free thiols are then added at 50 nM/OD₅₅₀ and incubated at RT for 1 hour to conjugate to the surface of particles. Particles with biomolecules loaded are washed for three times and lyophilized in PBS buffer with 1% PVA supplemented. Biomolecules labeled with fluorescent dyes, once loaded on particles, are analyzed the efficiency by dissolving the particles in 95% Dimethyl sulfoxide (DMSO) and further diluting for 10 folds in PBS for fluorescence-based quantification.

To fabricate control particles without DNA scaffold, 100 nmol block-co-polymer PLGA-PEG-MAL (Akina #AI053) is mixed with 50 mg PLGA (Sigma #719900) in the solvent composed of 0.5 mL ethyl acetate and 0.5 mL H₂O. Following the probe-sonication process described above, the particles formed are filtered through 40 μm filter (Sigma #CLS431750), and washed by centrifugation. MAL-presenting particles are then added with biomolecules with free thiol exposed at 50 nM/OD₅₅₀ and incubated at 37° C. for 1 hour for conjugation.

### 5. Attachment of DNA to Antibody or Fc-Tagged Protein and its Purification

Antibody (anti-PD-L1, Bio-X-Cell #BE0285; anti-CD28, Bio-X-Cell #BE0248) or Fc-tagged protein (HER2, Acro Biosystems #HE2-H5253) is exchanged the buffer to the reducing buffer (PBS with 10 mM EDTA supplemented) by size exclusion chromatography (Zeba Spin Desalting Column, Thermo Scientific #89882), and selectively reduced the disulfide bond at the hinge region by adding TCEP with 4.5 molar excess and incubating at 37° C. for 1 hour. Excess amount of TCEP is removed through size exclusion chromatography. 3′-NH₂ modified DNA complementary to the scaffold (Biosearch Technologies) is conjugated with MAL-dPEG₄-NHS linker (Quanta Biodesign #10214) with 30-fold molar excess in HEPES buffer (pH 7.0) at 37° C. for 1 hour, followed by the removal of the unconjugated linker through 70% ethanol precipitation and size exclusion chromatography. Reduced antibody or Fc-tagged protein, and conjugated DNA are combined with the molar ratio of 1:10, and incubated at 37° C. for 1 hour and 4° C. for overnight. The next day, DNA-protein conjugates are purified using protein G affinity chromatography (Genscript #L00209) to remove unconjugated DNA.

### 6. Attachment of DNA to His-Tag GFP and its Purification

His-tag GFP are expressed by Escherichia coli BL21 (DE3) (Novagen) transduced with pRSET-EmGFP vector (ThermoFisher, #V35320) in E. coli expression medium (MagicMedia, Invitrogen #K6803), and extracted using cell lysis reagent (Sigma, #B7435) followed by the purification using nickel-nitrilotriacetic acid affinity chromatography (Invitrogen #R90115). MAL-PEG₄-NHS linker is mixed with GFP at 30-fold molar excess, and incubated at 37° C. for 1 hour followed by the size-exclusion chromatography to remove the excess. Thiolated complementary DNA (Biosearch Technologies) is treated with TCEP at 100 molar excess at 37° C. for 1 hour to remove the protection cap and precipitated in 70% ethanol to remove excess amount of TCEP. Modified GFP and thiol-DNA are combined at 1:10 molar ratio and incubated at 37° C. for 1 hour and 4° C. for overnight. The next day, GFP-DNA conjugates are purified using nickel-nitrilotriacetic acid affinity chromatography to remove unconjugated DNA.

### 7. Surface Loading of Biotinylated Proteins

3′-biotinylated DNA complementary to the scaffolds (Biosearch Technologies) is hybridized to the particle surface using the protocol described above. After that, a large excess of streptavidin (Prozyme #SA10) is added at 1.1 mg/mL per OD₅₅₀, mixed immediately, and incubated at RT for 30 mins, followed by three washes. Biotinylated antibody, protein or peptide is added subsequently and incubated at RT for 30 mins to bind with streptavidin for particle surface loading followed by three washes.

8. Surface Functionalization of Polymeric Particles with Multiple Proteins at Intended Ratio

3′ Thiolated DNA with different sequences are synthesized: R (5′AGTGGGAGCGCGTGATG3′, SEQ ID NO:1); G (5′GTTCATCTGCACCACCG3′, SEQ ID NO: 2); B (5′GCCTTTACGATGTCCTT3′, SEQ ID NO:3). Following the conjugation with PLGA-PEG-MAL (Akina, #AI053), polymer-DNA with different sequences at intended ratios, together with mainstream polymer and solvents, are mixed and fabricated the particles as described above. Complementary DNA strands pre-conjugated with proteins (described above) or biotinylated complementary strand (Biosearch Technologies) are hybridized onto the particles with 60 nM/OD₅₅₀ total and the ratios same as the input ratio during fabrication. For the proportion of biotinylated DNA strand, streptavidin and biotinylated protein/peptide/antibody are assembled on the particles surface as described above. Surface protein species are quantified the loading through the fluorescently labeled DNA part or protein part.

### 9. Enzymatic Challenge Assay

Particles hybridized with fluorescently labeled complementary strands, with or without IgG coverage are treated with DNase (RQ1 RNase-free DNase, Promega #M6101) at 5 U per 1 OD₅₅₀×50 μL and incubated at 37° C. for 20 mins, followed by the centrifugation at 10,000×g for 10 mins to analyze the supernatant fluorescence signal.

### 10. IVIS Imaging-Based Particle Stability Assay

NH₂-modified PLGA polymer (Poly(lactide-co-glycolide)-NH₂, LG 50:50, Mw 30,000-40,000 Da) dissolved in DMF is reacted with IR800CW-NHS Ester (Li-COR #P/N 929-70020) dye with 30-fold molar excess at RT for 1 hour, followed by the repeated 70% ethanol precipitation and DMF re-dissolving to remove unconjugated dye. 1 mg of IR800CW-labeled polymer is mixed with 50 mg mainstream polymer and 100 nmol PLGA-DNA to fabricate particles with 1-5 μm diameter using the protocol described above. 5′Quasar705-modified complementary strand (Biosearch Technology) is hybridized to track the surface DNA scaffold, while IR800 for the core tracking. NSG mice (female, ~8-12-weeks old) are implanted with xenograft tumors—5×10⁶ K562 tumor cells subcutaneously on the left and right flank, respectively. 10 days after tumor implantation, 50 μL fluorescence-labeled particles at 200 OD₅₅₀ are injected intratumorally, and imaged under IVIS 100 preclinical imaging system (Xenogen #124262) every 3-4 hours for the first 48 hours and every 8 hours for the rest of the week. Images are analyzed using Living Image Software (PerkinElmer).

11. Encapsulation of Peptide and DNA in Particles with DNA-Scaffold

Polymeric PLGA particles with DNA scaffold on the surface as well as peptide/DNA in the core are fabricated using a double-emulsion method. 0.25 mg peptide (Genscript) and 50 nmol DNA (Bioresearch Technologies) dissolved in 50 μL PBS is combined with 50 mg PLGA (Sigma #719900) dissolved in 0.5 mL ethyl acetate, mixed and probe-sonicated at 7-8 W for 5×5 s with 10 s intervals on ice. Then 100 nmol amphiphilic polymer-DNA reconstituted in 100 μL solvent mixture (ethyl acetate:H₂O=1:1, v/v) is added along with 400 μL aqueous buffer (10 mM sodium citrate, 600 mM Na+, pH3.0). The whole mixture is quickly vortexed and probe-sonicated at 7-8 W for 5×5 s with 10 s intervals on ice, and immediately added with 9 mL 0.2% PVA and stirred in the hood for 3 hours for ethyl acetate to evaporate. Particles are filtered through 40 μm filter, and centrifuged at 10,000×g for 10 mins to collect the pellet, and then resuspended in TE buffer with 0.01% Tween-20 for washing. After three washes, particles are resuspended in TE buffer with 1% PVA and lyophilized for long term storage.

### 12. Primary Human T Cell Isolation and Culture

Primary CD4+ and CD8+ T cells are isolated from anonymous donor blood after apheresis by negative selection (STEMCELL Technologies #15062 and #15063). Blood is obtained from Blood Centers of the Pacific, as approved by the University Institutional Review Board. T cells are cryopreserved in RPMI-1640 (UCSF cell culture core) with 20% human AB serum (Valley Biomedical, #HP1022) and 10% DMSO. After thawing, T cells are cultured in human T cell medium consisting of X-VIVO 15 (Lonza #04-418Q), 5% Human AB serum, and 10 mM neutralized N-acetyl L-Cysteine (Sigma-Aldrich #A9165) supplemented with 30 units/mL IL-2 (NCI BRB Preclinical Repository) for all experiments.

### 13. Transduction of Synthetic Notch CAR T Cells

SynNotch receptor is built by fusing the LaG17 nanobody to the mouse Notch1 (NM_008714) minimal regulatory region (Ile1427 to Arg1752) and Gal4 DBD VP64. It also contains an N-terminal CD8a signal peptide (MALPVTALLLPLALLL HAARP, SEQ ID NO:4) for membrane targeting and a myc-tag (EQKLISEEDL, SEQ ID NO:5) for easy determination of surface expression with a-myc A647 (cell-signaling #2233). The receptors are cloned into a modified pHR'SIN:CSW vector containing a PGK promoter for all primary T cell experiments. The pHR'SIN:CSW vector is also modified to make the response element plasmids. Five copies of the Gal4 DNA binding domain target sequence (GGAGCACTGTCCTCC GAACG, SEQ ID NO: 6) are cloned 50 to a minimal CMV promoter. Also included in the response element plasmids is a PGK promoter that constitutively drives mCherry expression to easily identify transduced T cells. For inducible HER2-CAR vectors, the CARs are cloned via a BamHI site in the multiple cloning site 30 to the Gal4 response elements. All constructs are cloned via in fusion cloning (Clontech #ST0345).

Pantropic VSV-G pseudotyped lentivirus is produced via transfection of Lenti-X 293T cells (Clontech #11131D) with a pHR'SIN:CSW transgene expression vector and the viral packaging plasmids pCMVdR8.91 and pMD2.G using Fugene HD (Promega #E2312). Primary T cells are thawed the same day and, after 24 hours in culture, are stimulated with Human T-Activator CD3/CD28 Dynabeads (Life Technologies #11131D) at a 1:3 cell:bead ratio. At 48 hours, viral supernatant is harvested and the primary T cells are exposed to the virus for 24 hours. At day 4 after T cell stimulation, the Dynabeads are removed, and the T cells expanded until day 9 when they are rested and could be used in assays. T cells are sorted for assays with a Beckton Dickinson (BD) FACs ARIA II. AND-gate T cells exhibiting basal CAR expression were gated out during sorting.

### 14. Cancer Cell Lines

The cancer cell lines used are K562 myelogenous leukemia cells (ATCC #CCL-243) and A375 malignant melanoma cells (ATCC #CRL-1619). K562 and A375 are lentivirally transduced to stably express human HER2 and GFP. All cell lines were sorted for expression of the transgenes. K562 cells are cultured in Iscove's Modified Dulbecco's Medium with 10% fetal bovine serum, and A375 cells are cultured in Dulbecco's Modified Eagle's Medium with 10% fetal bovine serum.

### 15. T Cell In Vitro Stimulation and Proliferation Assay

For all in vitro synNotch T cell assay, 2.5×10⁴ T cells are co-cultured with target cancer cells at a 1:1 ratio, together with PLGA microparticles at 0.075 OD₅₅₀×200 μL final (composed of 100 μL T cell medium and 100 μL cancer cell medium). After mixing the T cells and cancer cells in round bottom 96-well tissue culture plates, the cells are centrifuged for 2 min at 300 g to force interaction of the cells. The cultures are analyzed at 24 hours for markers of activation (e.g., CD69) for T cells. For proliferation assay, T cells are pre-stained with CellTrace Violet Cell Proliferation Kit (Invitrogen #34557) before the co-culturing, and analyzed at 96 hours. All flow cytometry analysis was performed in FlowJo software (TreeStar).

### 16. Cytokine Analysis

Primary CD4+ or CD8+ synNotch AND-Gate T cells are stimulated with the target cancer cell line and PLGA microparticles as described above for 48 hours and supernatant is harvested. IL-2 levels in the supernatant of CD4+ T cells are determined via IL-2 ELISA (eBiosciences #BMS2221HS), and IFN gamma levels from CD8+ T cells are determined via IFN gamma ELISA (Invitrogen, #KHC4021).

### 17. In Vitro Target Cell Killing Assay

For all in vitro target cell killing assay, 2.5×10⁴ A375 cells are seeded on flat-bottom 96-well tissue culture plate and cultured for 8 hours to settle, then CD8+ T cells are co-cultured with cancer cells at 1:1 ratio, together with PLGA microparticles at 0.075 OD₅₅₀×200 μL final. 48 hours later, cells are gently washed with PBS for 2 times, and PrestoBlue Cell Viability Reagent (Invitrogen #A13262) is added to analyze the cell viability.

### 18. In Vivo Tumor Targeting

NSG mice are implanted with two xenograft tumors—5×10⁶ GFP+ K562 tumor cells subcutaneously on the left and right flank, respectively. Seven days after tumor implantation, 5×10⁶ primary human CD4+ and CD8+ T cells (1×10⁷ total T cells) are injected intravenously into the mice. These T cells were either untransduced (control) or engineered with the a-GFP synNotch Gal4VP64 receptor and the corresponding response elements regulating HER2 4-1BBδ CAR expression. Functionalized PLGA microparticles are injected intratumorally at one side of the two flanks, leaving the other as the control. Tumor size is monitored via caliper over 20 days after T cell injection. For Kaplan-Meier experiments, the same protocol is used but single tumors are injected into the mice. Mice are considered dead when the tumor size reaches euthanasia criteria.

### Example 1: Ex Vivo Enrichment and Expansion of Chimeric Antigen Receptor T Cells Using Antigen-Presenting Biomaterial

Chimeric antigen receptor (CAR) T cells have shown great promise in clinics, but their manufacturing still faces significant challenges in obtaining cell products with optimal quantity and quality for patient treatment. This invention describes a new strategy of single or repeated stimulation of human CAR-T cell ex vivo using functionalized biomaterials (CAR antigen-presenting particles, CAPP) that can selectively expand CAR-T cells over a long period of time (>100 days). The massively expanded cells ideally retain their cytotoxicity and effector function upon antigen-specific target cell exposure in vitro. In contrast to the typical stimulation by antigen-expressing cancer cell lines, the stimulation by biomaterials uncoupled cell proliferation from proinflammatory cytokine secretion and cell exhaustion/dysfunction. This may be induced by the unique physical and chemical signals provided during T cell activation, and various formulations of biomaterials (e.g., spherical particle size and composition, surface antigen density, and co-presentation of co-stimulatory signals) sharing key features can achieve the similar phenotypic outcome of T cells.

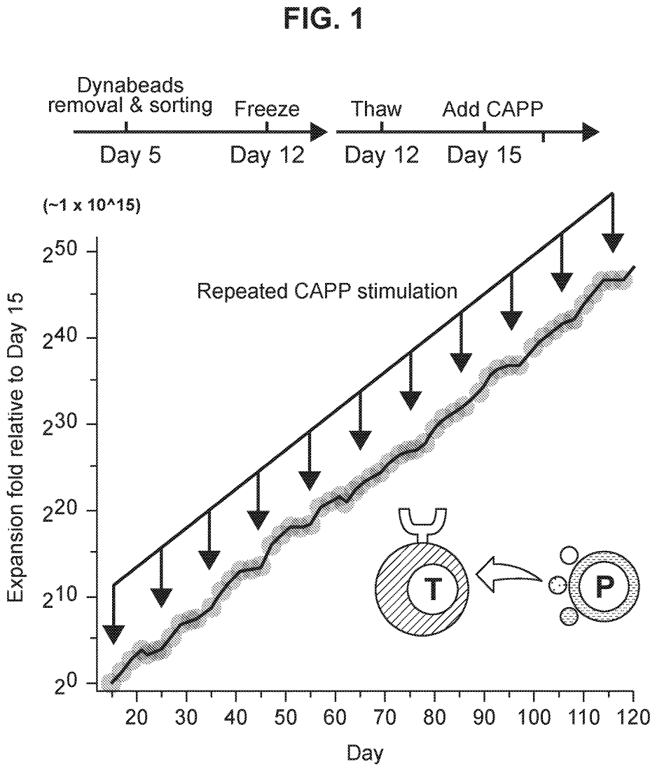

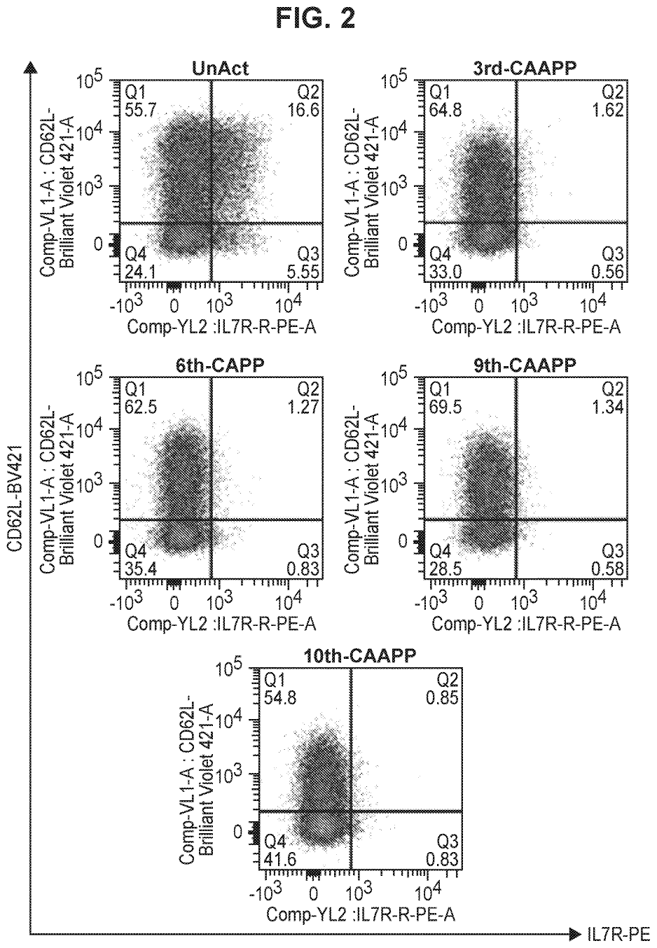

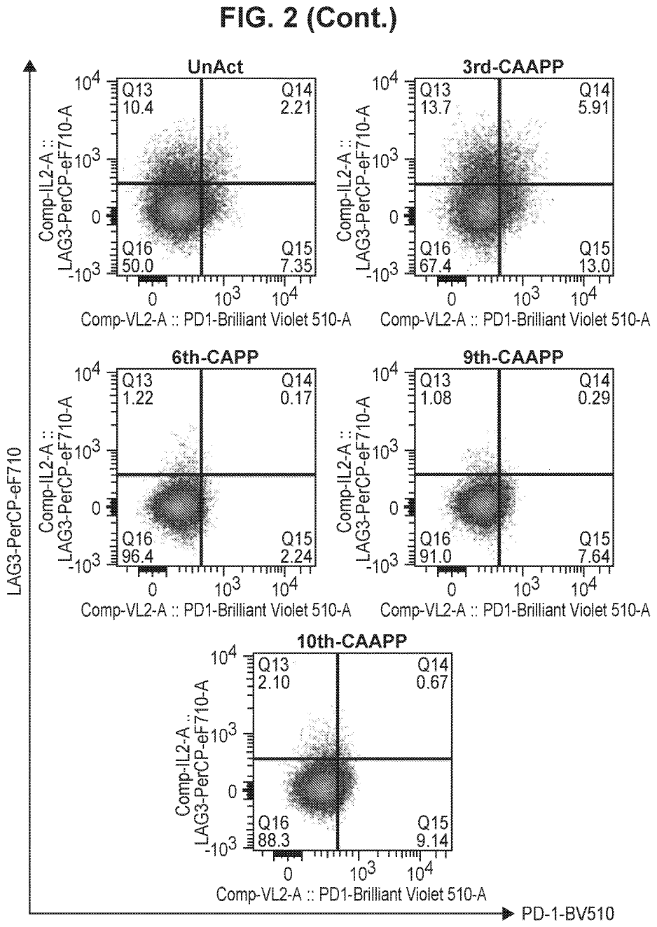

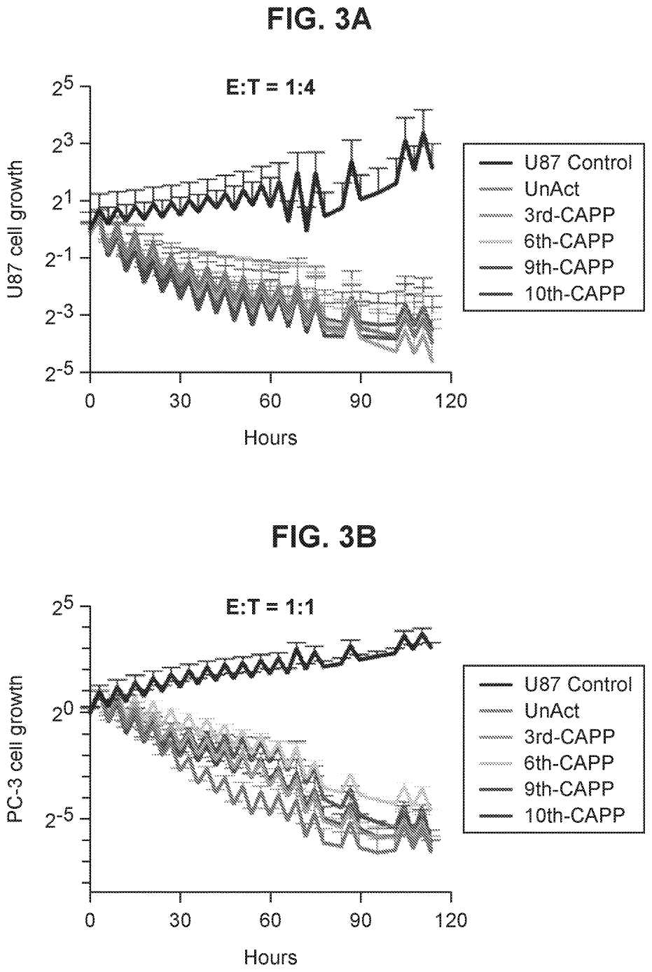

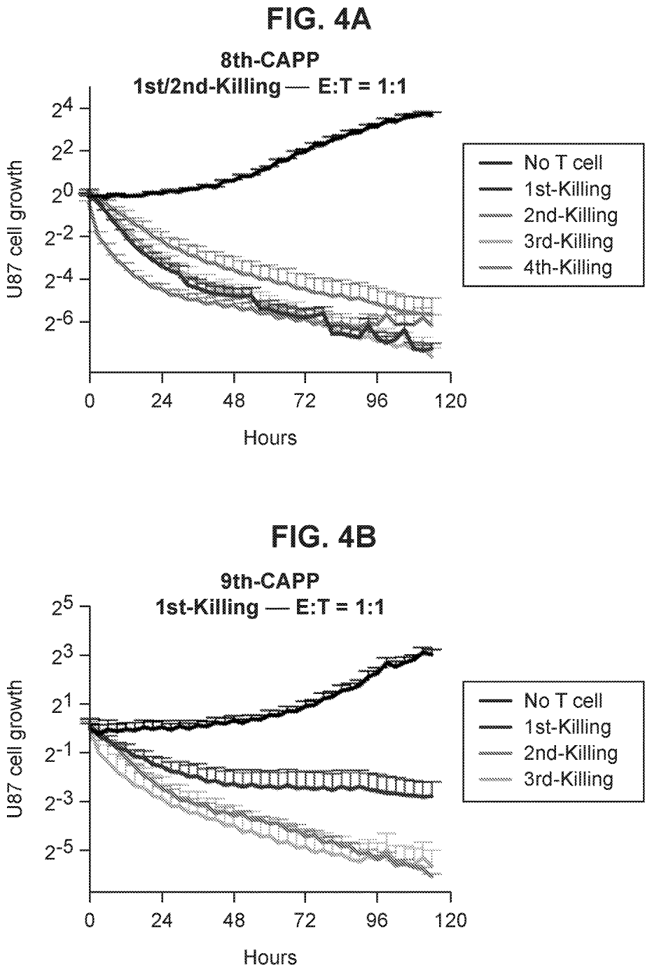

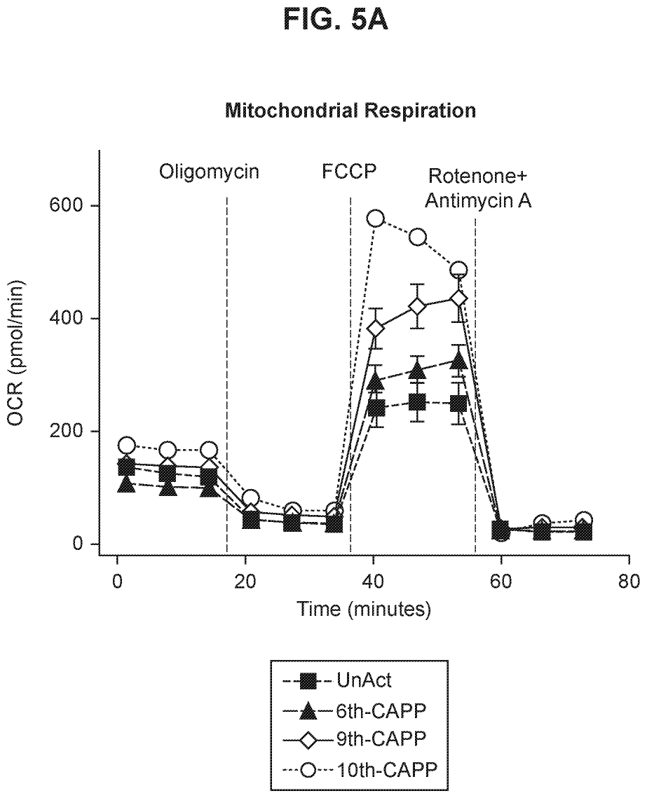

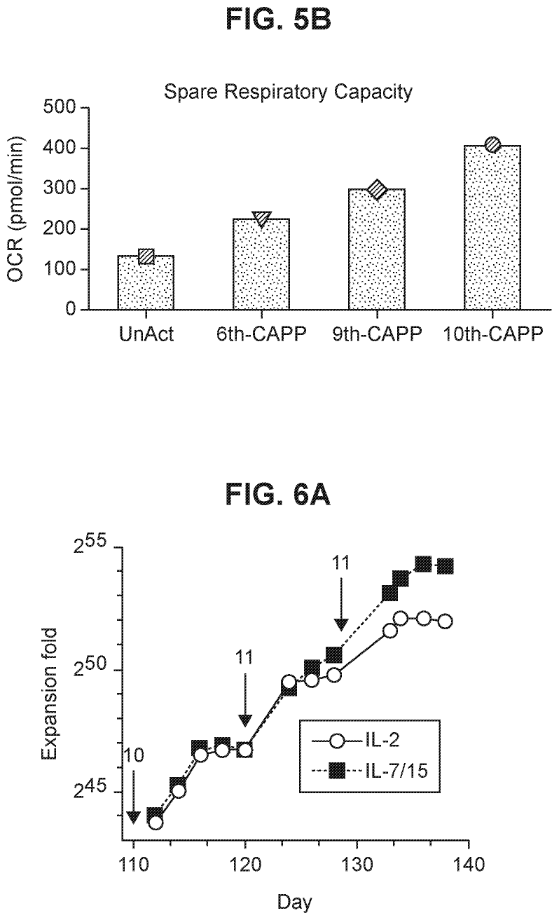

For example, a repeated stimulation (every 10 days for 10 rounds) of CAR-T cells with an anti-EGFR nanobody plus CD8 transmembrane, CD28 costimulatory, and CD3z domains manufactured from a healthy human donor led to a sustained cell proliferation with ~1×10¹⁵ fold expansion at day 110-120 (FIG. 1). The expression level of CD62L, an essential marker on memory T cells, was retained with multiple rounds of CAPP-stimulation, while the levels of PD-1 and LAG-3, typical exhaustion markers, stayed low (FIG. 2). The yielded cells from repeated rounds of CAPP stimulation (3, 6, 9, and 10) showed a similar level of cytotoxicity against target cell lines including U87 (glioblastoma) and PC-3 (prostate cancer) (FIG. 3). Also, they are effective in multiple rounds of target killing (FIG. 4). Meanwhile, the seahorse metabolic activity analysis showed that cells gained elevating levels of spare respiratory capacity with more rounds of CAPP stimulation, suggesting an ideal fitness of the expanded cells (FIG. 5).

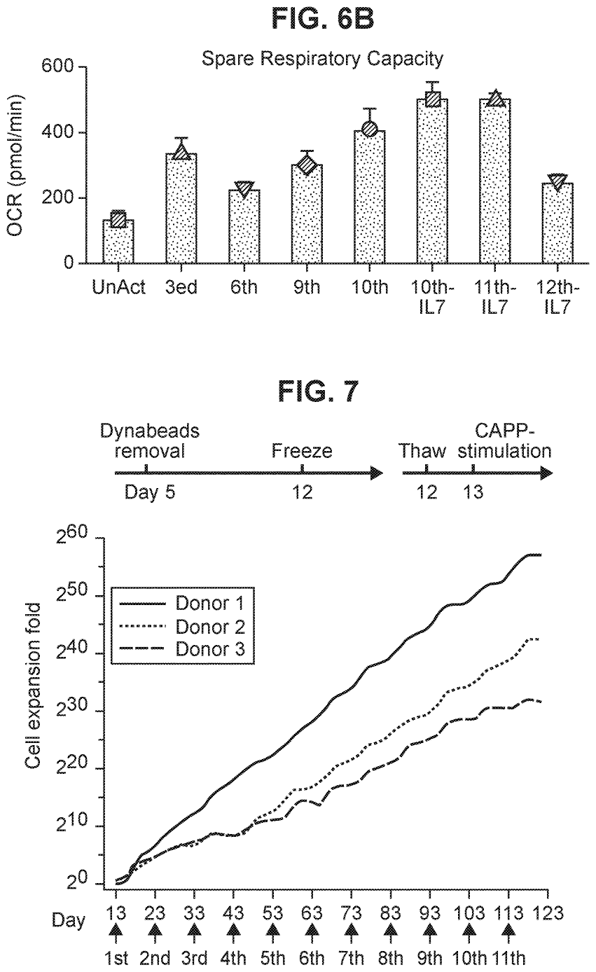

The cytokine IL-2 was provided at 30 U/mL throughout the CAPP stimulation to sustain the cell proliferation, and it can be replaced by providing a combination of IL-7 and IL-15 cytokines (FIG. 6A). Indeed, IL-7 and IL-15 sustained for longer cell proliferation after 110 days (FIG. 6A), and the spare respiratory capacity is higher for cells grown in IL-7/IL-15 than in IL-2 (FIG. 6B). Donor-to-donor variance is another major issue affecting T cell manufacturing, and our data show that this strategy can potentially overcome the challenge to achieve sustainable proliferation property among different donors (FIG. 7).

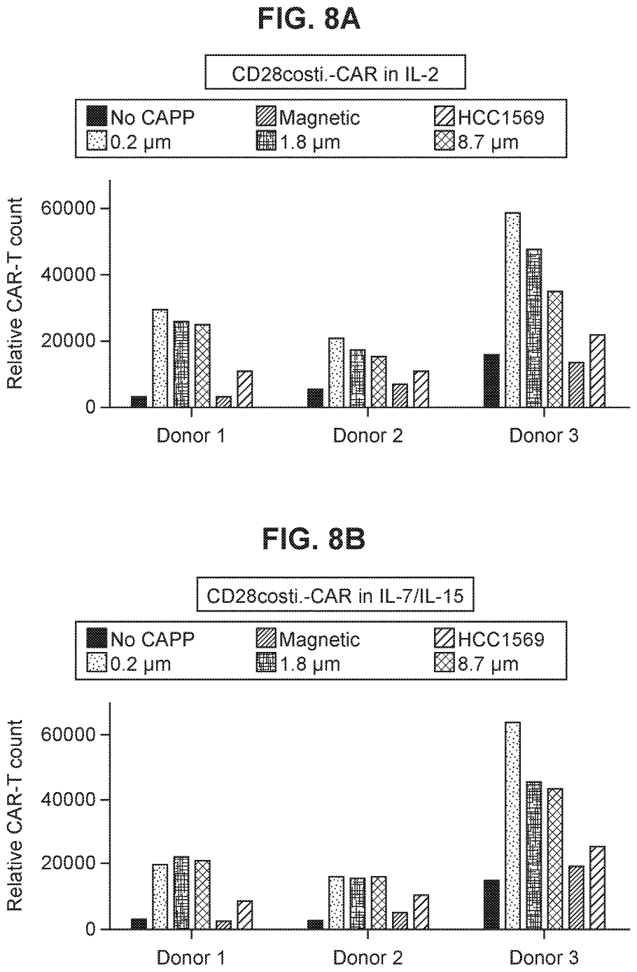

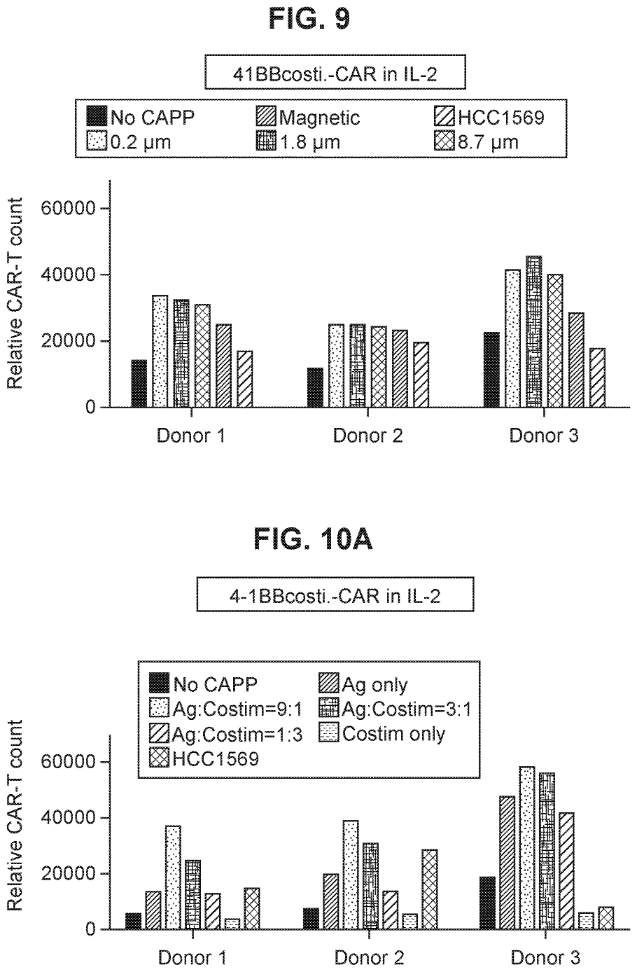

The CAPP-stimulated CAR-T cell proliferation has been reproduced in various CAR constructs covering different antigens (e.g., EGFR, CD19, CD22, HER2) and with different co-stimulatory domains (4-1BB and CD28, the two popular constructs in clinical uses). However, the features of CAPP materials including size and dimension (e.g., from nanometer to micron to scaffold matrix), composition (e.g., polymer, metal, lipids), antigen density, and co-presentation of costimulatory ligands at specific ratios to activate co-stimulatory receptors (e.g., CD28, 4-1BB, ICOS, etc.), would need to be optimized for specific CAR constructs to achieve the sustained proliferation. For example, antigen-coated magnetic particles did not induce cell proliferation as well as CAPP made of PLGA polymers for CAR-T cells with CD28 costimulatory domain (FIG. 8), however, they did induce decent cell proliferation for CAR-T cells with the 4-1BB costimulatory domain (FIG. 9).

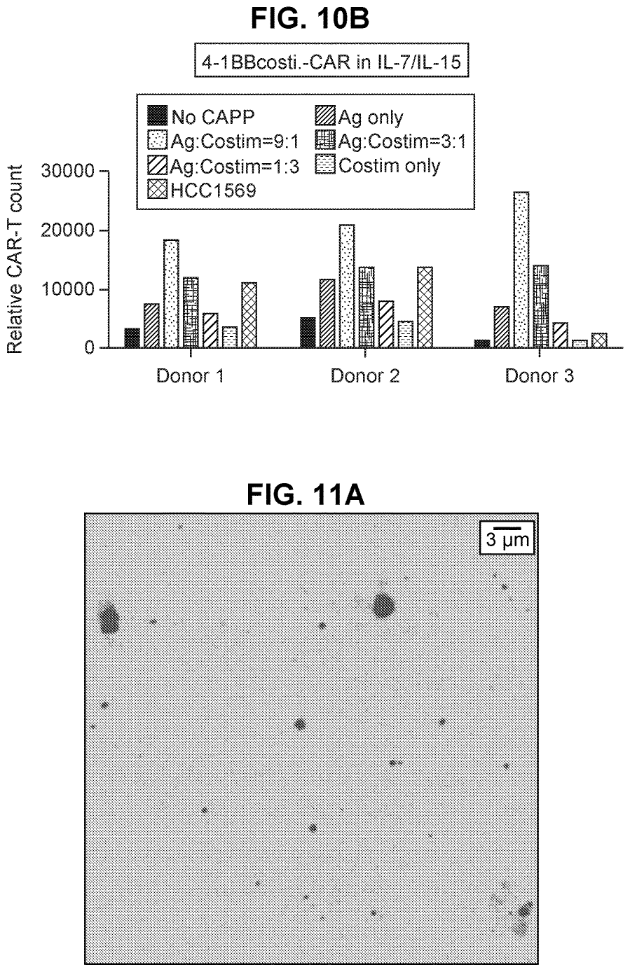

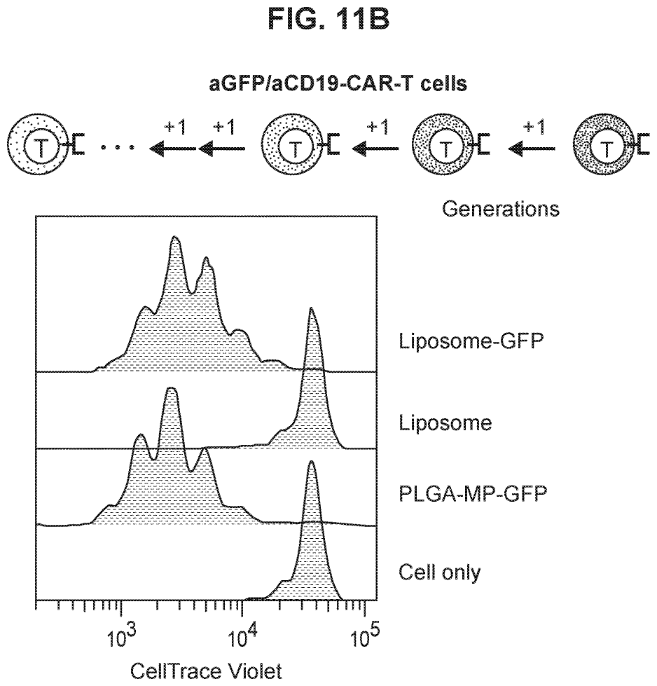

For the 2^{nd} generation CAR construct with the 4-1BB costimulatory domain, we found that the co-presentation of antigen with anti-CD28 antibody (CD28 agonist antibody) at a 9:1 ratio on CAPP surfaces significantly improves the T cell expansion among three T cell donors (FIG. 10). Antigen coated on liposomes can also achieve similar CAR-T cell expansion through antigen-specific stimulation (FIG. 11), and this approach can also be used for repeated stimulations to achieve sustained proliferation.

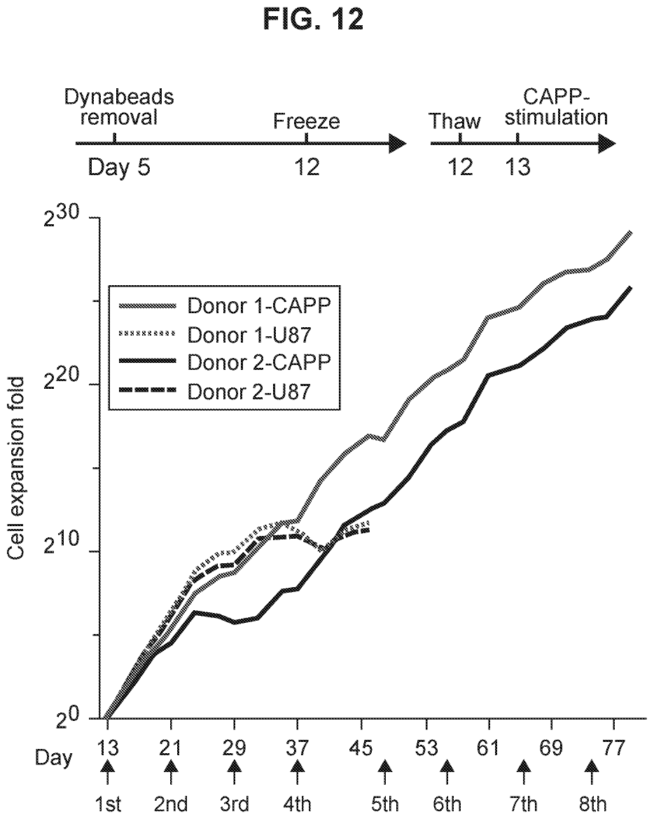

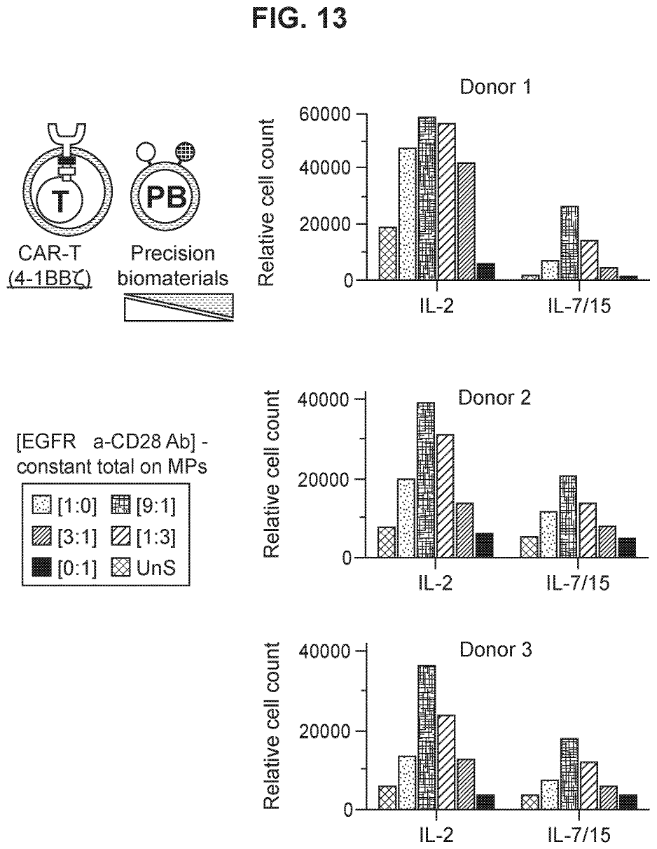

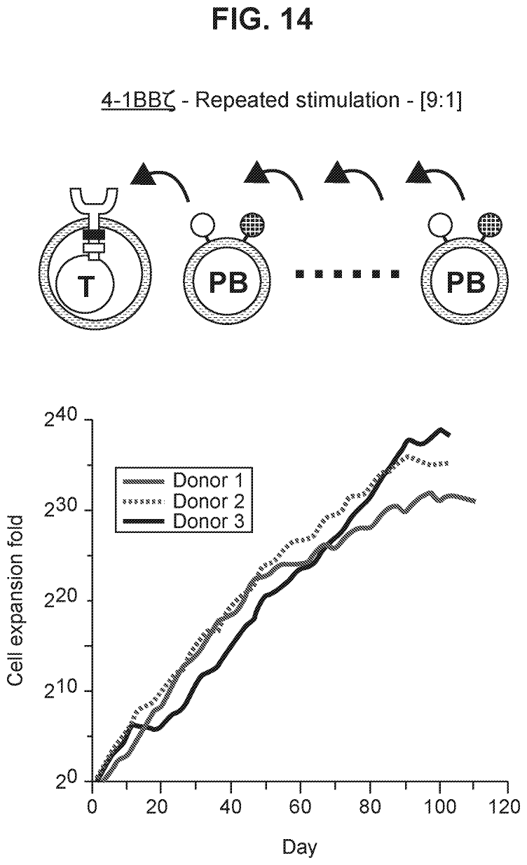

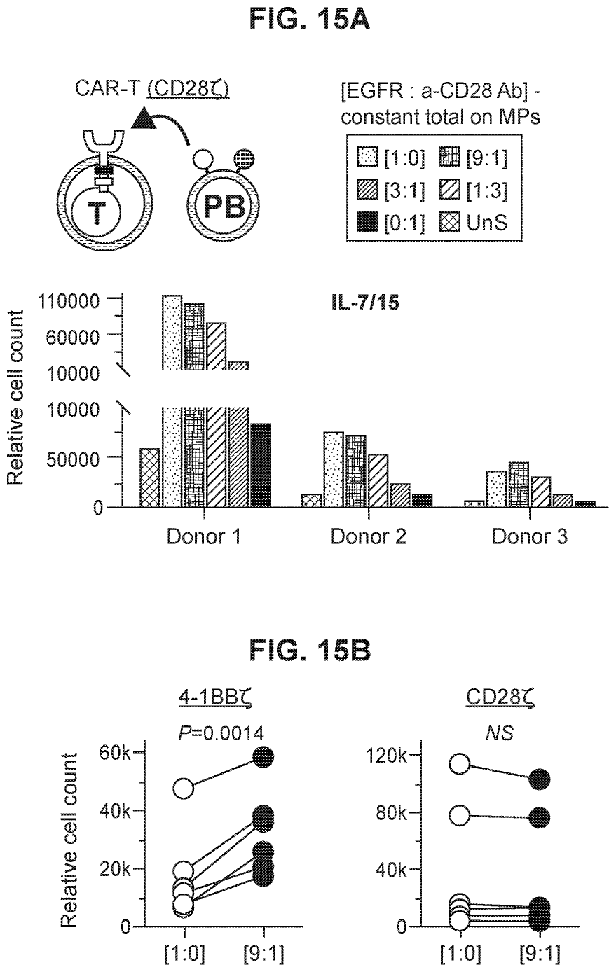

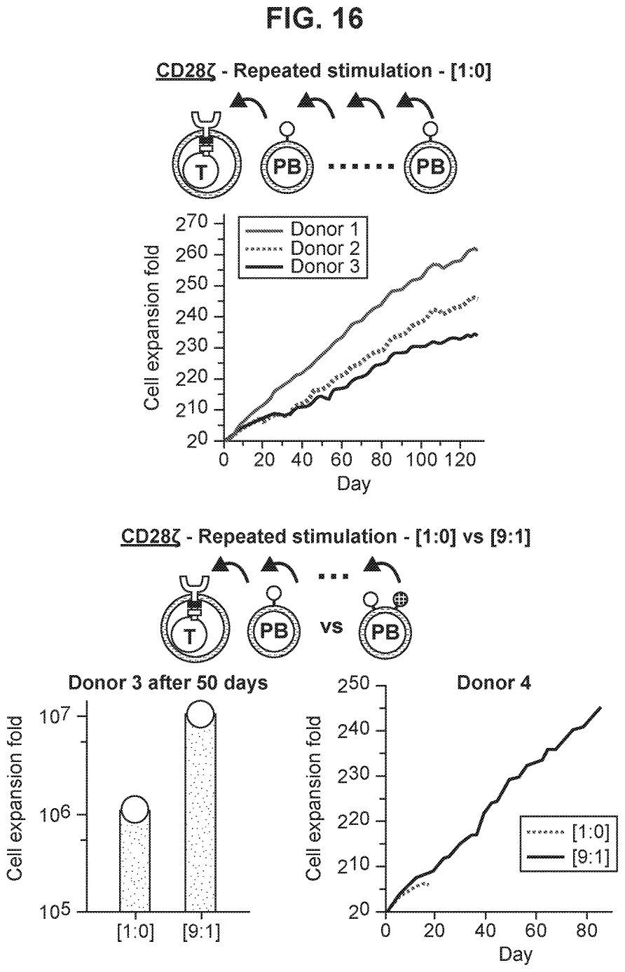

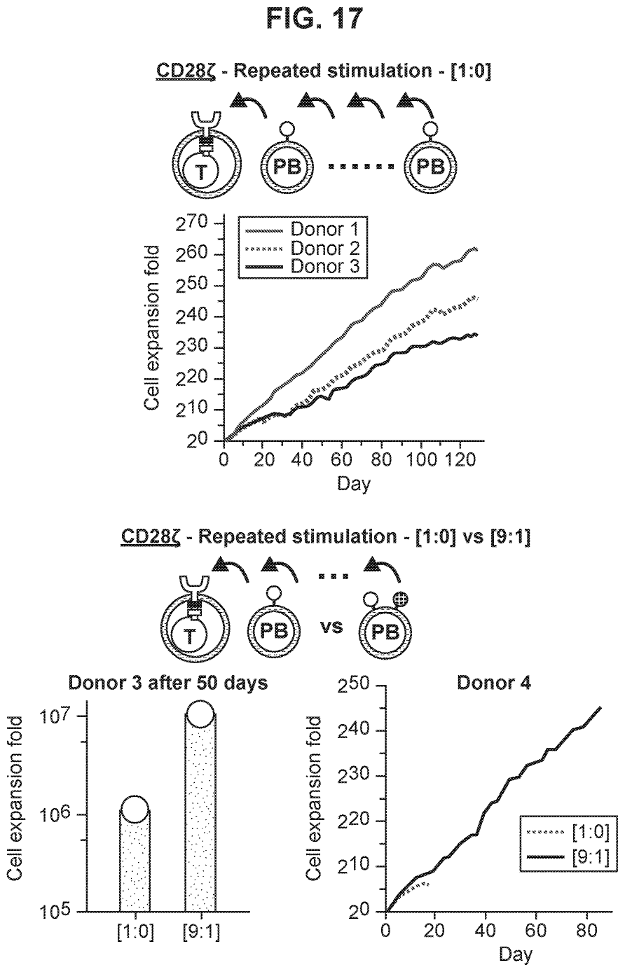

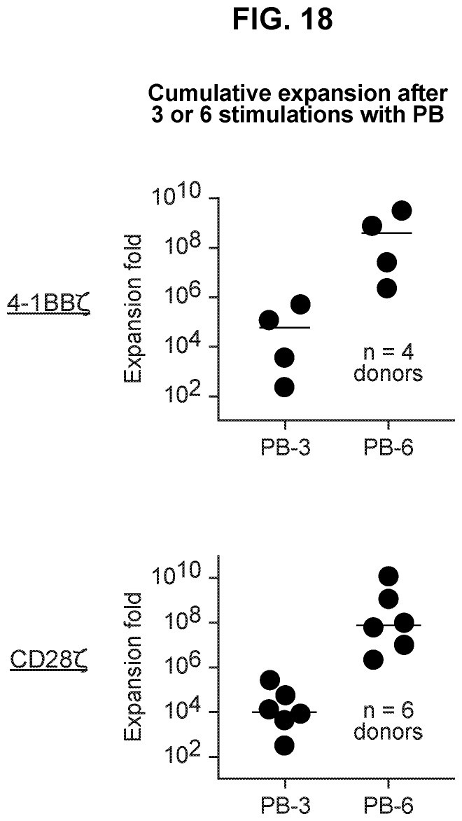

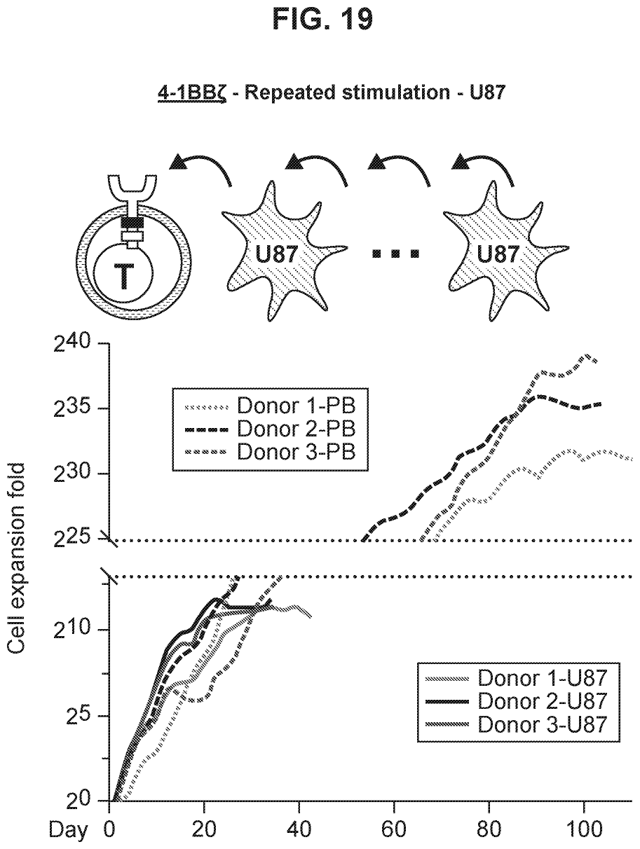

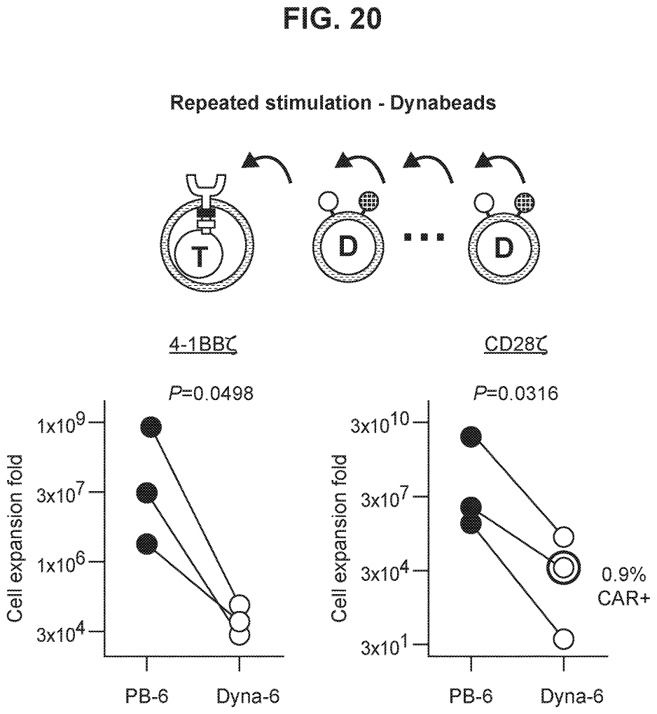

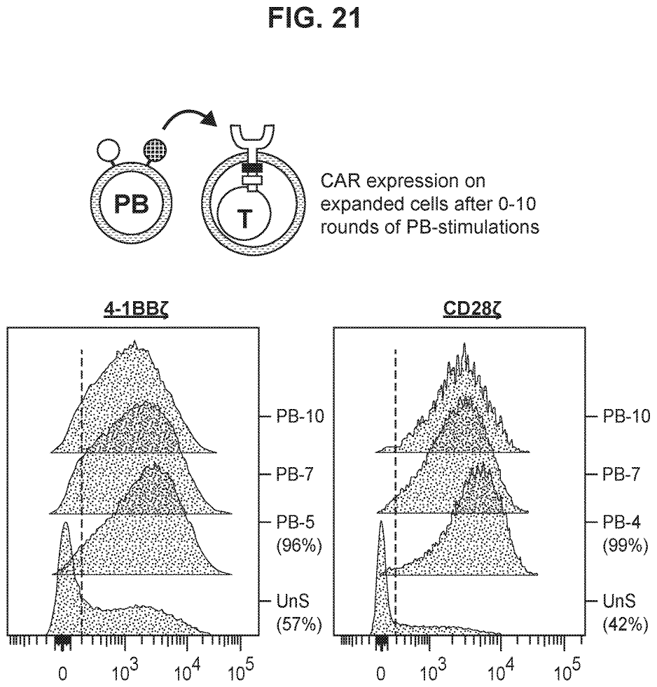

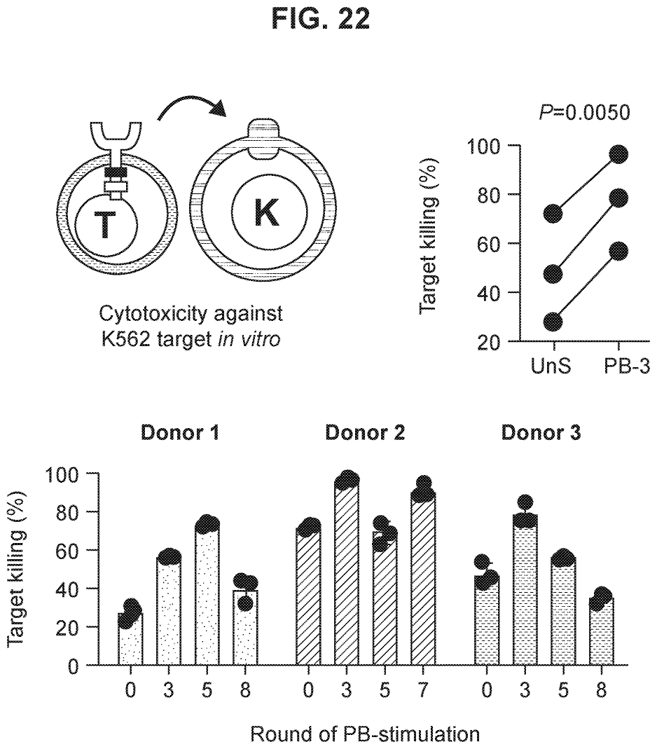

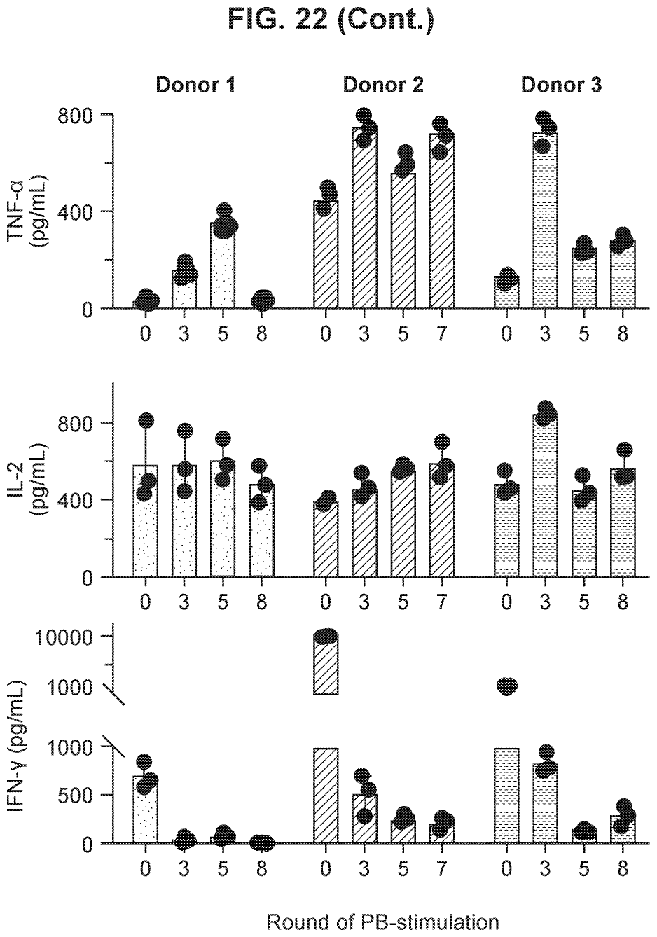

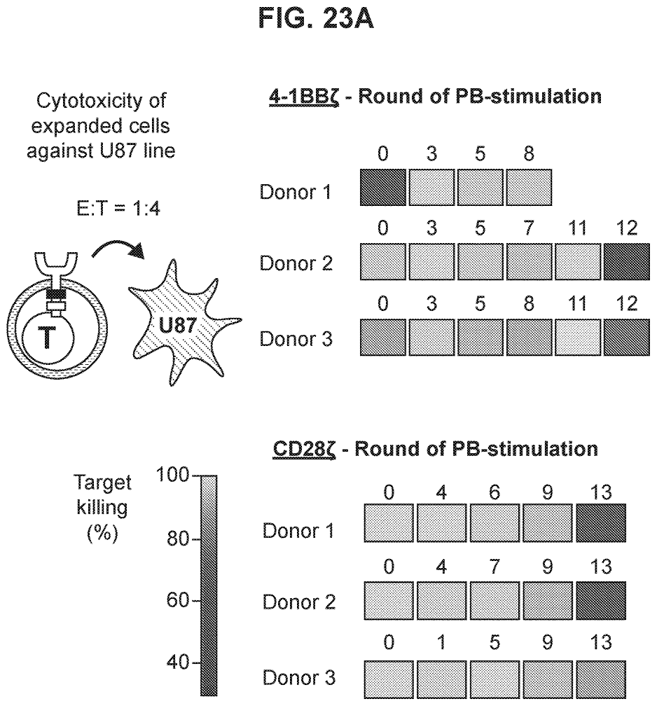

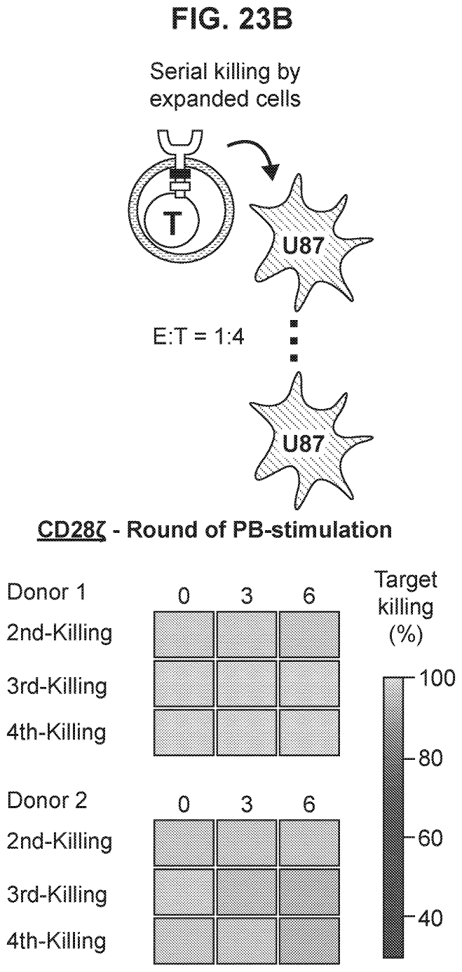

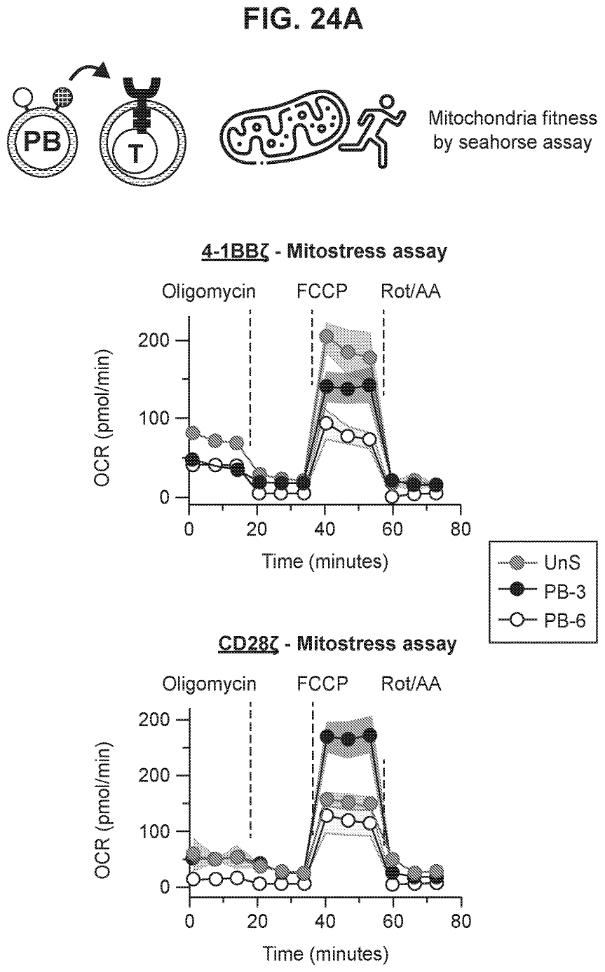

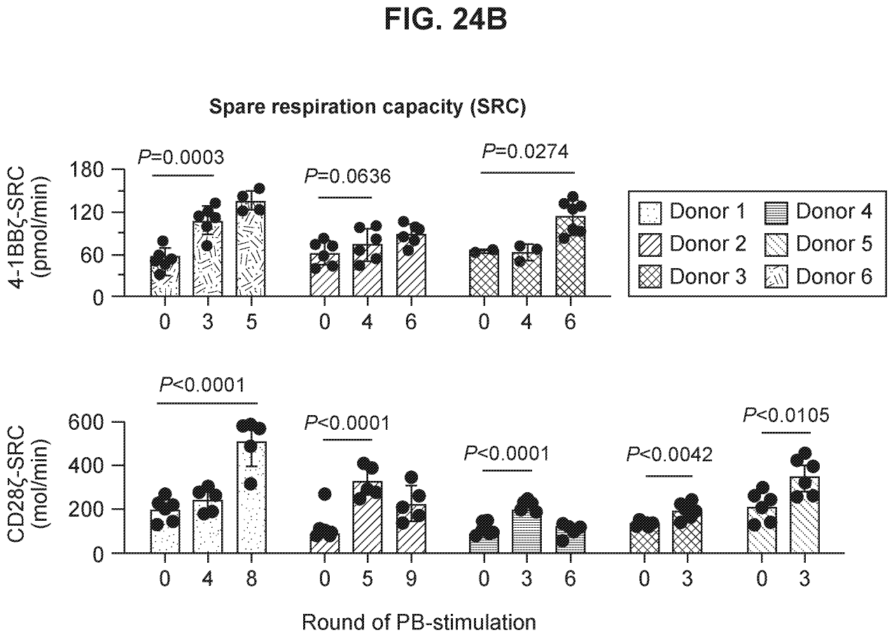

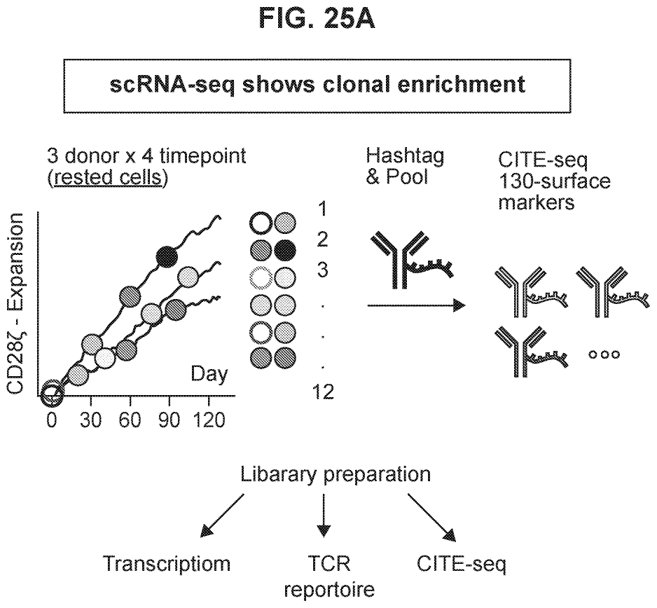

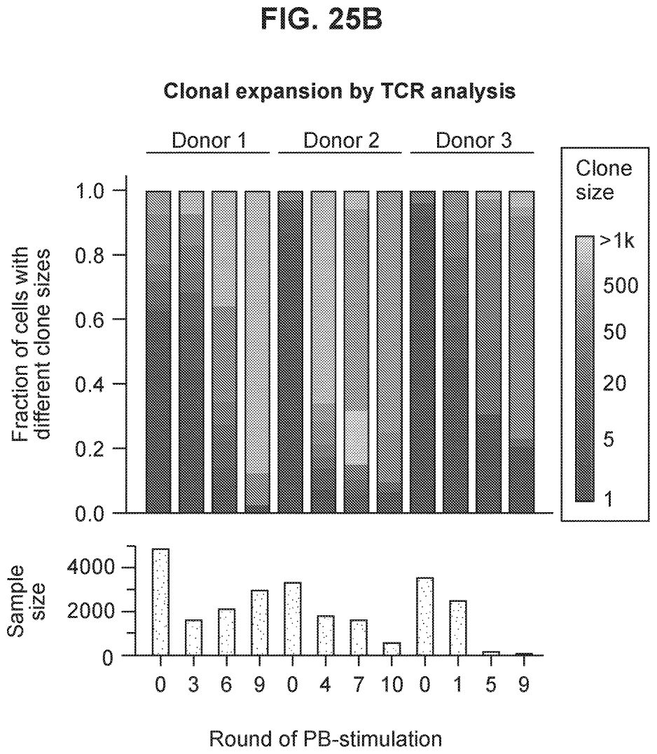

For the 2^{nd} generation 4-1BB costimulatory CAR, CAPP with the surface co-loaded antigen and anti-CD28 antibody at 9:1 ratio show the capability of maintaining cell proliferation with repeated stimulations, while antigen-only CAPP particles failed to achieve this (FIG. 12).

The disclosed methods can be used to produce CAR-related cell products that are normally challenging to obtain in sufficient quantity for clinical treatment. For example, state-of-the-art cell engineering approaches to enhance safety, persistence, and efficacy have involved more complex genetic engineering, such as use of synthetic notch receptor and responsive element transduction, DNMT3a knockout, and c-Jun overexpression, which can cause severe limitation in the cell quantity produced. For autologous CAR-T cells, the disclosed methods can be used to enrich cell clones that have better sustainability. In addition, the methods, described herein, can be used for “off-the-shelf” universal allogeneic CAR-T cell engineering to enrich and proliferate cells from TCR knockout and other genomic engineering.

### Example 2: CAR-T Cell Proliferation Assay Protocol

Constitutive CAR-T cells are stained with CellTrace Violet dye (Cat #C34557) following instructions. CAR-antigen presenting particles, including PLGA nano-/micro-particles, liposomes or magnetic particles are combined with cells at 10:1 (particle to cell) ratio, and incubated at 37° C. for 4 days. Cells are then analyzed using flow cytometry for 5 proliferation.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of culture expansion of a chimeric antigen receptor (CAR)-T cell, the method comprising:
(a) culturing the CAR-T cell under suitable conditions for growth of the CAR-T cell;
(b) contacting the CAR-T cell with a synthetic CAR-antigen presenting particle to produce an antigen-stimulated CAR-T cell, wherein the synthetic CAR-antigen presenting particle comprises:
i) a polymeric core;
ii) a nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core; and
iii) a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to the CAR expressed on the CAR-T cell, wherein binding of the antigen to the CAR induces proliferation of the CAR-T cell;
(c) culturing the antigen-stimulated CAR-T cell in presence of interleukin-2 (IL-2), a combination of interleukin-7 (IL-7) and interleukin-15 (IL-15), or a combination of IL-2, IL-7, and IL-15, wherein the antigen-stimulated CAR-T cell proliferates; and
(d) repeating steps (b) and (c).

2.-4. (canceled)

5. The method of claim 1, wherein
a) the polymeric core comprises poly(D,L-lactide-co-glycolide) (PLGA), poly(lactic acid) (PLA), polyglycolic acid (PGA), poly(e-caprolactone) (PCL), or polyethylene glycol (PEG), or a combination thereof;
b) the polymer of the nucleic acid-polymer conjugate comprises a poly(D,L-lactide-co-glycolide) (PLGA)-polyethylene glycol (PEG) block polymer (PLGA-block-PEG) or a poly(D,L-lactide) (PLA)-polyethylene glycol (PEG) block polymer (PLA-block-PEG) or a poly(e-caprolactone) (PCL)-polyethylene glycol (PEG) block polymer (PCL-block-PEG); or
c) both a) and b).

6. (canceled)

7. The method of claim 1, wherein
a) the first single stranded nucleic acid comprises deoxyribonucleic acid (DNA) or ribonucleic acid (RNA) or peptide nucleic acid (PNA) and comprises 5-200 bases;
b) the second single stranded nucleic acid comprises deoxyribonucleic acid (DNA) or ribonucleic acid (RNA) or peptide nucleic acid (PNA), optionally wherein the DNA or RNA or PNA comprises 5-200 bases; or
c) both a) and b).

8.-9. (canceled)

10. The method of claim 1 any one of claims 1-9, wherein the first single stranded nucleic acid comprises at least 4 contiguous bases complementary to at least 4 contiguous bases in the second single stranded nucleic acid.

11. The method of claim 1, wherein the particle is a nanoparticle having a diameter ranging from 50 nm-500 nm, or from 0.5 μm-50 μm.

12. (canceled)

13. The method of claim 1, wherein the CAR comprises a transmembrane domain linked to an extracellular antigen binding domain and an intracellular signaling domain, wherein the extracellular antigen-binding domain specifically binds to the antigen.

14. The method of claim 13, wherein
a) the extracellular antigen binding domain comprises a single chain variable fragment (scFv), an antigen-binding fragment (Fab), a nanobody, a heavy chain variable (VH) domain, a light chain variable (VL) domain, a single domain antibody (sdAb), a shark variable domain of a new antigen receptor (VNAR), a single variable domain on a heavy chain (VHH), a bispecific antibody, a diabody, or a functional fragment thereof that binds specifically to the antigen;
b) the intracellular signaling domain is a CD3-zeta intracellular signaling domain or a ZAP-70 intracellular signaling domain; or
c) the intracellular signaling domain comprises an immunoreceptor tyrosine-based activation motif (ITAM);
d) the transmembrane domain is a CD8, CD28, Megf10, FcRy, Bail, MerTK, TIM4, Stabilin-1, Stabilin-2, RAGE, CD300f, integrin subunit av, integrin subunit ps, CD36, LRPl, SCARFI, CIQa, Axl, CD45, or CD86 transmembrane domain.

15.-16. (canceled)

17. The method of claim 1, wherein the CAR further comprises a costimulatory domain selected from 4-1BB, CD28, ICOS, OX-40, BTLA, CD27, CD30, GITR, or HVEM costimulatory domain.

18.-19. (canceled)

20. The method of claim 1, wherein the antigen is selected from the group consisting of: CD19, HER2, epidermal growth factor receptor (EGFR), green fluorescent protein (GFP), fluorescein isothiocyanate (FITC), CD19, CD20, CD22, CD38, CD30, CA125, MUC-1, prostate-specific membrane antigen (PSMA), CD44 surface adhesion molecule, mesothelin, carcinoembryonic antigen (CEA), EGFRvIII, vascular endothelial growth factor receptor-2 (VEGFR2), high molecular weight-melanoma associated antigen (HMW-MAA), MAGE-AI, IL-13R-a2, GD2, MET, GPC3, CD70, EphA2, EpCAM, CLDN18, BCMA, CA9, and fibroblast activation protein (PAP).

21. (canceled)

22. The method of claim 1, wherein the CAR-antigen presenting particle further comprises a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

23. The method of claim 22, wherein the costimulatory ligand is an anti-CD28 antibody, CD80, CD86, an anti-41BB antibody, an anti-ICOS antibody, or an anti-OX40 antibody wherein the ratio of the antigen to the costimulatory ligand on the surface of the CAR-antigen presenting particle is 1:20 to 20:1.

24. (canceled)

25. The method of claim 1, wherein the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle.

26. (canceled)

27. The method of claim 25, wherein the antigen and the cytokine are present at a ratio of 10:1 to 1:10.

28. The method of claim 1, further comprising contacting the CAR-T cell with a synthetic cytokine-presenting particle, wherein the synthetic cytokine-presenting particle comprises a cytokine presented on a surface of a synthetic particle.

29.-44. (canceled)

45. The method of claim 1, wherein expression of the CAR in the CAR-T cell is inducible.

46.-48. (canceled)

49. The method of claim 1, further comprising enriching for memory progenitor CAR-T cells generated from the antigen-stimulated CAR-T cell.

50. The method of claim 1, wherein step (b) is performed with a ratio of the synthetic CAR-antigen presenting particle to the CAR-T cell in a range of 2:1 to 10:1 or at least 5:1 or 10:1.

51.-102. (canceled)

103. A composition comprising a synthetic CAR-antigen presenting particle (CAPP), the CAPP comprising:
a. a polymeric core;
b. a first nucleic acid-polymer conjugate comprising a first single stranded nucleic acid covalently attached to a polymer, wherein the polymer is non-covalently associated with the polymeric core thereby presenting the first single stranded nucleic acid on a surface of the polymeric core;
c. a nucleic acid-antigen conjugate comprising a second single stranded nucleic acid covalently attached to an antigen, wherein the second single stranded nucleic acid is complementary to the first single stranded nucleic acid and is associated with the first single stranded nucleic acid via hybridization thereby presenting the antigen on the surface of the particle, wherein the antigen specifically binds to a CAR expressed on a CAR-T cell, wherein binding of the antigen to the CAR activates the CAR-T cell; and
d. a second nucleic acid-polymer conjugate comprising a third single stranded nucleic acid covalently attached to the polymer, wherein the polymer is non-covalently associated with the polymeric core thereby presenting the third single stranded nucleic acid on the surface of the polymeric core and a costimulatory ligand-nucleic acid conjugate comprising a fourth single stranded nucleic acid covalently attached to a costimulatory ligand, wherein the fourth single stranded nucleic acid is complementary to the third single stranded nucleic acid and is associated with the third single stranded nucleic acid via hybridization thereby presenting the costimulatory ligand on the surface of the polymeric particle.

104. (canceled)

105. The composition of claim 103, wherein the ratio of the antigen to the costimulatory ligand on the surface of the CAR-antigen presenting particle is 1:20 to 20:1.

106. The composition of claim 103, wherein the CAR-antigen presenting particle further comprises a third nucleic acid-polymer conjugate comprising a fifth single stranded nucleic acid covalently attached to the polymer, wherein the polymer is non-covalently associated with the polymeric core thereby presenting the fifth single stranded nucleic acid on the surface of the polymeric core and a cytokine-nucleic acid conjugate comprising a sixth single stranded nucleic acid covalently attached to the cytokine, wherein the sixth single stranded nucleic acid is complementary to the fifth single stranded nucleic acid and is associated with the fifth single stranded nucleic acid via hybridization thereby presenting the cytokine on the surface of the polymeric particle,
wherein the antigen and the cytokine are present at a ratio of 10:1 to 1:10.

107.-157. (canceled)
